(19) United States
(12) Patent Application Publication
(10) Pub. No.: US 2024/0254474 A1
(43) Pub. Date: Aug. 1, 2024

(54) METHODS, SYSTEMS, AND KITS FOR DUAL INDEXING NUCLEIC ACIDS

(71) Applicant: 10x Genomics, Inc., Pleasanton, CA (US)

(72) Inventor: Corey M. NEMEC, Fremont, CA (US)

(21) Appl. No.: 18/628,080

(22) Filed: Apr. 5, 2024

Related U.S. Application Data
(63) Continuation of application No. 17/573,350, filed on Jan. 11, 2022.
(60) Provisional application No. 63/136,546, filed on Jan. 12, 2021.

(51) Int. Cl. C12N 15/10; C12Q 1/6806; C12Q 1/6844; C12Q 1/6869

(52) U.S. Cl. CPC C12N 15/1065; C12Q 1/6806; C12Q 1/6844; C12Q 1/6869

(57) ABSTRACT

While robust multiplexed high-throughput systems allow for widespread use of genome-wide chromatin accessibility (e.g., using ATAC-seq) and other next-generation sequencing studies at single cell resolution, misassignment of index molecules between samples hampers the accuracy of the sequencing result. The present disclosure provides kits, methods, and compositions for dual-indexing to allow for accurate assignment of sequencing reads to their original source samples.

### CROSS REFERENCE

This application is a continuation of U.S. application Ser. No. 17/573,350, filed Jan. 11, 2022, which claims the benefit of U.S. Provisional Application No. 63/136,546, filed Jan. 12, 2021, each of which is incorporated herein by reference in its entirety.

### BACKGROUND

A sample may be processed for various purposes, such as identification of a type of moiety within the sample. The sample may be a biological sample. Biological samples may be processed, such as for detection of a disease (e.g., cancer) or identification of a particular species. There are various approaches for processing samples, such as polymerase chain reaction (PCR) and sequencing.

Biological samples may be processed within various reaction environments, such as partitions. Partitions may be wells or droplets. Droplets or wells may be employed to process biological samples in a manner that enables the biological samples to be partitioned and processed separately. For example, such droplets may be fluidically isolated from other droplets, enabling accurate control of respective environments in the droplets.

Biological samples in partitions may be subjected to various processes, such as chemical processes or physical processes. Samples in partitions may be subjected to heating or cooling, or chemical reactions, such as to yield species that may be qualitatively or quantitatively processed.

Although multiplexing allows widespread use of robust high-throughput systems for assaying chromatin accessibility (e.g., using ATAC-seq) and other next-generation sequencing applications at single cell resolution, misassignment of index molecules between samples hampers the accuracy of the sequencing result.

### SUMMARY

Recognized herein is a need for systems, methods, compositions, and kits that address at least the abovementioned problems. The systems, methods, compositions, and kits of the present disclosure may permit dual indexing and barcoding of single cell libraries (e.g., single cell ATAC libraries).

Disclosed herein, in some embodiments, are kits. In an aspect, a kit comprises: a nucleic acid barcode molecule comprising, from 5′ to 3′: (i) a first primer binding sequence and (ii) a barcode sequence, and (iii) a first tag binding sequence; a first primer molecule comprising, from 5′ to 3′: (i) a first sample identification sequence and (ii) a first primer sequence corresponding to the first primer binding sequence; and a second primer molecule comprising a second sample identification sequence different from the first sample identification sequence.

In some embodiments, the nucleic acid barcode molecule is coupled to a support. In some embodiments, the nucleic acid barcode molecule is coupled to the support at a 5′ end of the nucleic acid barcode molecule. In some embodiments, the support is a bead. In some embodiments, the support is a gel bead. In some embodiments, the support is coupled to a plurality of nucleic acid barcode molecules.

In some embodiments, the plurality of nucleic acid barcode molecules comprises the barcode sequence. In some embodiments, a nucleic acid barcode molecule of the plurality of nucleic acid barcode molecules comprises a unique molecular identification sequence that is different from that of another nucleic acid barcode molecule of the plurality of nucleic acid barcode molecules. In some embodiments, the nucleic acid barcode molecule is releasably coupled to the support via a labile moiety. In some embodiments, the labile moiety is cleavable via one or more stimuli selected from the group consisting of a thermal, chemical, and photo stimulus. In some embodiments, the labile moiety comprises a disulfide bond. In some embodiments, the first primer sequence of the first primer molecule does not hybridize to the first tag sequence. In some embodiments, the first primer binding sequence of the nucleic acid barcode molecule does not comprise the first tag binding sequence of the nucleic acid molecule. In some embodiments, the first primer binding sequence of the nucleic acid barcode molecule does not correspond to an R1 or R2 sequence. In some embodiments, the first primer binding sequence of the nucleic acid barcode molecule does not correspond to a flow cell adapter sequence. In some embodiments, the first tag binding sequence is complementary to a first tag sequence. In some embodiments, the kit further comprises a first additional nucleic acid barcode molecule comprising the first tag sequence, or complement thereof. In some embodiments, the kit further comprises a second additional nucleic acid barcode molecule comprising a second tag sequence different from the first tag sequence, or complement thereof.

In some embodiments, the primer molecule comprises a first adapter sequence. In some embodiments, the first adapter sequence is at a 5′ end of the first primer molecule. In some embodiments, the first adapter sequence is configured to attach to a flow cell of a sequencer. In some embodiments, the second primer molecule comprises, from 3′ to 5′: (i) a second tag binding sequence and (ii) the second sample identification sequence, and (iii) a second adapter sequence. In some embodiments, the second primer molecule comprises, from 3′ to 5′: (i) the second tag binding sequence and (ii) the second sample identification sequence and (iii) a second adapter sequence. In some embodiments, the kit further comprises a plurality of first primer molecules, wherein first primer molecules of said plurality of first primer molecules comprise different first sample identification sequences, wherein the plurality of first primer molecules comprises the first primer molecule. In some embodiments, the plurality of first primer molecules comprises at least 96 first sample identification sequences. In some embodiments, the kit further comprises a plurality of second primer molecules, wherein second primer molecules of said plurality of second primer molecules comprise a different second sample identification sequences, wherein the plurality of second primer molecules comprises the second sample identification sequences. In some embodiments, the plurality of second primer molecules comprises at least 96 second sample identification sequences.

In some embodiments, the kit further comprises a record comprising (i) the plurality of first sample identification sequences and (ii) the plurality of second sample identification sequences. In some embodiments, the kit further comprises instructions to access a record comprising (i) the plurality of first sample identification sequences and (ii) the plurality of second sample identification sequences. In some embodiments, the kit further comprises a computer readable medium comprising instructions in memory configured to present to an operator an interface to record a plurality of pairs of a given first primer identification sequence of the plurality of first sample identification sequences and a given second primer identification sequence of the plurality of second sample identification sequences. In some embodiments, the kit further comprises a transposase. In some embodiments, the kit further comprises an emulsion reagent. In some embodiments, the emulsion reagent comprises a surfactant. In some embodiments, the kit further comprises reagents configured to permeabilize or fix a cell or cell nucleus.

Disclosed herein, in some embodiments, are methods. In an aspect, a method comprises (a) contacting a molecule comprising a tagged deoxyribonucleic acid (DNA) fragment sequence from a cell or cell nucleus of a sample of the plurality of samples, or complement thereof, and a first tag sequence, with a nucleic acid barcode molecule comprising, from 5′ to 3′: (i) a first primer binding sequence, (ii) a barcode sequence, and (iii) first tag binding sequence complementary to the first tag sequence, to generate a first barcoded molecule, wherein the first barcoded molecule comprises a barcode sequence; (b) coupling a first sample identification sequence and a second sample identification sequence to the first barcoded molecule, or derivative thereof.

In some embodiments, the tagged DNA fragment sequence in the molecule is flanked on each side by the first tag sequence and a second tag sequence. In some embodiments, the nucleic acid barcode molecule is coupled to a support. In some embodiments, the nucleic acid barcode molecule is coupled to the support at a 5′ end of the nucleic acid molecule. In some embodiments, the support is a bead. In some embodiments, the bead is a gel bead. In some embodiments, the support is coupled to a plurality of nucleic acid barcode molecules.

In some embodiments, the plurality of nucleic acid molecules comprises the barcode sequence. In some embodiments, a nucleic acid barcode molecule of the plurality of nucleic acid molecules comprises a unique molecular identification sequence that is different from that of another nucleic acid barcode molecule of the plurality of nucleic acid molecules. In some embodiments, the nucleic acid molecule is releasably coupled to the support via a labile moiety. In some embodiments, the kit further comprises cleaving the labile moiety by applying one or more stimuli selected from the group consisting of a thermal, chemical, and photo stimulus. In some embodiments, the labile moiety comprises a disulfide bond.

In some embodiments, the first primer sequence of the first primer molecule does not hybridize to the first tag binding sequence of the nucleic acid barcode molecule. In some embodiments, the first primer binding sequence of the first barcoded molecule does not comprise the first tag binding sequence of the nucleic acid molecule. In some embodiments, the first primer binding sequence of the first barcoded molecule does not correspond to an R1 or R2 sequence. In some embodiments, the first primer binding sequence of the first barcoded molecule does not correspond to a flow cell adapter sequence.

In some embodiments, wherein (b) comprises contacting the first barcoded molecule, or derivative thereof, with (A) a first primer molecule comprising, from 5′ to 3′: (i) the first sample identification sequence and (ii) a first primer sequence corresponding to the first primer binding sequence and (B) a second primer molecule comprising the second sample identification sequence different from the first sample identification sequence. In some embodiments, the first primer molecule comprises a first adapter sequence. In some embodiments, the first adapter sequence is at a 5′ end of the first primer molecule. In some embodiments, the first adapter sequence is configured to attach to a flow cell of a sequencer. In some embodiments, the second primer molecule comprises, from 3′ to 5′: (i) a second tag binding sequence complementary to the second tag sequence, (ii) the second sample identification sequence, and (iii) a second adapter sequence. In some embodiments, the coupling comprises utilizing the first and second primer molecules in an extension reaction. In some embodiments, the extension reaction comprises a PCR amplification reaction.

In some embodiments, wherein (a) comprises partitioning the molecule and the nucleic acid molecule in a partition of a plurality of partitions. In some embodiments, the plurality of partitions comprises a plurality of droplets. In some embodiments, the plurality of partitions comprises a plurality of wells. In some embodiments, the barcode sequence is unique to the partition of the plurality of partitions.

In some embodiments, the method further comprises, prior to (a), generating the tagged DNA fragment sequence from the cell or cell nucleus of the sample. In some embodiments, the generation of the tagged DNA fragment sequence from the cell or cell nucleus of the sample is performed in bulk. In some embodiments, the generation of the tagged DNA fragment sequence from the cell or cell nucleus of the sample is not performed in partition.

In some embodiments, the method further comprises using the barcode sequence to identify the transposed genomic nucleic acid sequence with the cell or cell nucleus from a plurality of cell or cell nuclei. In some embodiments, the method further comprises using a combination of the first sample identification sequence and the second sample identification sequence to identify the tagged DNA fragment sequence with the sample from the plurality of samples.

In some embodiments, wherein (a) comprises barcoding a plurality of tagged DNA fragment sequences, or complements thereof, from a plurality of cells or cell nuclei of the plurality of samples, to generate first barcoded molecules, wherein a first barcoded molecule of the first barcoded molecules comprises a given barcode sequence unique to a given cell or cell nucleus of the plurality of cells or cell nuclei, and (b) comprises coupling a combination of a given first sample identification sequence of a plurality of first sample identification sequences and a given second sample identification sequence of a plurality of second sample identification sequences to the first barcoded molecule, or derivative thereof, wherein the combination is unique to a given sample of the plurality of samples. In some embodiments, the method further comprises using the given barcode sequence to identify the tagged DNA fragment sequence with the cell or cell nucleus from the plurality of cells or cell nuclei. In some embodiments, the method further comprises using the combination of the first sample identification sequence and the second sample identification sequence to identify the tagged DNA fragment sequence with the sample from the plurality of samples. In some embodiments, the tagged DNA fragment sequence comprises a transposed genomic DNA sequence. In some embodiments, the method generates a plurality of dual-indexed nucleic acid molecules combined to generate a sequencing library. In some embodiments, the method further comprises sequencing the sequencing library by generating for a dual-indexed nucleic acid molecule: a first sequencing read comprising the first sample identification sequence and the barcode sequence or complements thereof; a second sequencing read comprising the second sample identification sequence or a complement thereof; a third sequencing read comprising the tagged DNA fragment sequence or complement a thereof, and a fourth sequencing comprising the tagged DNA fragment sequence or complement a thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example of a microfluidic channel structure for partitioning individual analyte carriers.

FIG. 2 shows an example of a microfluidic channel structure for the controlled partitioning of beads into discrete droplets.

FIG. 3 illustrates an example of a barcode carrying bead.

FIG. 4 illustrates another example of a barcode carrying bead.

FIG. 5 schematically illustrates an example microwell array.

FIG. 6 schematically illustrates an example workflow for processing nucleic acid molecules.

FIG. 7 shows a computer system that is programmed or otherwise configured to implement methods provided herein.

FIG. 8 shows beads for use according to the methods of the present disclosure.

FIG. 9 illustrates a transposase-nucleic acid complex comprising a transposase, a first double-stranded oligonucleotide comprising a transposon end sequence and a first sequencing primer sequence and a second double-stranded oligonucleotide comprising a transposon end sequence and a second sequencing primer sequence.

FIG. 10 illustrates a transposase-nucleic acid complex comprising a transposase, a first double-stranded oligonucleotide comprising a transposon end sequence and first and second sequencing primer sequences and a second double-stranded oligonucleotide comprising a transposon end sequence and third and fourth sequencing primer sequences.

FIG. 11 illustrates a transposase-nucleic acid complex comprising a transposase, a first hairpin molecule, and a second hairpin molecule.

FIG. 12 illustrates a scheme for dual indexing and barcoding an ATAC sample.

FIG. 13 illustrates an additional scheme for dual indexing and barcoding an ATAC sample.

FIG. 14 illustrates two dual-indexed and barcoded nucleic acid molecules.

FIG. 15 illustrates three types of labelling agents attached with reporter oligonucleotides.

FIGS. 16A-C illustrate multiple bead-attached oligonucleotides. FIG. 16A schematically shows an example of labelling agents. FIG. 16B schematically shows another example workflow for processing nucleic acid molecules. FIG. 16C schematically shows another example workflow for processing nucleic acid molecules.

FIG. 17 illustrates multiple primers for dual indexing nucleic acid molecules.

FIGS. 18A-D illustrate four barnyard plots of barcoded mouse sequence reads (y-axis) and human sequence reads (x-axis).

FIGS. 19A-D illustrate four single-cell targeting plots for assessing sample assignment.

FIG. 20 schematically shows another example of a barcode-carrying bead.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The terms “a,” “an,” and “the,” as used herein, generally refers to singular and plural references unless the context clearly dictates otherwise.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “barcode,” as used herein, generally refers to a label, or identifier, that conveys or is capable of conveying information about an analyte. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include: polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads.

The term “real time,” as used herein, can refer to a response time of less than about 1 second, a tenth of a second, a hundredth of a second, a millisecond, or less. The response time may be greater than 1 second. In some instances, real time can refer to simultaneous or substantially simultaneous processing, detection or identification.

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses).

The term “genome,” as used herein, generally refers to genomic information from a subject, which may be, for example, at least a portion or an entirety of a subject's hereditary information. A genome can be encoded either in DNA or in RNA. A genome can comprise coding regions (e.g., that code for proteins) as well as non-coding regions. A genome can include the sequence of all chromosomes together in an organism. For example, the human genome ordinarily has a total of 46 chromosomes. The sequence of all of these together may constitute a human genome.

The terms “adaptor(s)”, “adapter(s)” and “tag(s)” may be used synonymously. An adaptor or tag can be coupled to a polynucleotide sequence to be “tagged” by any approach, including ligation, hybridization, or other approaches.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

As used herein, the term “barcoded nucleic acid molecule” generally refers to a nucleic acid molecule that results from, for example, the processing of a nucleic acid barcode molecule with a nucleic acid sequence. The nucleic acid sequence may be a targeted sequence or a non-targeted sequence. The nucleic acid barcode molecule may be coupled to or attached to the nucleic acid molecule comprising the nucleic acid sequence. For example, in the methods and systems described herein, hybridization and reverse transcription of a nucleic acid molecule (e.g., a messenger RNA (mRNA) molecule) of a cell with a nucleic acid barcode molecule (e.g., a nucleic acid barcode molecule containing a barcode sequence and a nucleic acid primer sequence complementary to a nucleic acid sequence of the mRNA molecule) results in a barcoded nucleic acid molecule that has a sequence corresponding to the nucleic acid sequence of the mRNA and the barcode sequence (or a reverse complement thereof). The processing of the nucleic acid molecule comprising the nucleic acid sequence, the nucleic acid barcode molecule, or both, can include a nucleic acid reaction, such as, in non-limiting examples, reverse transcription, nucleic acid extension, ligation, etc. The nucleic acid reaction may be performed prior to, during, or following barcoding of the nucleic acid sequence to generate the barcoded nucleic acid molecule. For example, the nucleic acid molecule comprising the nucleic acid sequence may be subjected to reverse transcription and then be attached to the nucleic acid barcode molecule to generate the barcoded nucleic acid molecule, or the nucleic acid molecule comprising the nucleic acid sequence may be attached to the nucleic acid barcode molecule and subjected to a nucleic acid reaction (e.g., extension, ligation) to generate the barcoded nucleic acid molecule. A barcoded nucleic acid molecule may serve as a template, such as a template polynucleotide, that can be further processed (e.g., amplified) and sequenced to obtain the target nucleic acid sequence. For example, in the methods and systems described herein, a barcoded nucleic acid molecule may be further processed (e.g., amplified) and sequenced to obtain the nucleic acid sequence of the nucleic acid molecule.

The term “sample,” as used herein, generally refers to a biological sample of a subject. The biological sample may comprise any number of macromolecules, for example, cellular macromolecules. The sample may be a cell sample. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

The term “biological particle,” as used herein, generally refers to a discrete biological system derived from a biological sample. The biological particle may be a macromolecule. The biological particle may be a small molecule. The biological particle may be a virus. The biological particle may be a cell or derivative of a cell. The biological particle may be an organelle. The biological particle may be a rare cell from a population of cells. The biological particle may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle may be a constituent of a cell. The biological particle may be or may include DNA, RNA, organelles, proteins, or any combination thereof. The biological particle may be or may include a matrix (e.g., a gel or polymer matrix) comprising a cell or one or more constituents from a cell (e.g., cell bead), such as DNA, RNA, organelles, proteins, or any combination thereof, from the cell. The biological particle may be obtained from a tissue of a subject. The biological particle may be a hardened cell. Such hardened cell may or may not include a cell wall or cell membrane. The biological particle may include one or more constituents of a cell, but may not include other constituents of the cell. An example of such constituents is a nucleus or an organelle. A cell may be a live cell. The live cell may be capable of being cultured, for example, being cultured when enclosed in a gel or polymer matrix, or cultured when comprising a gel or polymer matrix.

The term “macromolecular constituent,” as used herein, generally refers to a macromolecule contained within or from a biological particle. The macromolecular constituent may comprise a nucleic acid. In some cases, the biological particle may be a macromolecule. The macromolecular constituent may comprise DNA. The macromolecular constituent may comprise RNA. The RNA may be coding or non-coding. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (TRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA. The macromolecular constituent may comprise a protein. The macromolecular constituent may comprise a peptide. The macromolecular constituent may comprise a polypeptide.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

The term “partition,” as used herein, generally, refers to a space or volume that may be suitable to contain one or more species or conduct one or more reactions. A partition may be a physical compartment, such as a droplet or well. The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

The present disclosure provides kits, compositions, systems, and methods for dual indexing nucleic acid molecules. The kits, compositions, systems, and methods provided herein may facilitate multiplexed sample preparation, nucleic acid molecule processing, sequencing library generation, and sequencing analysis of nucleic acid molecules included in or from cells, cell beads, or cell nuclei of interest. For example, the present disclosure provides methods for processing deoxyribonucleic acid (DNA) molecules included within a cell, cell bead, or cell nucleus. The methods may comprise performing Assay for Transposase Accessible Chromatin with high-throughput sequencing (ATAC-seq). Partitioning and barcoding schemes as described herein may be utilized to facilitate identification of resultant sequencing reads with the cell, cell bead, or cell nucleus from which they are derived. In some cases, errors in identifying the origin of a sequenced nucleic acid may occur due to a phenomenon known as index hopping. In some examples, index hopping may occur when pooled samples are sequenced to high depth. Index hopping may occur if index tag sequences from one library are inadvertently added to a nucleic acid from a different library. In some aspects, index hopping may occur during library preparation or cluster amplification of the polynucleotides on a flow cell or other support used for sequencing. In some cases, index hopping may confound results of sequencing, such as resulting in misassignment of library origin of a sequenced nucleic acid molecule, filtering or discarding sequencing results. Dual indexing may be utilized to facilitate correct assignment of sequencing reads with the originating sample. In some embodiments, dual indexing comprises using unique and distinct sequences (e.g., a first and second sample identification sequence) for each of the index reads (e.g., i5 and i7).

### Dual Indexing and DNA Barcoding

Provided herein are methods, systems, kits, and compositions for processing a nucleic acid molecule including dual indexing and barcoding. Provided herein are methods, systems, kits, and compositions for preparing a sequencing library that includes nucleic acids (e.g., tagged deoxyribonucleic acid (DNA) fragments) from biological particles (e.g., cells or cell nuclei). A barcode sequence may comprise a first nucleic acid sequence that can identify or distinguish a second nucleic acid molecule or sequence from one or more other nucleic acid molecules or sequences. In some cases, a barcode sequence may comprise a first nucleic acid sequence that can identify or distinguish a first set of nucleic acid molecules or sequences from one or more from other sets of nucleic acid molecules or sequences. In some cases, a combination of a plurality of barcode sequences can identify or distinguish a second nucleic acid molecule or sequence from one or more other nucleic acid molecules or sequences. In some cases, a combination of a plurality of barcode sequences can identify or distinguish a first set of nucleic acid molecules or sequences from one or more other sets of nucleic acid molecules or sequences. As described elsewhere herein, a barcode may identify and distinguish a first set of nucleic acid molecules derived from a first biological particle (e.g., cell or cell nucleus) from a second set of nucleic acid molecules derived from a second biological particle (e.g., cell or cell nucleus).

A sample identification sequence can be or comprise an index sequence. A sample identification sequence may comprise a nucleic acid sequence that can identify or distinguish an originating sample from one or more other samples. In some cases, a sample identification sequence may comprise a nucleic acid sequence that can identify or distinguish a set of originating samples from one or more other sets of samples. In some cases, a combination of sample identification sequences can identify or distinguish an originating sample from one or more other samples. In some cases, a combination of sample identification sequences can identify or distinguish a first set of originating samples from other sets of samples. As described elsewhere herein, a combination of two sample identification sequences may identify and distinguish a first set of nucleic acid molecules derived from a first originating sample from a second set of nucleic acid molecules derived from a second originating sample.

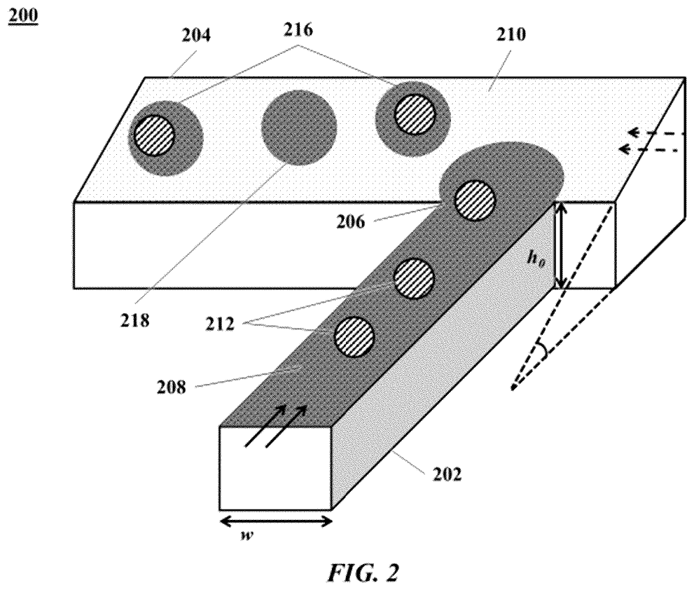

In some cases, barcoding and/or indexing with barcode and/or sample identification sequence(s), respectively, is performed by co-partitioning the individual biological particle (e.g., cell, cell nucleus, cell bead, etc.) or groups of biological particles with the barcode and/or sample identification sequence(s) in individual partitions, such as described elsewhere herein (e.g., with reference to FIG. 2). In other cases, a barcode and/or sample identification sequence may be provided to a group of nucleic acid molecules in bulk solution. In some cases, the barcode and/or sample identification sequences are provided in the form of nucleic acid molecules (e.g., oligonucleotides) comprising barcode and/or sample identification sequences that may be attached to or otherwise associated with the nucleic acid contents (e.g., DNA molecules, fragments thereof) or other components of an individual biological particle (e.g., cell, cell nucleus, cell bead, etc.), or derivative thereof. Nucleic acid barcode molecules may be partitioned such that between nucleic acid barcode molecules in a given partition, the respective nucleic acid barcode sequences contained therein are the same, but between different partitions, the nucleic acid barcode molecules have differing barcode sequences, or at least represent a large number of different barcode sequences across all of the partitions in a given analysis. In such cases, the nucleic acid barcode molecules may further comprise an additional barcode sequence, such as a unique molecular identifier sequence, which varies across nucleic acid barcode molecules even within the same partition. Nucleic acid barcode molecules having the common partition sequence may be coupled to a bead, and the bead may be delivered to the partition. In some cases, only one nucleic acid barcode sequence can be associated with a given partition, although in some cases, two or more different barcode sequences may be present.

Nucleic acid sample identification sequences may be delivered such that between nucleic acid molecules derived from a same originating sample, the nucleic acid sample identification sequences are the same, but between different samples, the nucleic acid molecules have differing sample identification sequences, or at least represent a large number of different sample identification sequences across all of the samples in a given analysis. In some cases, a sample identification sequence may identify a first set of nucleic acid molecules (e.g., to an originating sample) and a barcode sequence may identify a first subset of nucleic acid molecules in the first set of nucleic acid molecules (e.g., to a cell, cell nucleus, or cell bead of the originating sample). In other cases, a pair of sample identification sequences may identify a first set of nucleic acid molecules (e.g., to an originating sample) and a barcode sequence may identify a first subset of nucleic acid molecules in the first set of nucleic acid molecules (e.g., to a cell, cell nucleus, or cell bead of the originating sample).

In some cases, a first nucleic acid sample identification sequence and a second nucleic acid sample identification sequence may combine to uniquely identify a sample or a group of samples. In other cases, a first nucleic acid sample identification sequence may only identify a sample or a group of samples when a second nucleic acid sample identification sequence is present on the nucleic acid molecule. In other cases, any number of sample identification sequences may combine to identify a sample or a group of samples. In some cases, a first nucleic acid sample identification sequence and a second nucleic acid sample identification sequence may combine with a nucleic barcode sequence to identify a unique nucleic acid molecule or a group of unique nucleic acid molecules.

In some cases, the identity of a sample identification sequence of a plurality of sample identification sequences, assigned to a sample from a plurality of samples in an experiment or analysis, is known and/or recorded. In other cases, the identity of a first and second sample identification sequence of a plurality of sample identification sequences, assigned to a sample from a plurality of samples in an experiment or analysis, is known and/or recorded. In other cases, the identity of a sample identification sequence of a plurality of sample identification sequences assigned to a sample from a plurality of samples in an experiment or analysis is known and/or recorded before the experiment or analysis is carried out. An experiment can comprise processing nucleic acid molecules from a sample, generating a sequencing library, and/or performing a multiplex sequencing analysis. A multiplex sequencing analysis can comprise a sequencing analysis of more than one sample. The multiplex sequencing analysis may comprise one or more operations in which more than one sample is processed in parallel and/or in bulk.

In some cases, a multiplex sequencing analysis can comprise processing about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or more samples. In some cases, a multiplex sequencing analysis can comprise processing about 10×10, 11×11, 12×12, 13×13, 14×14, 15×15, 16×16, 17×17, 18×18, 19×19, 20×20, 21×21, 22×22, 23×23, 24×24, 25×25, 26×26, 27×27, 28×28, 29×29, 30×30, 31×31, 32×32, 33×33, 34×34, 35×35, 36×36, 37×37, 38×38, 39×39, 40×40, 41×41, 42×42, 43×43, 44×44, 45×45, 46×46, 47×47, 48×48, 49×49, 50×50, 51×51, 52×52, 53×53, 54×54, 55×55, 56×56, 57×57, 58×58, 59×59, 60×60, 61×61, 62×62, 63×63, 64×64, 65×65, 66×66, 67×67, 68×68, 69×69, 70×70, 71×71, 72×72, 73×73, 74×74, 75×75, 76×76, 77×77, 78×78, 79×79, 80×80, 81×81, 82×82, 83×83, 84×84, 85×85, 86×86, 87×87, 88×88, 89×89, 90×90, 91×91, 92×92, 93×93, 94×94, 95×95, 96×96, 97×97, 98×98, 99×99, 100×100, 101×101, 102×102, 103×103, 104×104, 105×105, 106×106, 107×107, 108×108, 109×109, 110×110, 111×111, 112×112, 113×113, 114×114, 115×115, 116×116, 117×117, 118×118, 119×119, 120×120, 121×121, 122×122, 123×123, 124×124, 125×125, 126×126, 127×127, 128×128, 129×129, 130×130, 131×131, 132×132, 133×133, 134×134, 135×135, 136×136, 137×137, 138×138, 139×139, 140×140, 141×141, 142×142, 143×143, 144×144, 145×145, 146×146, 147×147, 148×148, 149×149, 150×150, 151×151, 152×152, 153×153, 154×154, 155×155, 156×156, 157×157, 158×158, 159×159, 160×160, 161×161, 162×162, 163×163, 164×164, 165×165, 166×166, 167×167, 168×168, 169×169, 170×170, 171×171, 172×172, 173×173, 174×174, 175×175, 176×176, 177×177, 178×178, 179×179, 180×180, 181×181, 182×182, 183×183, 184×184, 185×185, 186×186, 187×187, 188×188, 189×189, 190×190, 191×191, 192×192, 193×193, 194×194, 195×195, 196×196, 197×197, 198×198, 199×199, 200×200, 201×201, 202×202, 203×203, 204×204, 205×205, 206×206, 207×207, 208×208, 209×209, 210×210, 211×211, 212×212, 213×213, 214×214, 215×215, 216×216, 217×217, 218×218, 219×219, 220×220, 221×221, 222×222, 223×223, 224×224, 225×225, 226×226, 227×227, 228×228, 229×229, 230×230, 231×231, 232×232, 233×233, 234×234, 235×235, 236×236, 237×237, 238×238, 239×239, 240×240, 241×241, 242×242, 243×243, 244×244, 245×245, 246×246, 247×247, 248×248, 249×249, 250×250, 251×251, 252×252, 253×253, 254×254, 255×255, 256×256, 257×257, 258×258, 259×259, 260×260, 261×261, 262×262, 263×263, 264×264, 265×265, 266×266, 267×267, 268×268, 269×269, 270×270, 271×271, 272×272, 273×273, 274×274, 275×275, 276×276, 277×277, 278×278, 279×279, 280×280, 281×281, 282×282, 283×283, 284×284, 285×285, 286×286, 287×287, 288×288, 289×289, 290×290, 291×291, 292×292, 293×293, 294×294, 295×295, 296×296, 297×297, 298×298, 299×299, 300×300, 301×301, 302×302, 303×303, 304×304, 305×305, 306×306, 307×307, 308×308, 309×309, 310×310, 311×311, 312×312, 313×313, 314×314, 315×315, 316×316, 317×317, 318×318, 319×319, 320×320, 321×321, 322×322, 323×323, 324×324, 325×325, 326×326, 327×327, 328×328, 329×329, 330×330, 331×331, 332×332, 333×333, 334×334, 335×335, 336×336, 337×337, 338×338, 339×339, 340×340, 341×341, 342×342, 343×343, 344×344, 345×345, 346×346, 347×347, 348×348, 349×349, 350×350, 351×351, 352×352, 353×353, 354×354, 355×355, 356×356, 357×357, 358×358, 359×359, 360×360, 361×361, 362×362, 363×363, 364×364, 365×365, 366×366, 367×367, 368×368, 369×369, 370×370, 371×371, 372×372, 373×373, 374×374, 375×375, 376×376, 377×377, 378×378, 379×379, 380×380, 381×381, 382×382, 383×383, 384×384, 385×385, 386×386, 387×387, 388×388, 389×389, 390×390, 391×391, 392×392, 393×393, 394×394, 395×395, 396×396, 397×397, 398×398, 399×399, 400×400, 401×401, 402×402, 403×403, 404×404, 405×405, 406×406, 407×407, 408×408, 409×409, 410×410, 411×411, 412×412, 413×413, 414×414, 415×415, 416×416, 417×417, 418×418, 419×419, 420×420, 421×421, 422×422, 423×423, 424×424, 425×425, 426×426, 427×427, 428×428, 429×429, 430×430, 431×431, 432×432, 433×433, 434×434, 435×435, 436×436, 437×437, 438×438, 439×439, 440×440, 441×441, 442×442, 443×443, 444×444, 445×445, 446×446, 447×447, 448×448, 449×449, 450×450, 451×451, 452×452, 453×453, 454×454, 455×455, 456×456, 457×457, 458×458, 459×459, 460×460, 461×461, 462×462, 463×463, 464×464, 465×465, 466×466, 467×467, 468×468, 469×469, 470×470, 471×471, 472×472, 473×473, 474×474, 475×475, 476×476, 477×477, 478×478, 479×479, 480×480, 481×481, 482×482, 483×483, 484×484, 485×485, 486×486, 487×487, 488×488, 489×489, 490×490, 491×491, 492×492, 493×493, 494×494, 495×495, 496×496, 497×497, 498×498, 499×499, 500×500, or more samples. In some cases, a multiplex sequencing analysis can comprise processing from 1×10\hat{ }0 to 1×10\hat{ }2, from 1×10\hat{ }2 to 5×10\hat{ }3, from 1×10\hat{ }3 to 5×10\hat{ }4, from 1×10\hat{ }4 to 5×10\hat{ }5, from 1×10\hat{ }5 to 5×10\hat{ }6, from 1×10\hat{ }6 to 5×10\hat{ }7, from 1×10\hat{ }7 to 5×10\hat{ }8, from 1×10\hat{ }8 to 5×10\hat{ }9, from 1×10\hat{ }9 to 5×10\hat{ }10, from 1×10\hat{ }10 to 5×10\hat{ }11, from 1×10\hat{ }11 to 5×10\hat{ }12, from 1×10\hat{ }12 to 5×10\hat{ }13, from 1×10\hat{ }13 to 5×10\hat{ }14, from 1×10\hat{ }14 to 5×10\hat{ }15, from 1×10\hat{ }15 to 5×10\hat{ }16, from 1×10\hat{ }16 to 5×10\hat{ }17, from 1×10\hat{ }17 to 5×10\hat{ }18, from 1×10\hat{ }18 to 5×10\hat{ }19, from 1×10\hat{ }19 to 5×10\hat{ }20, or more samples. In other cases, a multiplex sequencing analysis can comprise processing about 1000 samples. In other cases, a multiplex sequencing analysis can comprise processing about 5000 samples. In other cases, a multiplex sequencing analysis can comprise processing about 9000 samples.

A sample can comprise a cell or a group of biological particles (e.g., cells or cell nuclei). A group of biological particles (e.g., cells or cell nuclei) can comprise a plurality of biological particles undergoing a same treatment, manipulation, or procedure. In other cases, a group of biological particles can be defined by a plurality of biological particles sharing a same origin, protein marker, nucleic acid marker, chemical marker, disease state, age, function, location, physical/chemical/biological activity, genotype, phenotype, or any combinations thereof. In other cases, a group of biological particles can be arbitrarily assigned, assembled, categorized, ordered, classified, organized, or ranked.

In some cases, an experiment or analysis of sequencing experiments can comprise a plurality of sequencing reads from multiple samples. Sample identification sequences can assign the sequencing reads to their original samples, or otherwise identify sequencing reads as originating from the same sample. In some cases, sets (e.g., pairs) of sample identification sequences may be assigned to specific different samples. In one example, a first set of first and second sample identification sequences are assigned to a first sample and, a second set of first and second sample identification sequences are assigned to a second sample. A first sequencing read associated with the first and second sample identification sequences from the first set can identify the first sequencing read as having originated from the first sample. A second sequencing read associated with the first and second sample identification sequences from the second set can identify the second sequencing read as having originated from the second sample. A third sequencing read associated with the first sample identification sequence from the first set and the second sample identification sequence not from the first set, or the first sample identification sequence from the second set and the second sample identification sequence not from the second set may not identify the third sequencing read as having originated from any known sample. In some cases, a sequencing read without an assignment of a sample may be filtered out, discarded or removed from further experiment or analysis. In some cases, sequencing reads associated with the same set of sample identification sequences, without an assignment to a specific sample, may be categorized as its own sample. In other cases, sets of sample identification sequences may be recorded as being assigned to different samples, though not to specific samples. Sequencing reads comprising or associated with the same set of sample identification sequences may be identified as originating from the same sample. In some cases, mismatched assignment of sample identification sequences can be identified, filtered out, and/or discarded to prevent confounding results or impacting accuracy of the sequencing. In some aspects, a pair of sample identification sequences from the sequencing read(s) can be compared to the sample identification sequences that were assigned to a sample, which was known or recorded. In some particular embodiments, an unexpected pairing or association of sample identification sequences can be identified.

A sequencing experiment can comprise about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, or more sets of sample identification sequences. In some instances, a sequencing experiment can comprise from 1 to 10, from 5 to 15, from 10 to 20, from 15 to 25, from 20 to 30, from 25 to 35, from 30 to 40, from 35 to 45, from 40 to 50, from 45 to 55, from 50 to 60, from 55 to 65, from 60 to 70, from 65 to 75, from 70 to 80, from 75 to 85, from 80 to 90, from 85 to 95, from 90 to 100, from 95 to 105, from 100 to 110, from 105 to 115, from 110 to 120, from 115 to 125, from 120 to 130, from 125 to 135, from 130 to 140, from 135 to 145, from 140 to 150, from 145 to 155, from 150 to 160, from 155 to 165, from 160 to 170, from 165 to 175, from 170 to 180, from 175 to 185, from 180 to 190, from 185 to 195, from 190 to 200, from 195 to 205, from 200 to 210, from 205 to 215, from 210 to 220, from 215 to 225, from 220 to 230, from 225 to 235, from 230 to 240, from 235 to 245, from 240 to 250, from 245 to 255, from 250 to 260, from 255 to 265, from 260 to 270, from 265 to 275, from 270 to 280, from 275 to 285, from 280 to 290, from 285 to 295, from 290 to 300, from 295 to 305, from 300 to 310, from 305 to 315, from 310 to 320, from 315 to 325, from 320 to 330, from 325 to 335, from 330 to 340, from 335 to 345, from 340 to 350, from 345 to 355, from 350 to 360, from 355 to 365, from 360 to 370, from 365 to 375, from 370 to 380, from 375 to 385, from 380 to 390, from 385 to 395, from 390 to 400, from 395 to 405, from 400 to 410, from 405 to 415, from 410 to 420, from 415 to 425, from 420 to 430, from 425 to 435, from 430 to 440, from 435 to 445, from 440 to 450, from 445 to 455, from 450 to 460, from 455 to 465, from 460 to 470, from 465 to 475, from 470 to 480, from 475 to 485, from 480 to 490, from 485 to 495, from 490 to 500, or more sets of sample identification sequences. In some cases, a sequencing experiment can comprise 96 sets of sample identification sequences.

A set of sample identification sequences can comprise about 1, 2, 3, 4, 5, 6, 7, 8, 9, 10, or more sample identification sequences. For dual indexing schemes as described herein, a set of sample identification sequences can comprise 2 sample identification sequences.

A nucleic acid barcode sequence can comprise about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more nucleotides within the sequence of the nucleic acid molecules (e.g., oligonucleotides). The nucleic acid barcode sequences can comprise from 1 to 10, from 2 to 11, from 3 to 12, from 4 to 13, from 5 to 14, from 6 to 15, from 7 to 16, from 8 to 17, from 9 to 18, from 10 to 19, from 11 to 20, from 12 to 21, from 13 to 22, from 14 to 23, from 15 to 24, from 16 to 25, from 17 to 26, from 18 to 27, from 19 to 28, from 20 to 29, from 21 to 30, from 22 to 31, from 23 to 32, from 24 to 33, from 25 to 34, from 26 to 35, from 27 to 36, from 28 to 37, from 29 to 38, from 30 to 39, from 31 to 40, from 32 to 41, from 33 to 42, from 34 to 43, from 35 to 44, from 36 to 45, from 37 to 46, from 38 to 47, from 39 to 48, from 40 to 49, from 41 to 50, from 42 to 51, from 43 to 52, from 44 to 53, from 45 to 54, from 46 to 55, from 47 to 56, from 48 to 57, from 49 to 58, from 50 to 59, from 51 to 60, from 52 to 61, from 53 to 62, from 54 to 63, from 55 to 64, from 56 to 65, from 57 to 66, from 58 to 67, from 59 to 68, from 60 to 69, from 61 to 70, from 62 to 71, from 63 to 72, from 64 to 73, from 65 to 74, from 66 to 75, from 67 to 76, from 68 to 77, from 69 to 78, from 70 to 79, from 71 to 80, from 72 to 81, from 73 to 82, from 74 to 83, from 75 to 84, from 76 to 85, from 77 to 86, from 78 to 87, from 79 to 88, from 80 to 89, from 81 to 90, from 82 to 91, from 83 to 92, from 84 to 93, from 85 to 94, from 86 to 95, from 87 to 96, from 88 to 97, from 89 to 98, from 90 to 99, or from 91 to 100 nucleotides. In some cases, the length of a barcode sequence may be about 6, 7, 8, 9, 10, 11, 12, 13, 14, 15, 16, 17, 18, 19, 20 nucleotides or longer. In some cases, the length of a barcode sequence may be at least about 6, 7, 8, 9, 10, 11, 12, 13, 14, 15, 16, 17, 18, 19, 20 nucleotides or longer. In some cases, the length of a barcode sequence may be at most about 6, 7, 8, 9, 10, 11, 12, 13, 14, 15, 16, 17, 18, 19, 20 nucleotides or shorter. In some cases, the length of a barcode sequence may be about 16 nucleotides. These nucleotides may be completely contiguous, e.g., in a single stretch of adjacent nucleotides, or they may be separated into two or more separate subsequences that are separated by 1 or more nucleotides.

A nucleic acid barcode sequence can comprise a unique molecular identification (UMI) sequence. A unique molecular identification sequence can comprise about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more nucleotides within the sequence of the nucleic acid molecules (e.g., oligonucleotides). A unique molecular identification sequence can comprise from 1 to 10, from 2 to 11, from 3 to 12, from 4 to 13, from 5 to 14, from 6 to 15, from 7 to 16, from 8 to 17, from 9 to 18, from 10 to 19, from 11 to 20, from 12 to 21, from 13 to 22, from 14 to 23, from 15 to 24, from 16 to 25, from 17 to 26, from 18 to 27, from 19 to 28, from 20 to 29, from 21 to 30, from 22 to 31, from 23 to 32, from 24 to 33, from 25 to 34, from 26 to 35, from 27 to 36, from 28 to 37, from 29 to 38, from 30 to 39, from 31 to 40, from 32 to 41, from 33 to 42, from 34 to 43, from 35 to 44, from 36 to 45, from 37 to 46, from 38 to 47, from 39 to 48, from 40 to 49, from 41 to 50, from 42 to 51, from 43 to 52, from 44 to 53, from 45 to 54, from 46 to 55, from 47 to 56, from 48 to 57, from 49 to 58, from 50 to 59, from 51 to 60, from 52 to 61, from 53 to 62, from 54 to 63, from 55 to 64, from 56 to 65, from 57 to 66, from 58 to 67, from 59 to 68, from 60 to 69, from 61 to 70, from 62 to 71, from 63 to 72, from 64 to 73, from 65 to 74, from 66 to 75, from 67 to 76, from 68 to 77, from 69 to 78, from 70 to 79, from 71 to 80, from 72 to 81, from 73 to 82, from 74 to 83, from 75 to 84, from 76 to 85, from 77 to 86, from 78 to 87, from 79 to 88, from 80 to 89, from 81 to 90, from 82 to 91, from 83 to 92, from 84 to 93, from 85 to 94, from 86 to 95, from 87 to 96, from 88 to 97, from 89 to 98, from 90 to 99, or from 91 to 100 nucleotides. In some cases, the length of a unique molecular identification sequence may be about 6, 7, 8, 9, 10, 11, 12, 13, 14, 15, 16, 17, 18, 19, 20 nucleotides or longer. In some cases, the length of a unique molecular identification sequence may be at least about 6, 7, 8, 9, 10, 11, 12, 13, 14, 15, 16, 17, 18, 19, 20 nucleotides or longer. In some cases, the length of a unique molecular identification sequence may be at most about 6, 7, 8, 9, 10, 11, 12, 13, 14, 15, 16, 17, 18, 19, 20 nucleotides or shorter. In some cases, the length of a unique molecular identification sequence may be about 16 nucleotides. These nucleotides may be completely contiguous, e.g., in a single stretch of adjacent nucleotides, or they may be separated into two or more separate subsequences that are separated by 1 or more nucleotides.

The nucleic acid sample identification sequence can comprise from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more nucleotides. The nucleic acid sample identification sequence can comprise from 1 to 10, from 2 to 11, from 3 to 12, from 4 to 13, from 5 to 14, from 6 to 15, from 7 to 16, from 8 to 17, from 9 to 18, from 10 to 19, from 11 to 20, from 12 to 21, from 13 to 22, from 14 to 23, from 15 to 24, from 16 to 25, from 17 to 26, from 18 to 27, from 19 to 28, from 20 to 29, from 21 to 30, from 22 to 31, from 23 to 32, from 24 to 33, from 25 to 34, from 26 to 35, from 27 to 36, from 28 to 37, from 29 to 38, from 30 to 39, from 31 to 40, from 32 to 41, from 33 to 42, from 34 to 43, from 35 to 44, from 36 to 45, from 37 to 46, from 38 to 47, from 39 to 48, from 40 to 49, from 41 to 50, from 42 to 51, from 43 to 52, from 44 to 53, from 45 to 54, from 46 to 55, from 47 to 56, from 48 to 57, from 49 to 58, from 50 to 59, from 51 to 60, from 52 to 61, from 53 to 62, from 54 to 63, from 55 to 64, from 56 to 65, from 57 to 66, from 58 to 67, from 59 to 68, from 60 to 69, from 61 to 70, from 62 to 71, from 63 to 72, from 64 to 73, from 65 to 74, from 66 to 75, from 67 to 76, from 68 to 77, from 69 to 78, from 70 to 79, from 71 to 80, from 72 to 81, from 73 to 82, from 74 to 83, from 75 to 84, from 76 to 85, from 77 to 86, from 78 to 87, from 79 to 88, from 80 to 89, from 81 to 90, from 82 to 91, from 83 to 92, from 84 to 93, from 85 to 94, from 86 to 95, from 87 to 96, from 88 to 97, from 89 to 98, from 90 to 99, or from 91 to 100 nucleotides. In some cases, the length of a sample identification sequence may be about 2, 3, 4, 5, 6, 7, 8, 9, 10, 11, 12, 13, 14, 15, 16 nucleotides or longer. In some cases, the length of a barcode sequence may be at least about 2, 3, 4, 5, 6, 7, 8, 9, 10, 11, 12, 13, 14, 15, 16 nucleotides or longer. In some cases, the length of a barcode sequence may be at most about 2, 3, 4, 5, 6, 7, 8, 9, 10, 11, 12, 13, 14, 15, 16 nucleotides or shorter. In some cases, the length of a barcode sequence may be about 8 nucleotides. These nucleotides may be completely contiguous, e.g., in a single stretch of adjacent nucleotides, or they may be separated into two or more separate subsequences that are separated by 1 or more nucleotides.

A set of sample identification sequences can comprise (i) a first sample identification sequence from a first group of sample identification sequences and (ii) a second sample identification sequence selected from a second group of sample identification sequences. The first sample identification sequence can have a different nucleotide sequence from that of the second sample identification sequence. A first sample identification sequence can have a different nucleotide length from that of a second sample identification sequence. In some instances, a first and a second sample identification sequence can have the same nucleotide length but different nucleotide sequences. In some cases, a first sample identification sequence can have the same nucleotide sequence as that of a second sample identification sequence. In some instances, a first and a second sample identification sequence can have a difference of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more nucleotides. A first and a second sample identification sequence can also have a difference from 1 to 10, from 2 to 11, from 3 to 12, from 4 to 13, from 5 to 14, from 6 to 15, from 7 to 16, from 8 to 17, from 9 to 18, from 10 to 19, from 11 to 20, from 12 to 21, from 13 to 22, from 14 to 23, from 15 to 24, from 16 to 25, from 17 to 26, from 18 to 27, from 19 to 28, from 20 to 29, from 21 to 30, from 22 to 31, from 23 to 32, from 24 to 33, from 25 to 34, from 26 to 35, from 27 to 36, from 28 to 37, from 29 to 38, from 30 to 39, from 31 to 40, from 32 to 41, from 33 to 42, from 34 to 43, from 35 to 44, from 36 to 45, from 37 to 46, from 38 to 47, from 39 to 48, from 40 to 49, from 41 to 50, from 42 to 51, from 43 to 52, from 44 to 53, from 45 to 54, from 46 to 55, from 47 to 56, from 48 to 57, from 49 to 58, from 50 to 59, from 51 to 60, from 52 to 61, from 53 to 62, from 54 to 63, from 55 to 64, from 56 to 65, from 57 to 66, from 58 to 67, from 59 to 68, from 60 to 69, from 61 to 70, from 62 to 71, from 63 to 72, from 64 to 73, from 65 to 74, from 66 to 75, from 67 to 76, from 68 to 77, from 69 to 78, from 70 to 79, from 71 to 80, from 72 to 81, from 73 to 82, from 74 to 83, from 75 to 84, from 76 to 85, from 77 to 86, from 78 to 87, from 79 to 88, from 80 to 89, from 81 to 90, from 82 to 91, from 83 to 92, from 84 to 93, from 85 to 94, from 86 to 95, from 87 to 96, from 88 to 97, from 89 to 98, from 90 to 99, or from 91 to 100 nucleotides. A first sample and a second sample identification sequence can also have a difference from 1 to 2, from 1 to 3, from 1 to 4, from 1 to 5, from 1 to 6, from 1 to 7, or from 1 to 8 nucleotides.

The present disclosure provides a method for processing nucleic acid molecules from a cell, cell bead, or cell nucleus. The method may comprise contacting a cell, cell bead, or cell nucleus with a transposase-nucleic acid complex comprising a transposase molecule and one or more transposon end oligonucleotide molecules. The cell, cell bead or cell nucleus may be contacted with a transposase-nucleic acid complex in bulk solution, such that the cell, cell bead or cell nucleus undergoes “tagmentation” via a tagmentation reaction (e.g., fragmenting and tagging). Contacting the cell, cell bead, or cell nucleus with the transposase-nucleic acid complex may generate one or more template nucleic acid fragments that are flanked by tag sequences (e.g., “tagmented fragments” or “tagged fragments”). The one or more template nucleic acid fragments may correspond to one or more target or template nucleic acid molecules (e.g., deoxyribonucleic acid (DNA) molecules) within the cell, cell bead, or cell nucleus. The cell, cell bead, or cell nucleus comprising the one or more template nucleic acid fragments may be partitioned (e.g., co-partitioned with one or more reagents) into a partition (e.g., of a plurality of partitions). The partition may be, for example, a droplet or a well. The partition may comprise one or more reagents, including, for example, one or more particles (e.g., beads) comprising one or more nucleic acid barcode molecules comprising barcode sequences. The cell, cell bead, or cell nucleus may be lysed, permeabilized, fixed, cross-linked or otherwise manipulated to provide access to the one or more template nucleic acid fragments therein. In some embodiments, prior to partitioning, a tagged deoxyribonucleic acid (DNA) fragment (e.g., transposed genomic DNA sequence) is generated. In some examples, the tagged DNA fragment sequence is flanked on each side by the first tag sequence and a second tag sequence. The one or more template nucleic acid fragments therein may undergo one or more processing operations within the partition. For example, the one or more template nucleic acid fragments may undergo a barcoding process, a ligation process, a reverse transcription process, a template switching process, a linear amplification process, and/or a gap filling process. The resultant processed template nucleic acid fragments may include a barcode sequence (e.g., a nucleic acid barcode sequence, as described herein).

The one or more processed template nucleic acid fragments may be released from the partition (e.g., pooled with contents of other partitions of a plurality of partitions) and may undergo one or more additional processing operations in bulk. For example, sample indexing may occur in bulk solution in which processed template nucleic acid fragments from a plurality of cells, cell beads, and/or cell nuclei from a same sample are in the bulk solution. For example, the one or more processed template nucleic acid fragments may undergo a further barcoding process, a sample identification sequence indexing process, a gap filling process, a dA tailing process, a terminal-transferase process, a phosphorylation process, a ligation process, a nucleic acid amplification process, or a combination thereof. For example, the one or more processed template nucleic acid fragments may be subjected to conditions sufficient to undergo one or more polymerase chain reactions (PCR, such as sequence independent PCR) to generate amplification products corresponding to the one or more processed template nucleic acid fragments. Sequences of such amplification products can be detected using, for example, a nucleic acid sequencing assay and used to identify sequences of the one or more target nucleic acid molecules (e.g., DNA molecules of the cell, cell bead, or cell nucleus from which they derive.

A biological sample (e.g., a nucleic acid sample) may comprise one or more cells, cell beads, and/or cell nuclei. A biological sample may also comprise tissue, which tissue may comprise one or more cells, cell beads, and/or cell nuclei. In some cases, a biological sample may comprise a plurality of cells comprising a plurality of cell nuclei. In some cases, a biological sample may comprise a plurality of cell nuclei, which plurality of cell nuclei are not included within cells (e.g., other components of the cell have degraded, dissociated, dissolved, or otherwise been removed). A biological sample may comprise a plurality of cell-free nucleic acid molecules (e.g., nucleic acid molecules that are not included within cells). For example, a biological sample may comprise a plurality of cell-free fetal DNA (cffDNA) or circulating tumor DNA (ctDNA) or other cell-free nucleic acid molecules (e.g., deriving from degraded cells). Such a biological sample may be processed to separate such cell-free nucleic acid molecules from cells, cell beads, and/or cell nuclei, which cells, cell beads, and/or cell nuclei may be subjected to further processing (e.g., as described herein).

Nucleic acid molecules included within a biological sample may include, for example, DNA molecules and RNA molecules. For example, a biological sample may comprise genomic DNA comprising chromatin (e.g., within a cell, cell bead, or cell nucleus). A biological sample may comprise a plurality of RNA molecules, such as a plurality of pre-mRNA or mRNA molecules (e.g., within a cell, cell bead, or cell nucleus). mRNA molecules and other RNA molecules may comprise a poly A sequence. At least a subset of a plurality of RNA molecules included in a cell or cell bead may be present in a cell nucleus.

A nucleic acid molecule may undergo one or more processing operations within a cell, cell bead, or cell nucleus. For example, chromatin within a cell, cell bead, or cell nucleus may be contacted with a transposase. A transposase may be included within a transposase-nucleic acid complex, which transposase-nucleic acid complex may comprise a transposase molecule and one or more transposon end oligonucleotide molecules. A transposase may be a Tn transposase, such as a Tn3, Tn5, Tn7, Tn10, Tn552, Tn903 transposase. Alternatively, a transposase may be a MuA transposase, a Vibhar transposase (e.g. From Vibrio harveyi), Ac-Ds, Ascot-1, Bs1, Cin4, Copia, En/Spm, F element, hobo, Hsmar1, Hsmar2, IN (HIV), IS1, IS2, IS3, IS4, IS5, IS6, IS10, IS21, IS30, IS50, IS51, IS150, IS256, IS407, IS427, IS630, IS903, IS911, IS982, IS1031, ISL2, L1, Mariner, P element, Tam3, Tcl, Tc3, Tel, THE-1, Tn/O, TnA, Tn3, Tn5, Tn7, Tn10, Tn552, Tn903, Tol1, Tol2, Tn1O, Ty1, any prokaryotic transposase, or any transposase related to and/or derived from those listed above. For example, a transposase may be a Tn5 transposase or a mutated, hyperactive Tn5 transposase. A transposase related to and/or derived from a parent transposase may comprise a peptide fragment with at least about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% amino acid sequence homology to a corresponding peptide fragment of the parent transposase. The peptide fragment may be at least about 10, about 15, about 20, about 25, about 30, about 35, about 40, about 45, about 50, about 60, about 70, about 80, about 90, about 100, about 150, about 200, about 250, about 300, about 400, or about 500 amino acids in length. For example, a transposase derived from Tn5 may comprise a peptide fragment that is 50 amino acids in length and about 80% homologous to a corresponding fragment in a parent Tn5 transposase. Action of a transposase (e.g., insertion) may be facilitated and/or triggered by addition of one or more cations, such as one or more divalent cations (e.g., Ca²⁺, M^{g2+}, or Mn²⁺).

A transposase-nucleic acid complex may comprise one or more nucleic acid molecules. For example, a transposase-nucleic acid complex may comprise one or more transposon end oligonucleotide molecules. A transposon end oligonucleotide molecule may comprise one or more adapter sequences (e.g., comprising one or more primer sequences) and/or one or more transposon end sequences. A transposon end sequence may be, for example, a Tn5 or modified Tn5 transposon end sequence or a Mu transposon end sequence. A transposon end sequence may have a sequence of, for example,

(SEQ ID NO: 1)
AGATGTGTATAAGAGACA.

A primer sequence of a transposon end oligonucleotide molecule may be a sequencing primer, such as an R1 or R2 sequencing primer, or a portion thereof. A sequencing primer may be, for example, a TrueSeq or Nextera sequencing primer. An R1 sequencing primer region may have a sequence of

(SEQ ID NO: 2)
TCTACACTCTTTCCCTACACGACGCTCTTCCGATCT,

or some portion thereof. An R1 sequencing primer region may have a sequence of

(SEQ ID NO: 3)
TCGTCGGCAGCGTCAGATGTGTATAAGAGACAG,

or some portion thereof. A transposon end oligonucleotide molecule may comprise a partial R1 sequence. A partial R1 sequence may be

(SEQ ID NO: 4)
ACTACACGACGCTCTTCCGATCT.

A transposon end oligonucleotide molecule may comprise an R2 sequencing priming region. An R2 sequencing primer region may have a sequence of

(SEQ ID NO: 5)
GTGACTGGAGTTCAGACGTGTGCTCTTCCGATCT,

or some portion thereof. An R2 sequencing primer region may have a sequence of

(SEQ ID NO: 6)
GTCTCGTGGGCTCGGAGATGTGTATAAGAGACAG,

or some portion thereof. A transposon end oligonucleotide molecule may comprise a T7 promoter sequence. A T7 promoter sequence may be

(SEQ ID NO: 7)
TAATACGACTCACTATAG.

A transposon end oligonucleotide molecule may comprise a region at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NO: 1-7. A transposon end oligonucleotide molecule may comprise a P5 sequence and/or a P7 sequence. A transposon end oligonucleotide molecule may comprise a sample index sequence, such as a barcode sequence or unique molecular identifier sequence. One or more transposon end oligonucleotide molecules of a transposase-nucleic acid complex may be attached to a solid support (e.g., a solid or semi-solid particle such as a bead (e.g., gel bead)). A transposon end oligonucleotide molecule may be releasably coupled to a solid support (e.g., a bead). Examples of transposon end oligonucleotide molecules may be found in, for example, PCT Patent Publications Nos. WO2018/218226, WO2014/189957, US. Pat. Pub. 20180340171, and U.S. Pat. No. 10,059,989; each of which are herein incorporated by reference in their entireties.

A primer sequence of a transposon, complement, or portion thereof may be a tag sequence (e.g., a first tag sequence and a second tag sequence). In some instances, a primer sequence of a transposon, complement, or portion thereof may be a sequencing primer. In some cases, a primer sequence of a transposon, complement, or portion thereof may be a sequencing primer or a sequencing primer binding sequence (e.g., for a nucleic acid amplification process). In some cases, a primer sequence of a transposon, complement, or portion thereof may be a sequencing primer or a sequencing primer binding sequence for a PCR process. In some cases, a primer sequence of a transposon, complement, or portion thereof may be a sequencing primer or a sequencing primer binding sequence for a primer extension process. In some cases, an R1 or R2 sequence, complement, or portion thereof may be a sequencing primer or a sequencing primer binding sequence for a nucleic acid amplification process. In some cases, an R1 or R2 sequence, complement, or portion thereof may be a sequencing primer or a sequencing primer binding sequence for a PCR process. In some instances, an R1 or R2 sequence, complement, or portion thereof may be a sequencing primer or a sequencing primer binding sequence for a PCR process. In some cases, an R1 or R2 sequence, complement, or portion thereof may be a sequencing primer or a sequencing primer binding sequence for a primer extension process. In some cases, a primer sequence, a tag sequence, a first primer binding sequence, or a tag binding sequence may be interchangeable. In some embodiments, a tag sequence (e.g., a first tag sequence and a second tag sequence) may comprise a sequencing primer or a sequencing primer binding sequence for a nucleic acid amplification process (e.g., R1 or R2 sequence, complement, or portion thereof).

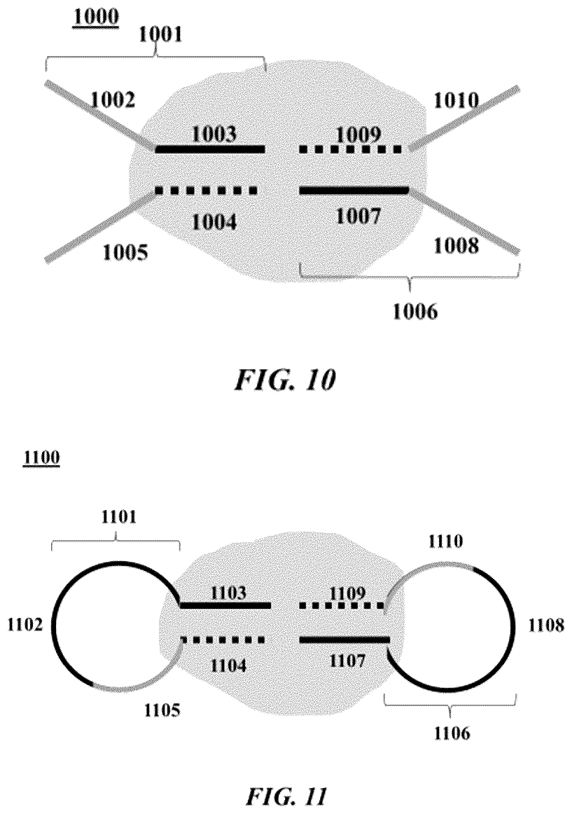

FIG. 9 includes an example of a transposase-nucleic acid complex for use in the methods provided herein. Transposase-nucleic acid complex 900 (e.g., comprising a transpose dimer) comprises partially double-stranded oligonucleotide 901 and partially double-stranded oligonucleotide 905. Partially double-stranded oligonucleotide 901 comprises transposon end sequence 903, first tag sequence 902, and a sequence 904 that is complementary to transposon end sequence 903. Partially double-stranded oligonucleotide 905 comprises transposon end sequence 906, second tag sequence 907, and a sequence 908 that is complementary to transposon end sequence 906. Tag sequences (e.g., sequencing primer sequences) 902 and 907 may be the same or different. In some cases, the first tag sequence (e.g., sequencing primer sequence) 902 may be designated “R1” and the second tag sequence (e.g., sequencing primer sequence) 907 may be designated “R2”. Transposon end sequences 903 and 906 may be the same or different. In some embodiments, transposon end sequences 903 and 906 may alternately be referred to as “mosaic end” or “ME” sequences, while their complementary sequences 904 and 908 may be referred to as “mosaic end reverse complement” or “MErc” sequences.

FIG. 10 includes another example of a transposase-nucleic acid complex for use in the methods provided herein. Transposase-nucleic acid complex 1000 (e.g., comprising a transpose dimer) comprises forked adapters 1001 and 1006, which forked adapters are partially double-stranded oligonucleotides. Partially double-stranded oligonucleotide 1001 comprises transposon end sequence 1003, first tag sequence (e.g., sequencing primer sequence) 1002, second tag sequence (e.g., sequencing primer sequence) 1005, and a sequence 1004 that is complementary to transposon end sequence 1003. Partially double-stranded oligonucleotide 1006 comprises transposon end sequence 1007, first tag sequence (e.g., sequencing primer sequence) 1008, second tag sequence (e.g., sequencing primer sequence) 1010, and a sequence 1009 that is complementary to transposon end sequence 1007. Tag sequences (e.g., sequencing primer sequences) 1002, 1005, 1008, and 1010 may be the same or different. In some cases, tag sequences (e.g., sequencing primer sequences) 1002 and 1008 may be designated “R1” and tag sequences (e.g., sequencing primer sequences) 1005 and 1010 may be designated “R2”. Alternatively, tag sequences (e.g., sequencing primer sequences) 1002 and 1010 may be designated “R1” and tag sequences (e.g., sequencing primer sequences) 1005 and 1008 may be designated “R2”. Alternatively, tag sequences (e.g., sequencing primer sequences) 1002 and 1008 may be designated “R2” and tag sequences (e.g., sequencing primer sequences) 1005 and 1010 may be designated “R1”. Alternatively, tag sequences (e.g., sequencing primer sequences) 1002 and 1010 may be designated “R2” and tag sequences (e.g., sequencing primer sequences) 1005 and 1008 may be designated “R1”. Transposon end sequences 1003 and 1007 may be the same or different. In some embodiments, transposon end sequences may alternately be referred to as “mosaic end” or “ME” sequences, while their complementary sequences 1004 and 1009 may be referred to as “mosaic end reverse complement” or “MErc” sequences.

FIG. 11 shows transposase-nucleic acid complex 1100 (e.g., comprising a transpose dimer) comprising hairpin molecules 1101 and 1106. Hairpin molecule 1101 comprises transposon end sequence 1103, first hairpin sequence 1102, second hairpin sequence 1105, and a sequence 1104 that is complementary to transposon end sequence 1103. Hairpin molecule 1106 comprises transposon end sequence 1107, third hairpin sequence 1108, fourth hairpin sequence 1110, and a sequence 1109 that is complementary to transposon end sequence 1107. Hairpin sequences 1102, 1105, 1108, and 1110 may be the same or different. For example, hairpin sequence 1105 may be the same or different as hairpin sequence 1110, and/or hairpin sequence 1102 may be the same or different as hairpin sequence 1108. Hairpin sequences 1102 and 1108 may be adapter sequences. Hairpin sequences 1105 and 1110 may be a promoter sequence such as T7 recognition or promoter sequences and/or UMI sequences. Transposon end sequences 1103 and 1107 may be the same or different. In some embodiments, transposon end sequences 1103 and 1107 may alternately be referred to as “mosaic end” or “ME” sequences, while their complementary sequences 1104 and 1109 may be referred to as “mosaic end reverse complement” or “Merc” sequences. In some cases, sequence 1104 is a transposon end sequence and 1103 is a sequence complementary to sequence 1104. In some cases, sequence 1109 is a transposon end sequence and 1107 is a sequence complementary to sequence 1109.

Contacting a cell, cell bead, or cell nucleus comprising one or more target nucleic acid molecules (e.g., DNA molecules) with a transposase-nucleic acid complex may generate one or more template nucleic acid fragments that are tagged (e.g., “tagmented fragments” or “tagged fragments”). The one or more template nucleic acid fragments may each comprise a sequence of the one or more target nucleic acid molecules (e.g., a target sequence). The transposase-nucleic acid complex may be configured to target a specific region of the one or more target nucleic acid molecules to provide one or more template nucleic acid fragments comprising specific target sequences. The one or more template nucleic acid fragments may comprise target sequences corresponding to accessible chromatin. Generation of tagmented fragments (e.g., tagged DNA fragments) may take place within a bulk solution. In other cases, generation of tagmented fragments (e.g., tagged DNA fragments) may take place within a partition (e.g., a droplet or well). A template nucleic acid fragment (e.g., tagmented fragment) may comprise one or more gaps (e.g., between a transposon end sequence or complement thereof and a target sequence on one or both strands of a double-stranded fragment). Gaps may be filled via a gap filling process using, e.g., a polymerase (e.g., DNA polymerase), ligase, or reverse transcriptase. In some cases, a mixture of enzymes may be used to repair a partially double-stranded nucleic acid molecule and fill one or more gaps. Gap filling may not include strand displacement. Gaps may be filled within or outside of a partition.

Processing of nucleic acid molecules within a cell, cell bead, or cell nucleus (e.g., generation of template nucleic acid fragments using a transposase-nucleic acid complex and/or generation of additional template nucleic acid fragments using a capture nucleic acid molecule) may occur in a bulk solution comprising a plurality of cells, cell beads, and/or cell nuclei. In some cases, template nucleic acid fragments (e.g., tagmented fragments or tagged DNA fragments) may be generated in bulk solution and/or in a partition.

A plurality of cells, cell beads, and/or cell nuclei (e.g., a plurality of cells, cell beads, and/or cell nuclei that have undergone processing such as a tagmentation process) may be partitioned amongst a plurality of partitions. Partitions may be, for example, droplets or wells. Droplets (e.g., aqueous droplets) may be generated according to the methods provided herein. Partitioning may be performed according to the method provided herein. For example, partitioning a biological particle (e.g., cell, cell bead, or cell nucleus) and one or more reagents may comprise flowing a first phase comprising an aqueous fluid, the biological particle, and the one or more reagents and a second phase comprising a fluid that is immiscible with the aqueous fluid toward a junction. Upon interaction of the first and second phases, a discrete droplet of the first phase comprising the biological particle and the one or more reagents may be formed. The plurality of cells, cell beads, and/or cell nuclei may be partitioned amongst a plurality of partitions such that at least a subset of the plurality of partitions may comprise at most one cell, cell bead, or cell nucleus. Cells, cell beads, and/or cell nuclei may be co-partitioned with one or more reagents such that a partition of at least a subset of the plurality of partitions comprises a single cell, cell bead, or cell nucleus and one or more reagents. The one or more reagents may include, for example, enzymes (e.g., polymerases, reverse transcriptases, ligases, etc.), nucleic acid barcode molecules (e.g., nucleic acid barcode molecules comprising one or more barcode sequences, such as nucleic acid barcode molecules coupled to one or more beads), template switching oligonucleotides, deoxynucleotide triphosphates, buffers, lysis agents, primers, barcodes, detergents, reducing agents, chelating agents, oxidizing agents, nanoparticles, beads, antibodies, or any other useful reagents. Enzymes may include, for example, temperature-sensitive enzymes, pH-sensitive enzymes, light-sensitive enzymes, reverse transcriptases, proteases, ligases, polymerases, kinases, restriction enzymes, nucleases, protease inhibitors, exonucleases, and nuclease inhibitors.

A reagent of the one or more reagents may be useful for lysing or permeabilizing a cell, cell bead, or cell nucleus, or otherwise providing access to nucleic acid molecules and/or template nucleic acid fragments therein. A cell may be lysed using a lysis agent such as a bioactive agent. A bioactive agent useful for lysing a cell may be, for example, an enzyme (e.g., as described herein). An enzyme used to lyse a cell may or may not be capable of carrying out additional actions such as degrading one or more RNA molecules. Alternatively, an ionic, zwitterionic, or non-ionic surfactant may be used to lyse a cell. Examples of surfactants include, but are not limited to, TritonX-100, Tween 20, sarcosyl, or sodium dodecyl sulfate. Cell lysis may also be achieved using a cellular disruption method such as an electroporation or a thermal, acoustic, or mechanical disruption method. Alternatively, a cell may be permeabilized to provide access to a plurality of nucleic acid molecules included therein. Permeabilization may involve partially or completely dissolving or disrupting a cell membrane or a portion thereof. Permeabilization may be achieved by, for example, contacting a cell membrane with an organic solvent or a detergent such as Triton X-100 or NP-40. By lysing or permeabilizing a cell, cell bead, or cell nucleus within a partition (e.g., droplet) to provide access to the plurality of nucleic acid molecules and/or template nucleic acid fragments therein, molecules originating from the same cell, cell bead, or cell nucleus may be isolated within the same partition.

A partition of a plurality of partitions (e.g., a partition comprising a cell, cell bead, and/or cell nucleus) may comprise one or more beads (e.g., gel beads). A bead may be a gel bead. A bead may comprise a plurality of nucleic acid barcode molecules (e.g., nucleic acid molecules each comprising one or more barcode sequences, as described herein). A bead may comprise at least 10,000 nucleic acid barcode molecules attached thereto. For example, the bead may comprise at least 100,000, 1,000,000, or 10,000,000 nucleic acid barcode molecules attached thereto. The plurality of nucleic acid barcode molecules may be releasably attached to the bead. The plurality of nucleic acid barcode molecules may be releasable from the bead upon application of a stimulus. Such a stimulus may be selected from the group consisting of a thermal stimulus, a photo stimulus, and a chemical stimulus. For example, the stimulus may be a reducing agent such as dithiothreitol Application of a stimulus may result in one or more of (i) cleavage of a linkage between nucleic acid barcode molecules of the plurality of nucleic acid barcode molecules and the bead, and (ii) degradation or dissolution of the bead to release nucleic acid barcode molecules of the plurality of nucleic acid barcode molecules from the bead.

In an aspect, the present disclosure provides a method for barcoding a nucleic acid molecule. A nucleic acid barcode sequence may be attached, associated, or linked to a nucleic acid molecule before a nucleic acid sample identification sequence is attached, associated, or linked to the nucleic acid molecule. A nucleic acid barcode sequence may also be attached, associated, or linked to a nucleic acid molecule after a nucleic acid sample identification sequence is attached, associated, or linked to the nucleic acid molecule. In other cases, a nucleic acid barcode sequence may be attached, associated, or linked to a nucleic acid molecule at the same time when a nucleic acid sample identification sequence is attached, associated, or linked to the nucleic acid molecule. In some cases, a first nucleic acid sample identification sequence may be attached, associated, or linked to a nucleic acid molecule at the same time when a second nucleic acid sample identification sequence is attached, associated, or linked to the nucleic acid molecule. A first nucleic acid sample identification sequence may also be attached, associated, or linked to a nucleic acid molecule before a second nucleic acid sample identification sequence is attached, associated, or linked to the nucleic acid molecule. In some cases, a nucleic acid sample identification sequence may also be attached, associated, or linked to a nucleic acid molecule after a second nucleic acid sample identification sequence is attached, associated, or linked to the nucleic acid molecule.

A plurality of nucleic acid barcode molecules attached (e.g., releasably attached) to a bead (e.g., gel bead) may be suitable for barcoding template nucleic acid fragments or additional template nucleic acid fragments deriving from DNA molecules of the plurality of cells, cell beads, and/or cell nuclei. For example, a nucleic acid barcode molecule of a plurality of nucleic acid barcode molecule may comprise a barcode sequence, unique molecular identifier (UMI) sequence, tag binding sequence, sequencing primer sequence, universal primer sequence, sequencing adapter or primer, flow cell adapter sequence, or any other useful feature. In an example, a nucleic acid barcode molecule of a plurality of nucleic acid barcode molecules attached to a bead may comprise a spacer sequence (e.g., a first primer binding sequence), a barcode sequence, a capture sequence, a tag binding sequence, and/or a sequencing primer sequence (e.g., tag binding sequence) or portion thereof (e.g., an R1 or R2 sequence or portion thereof), or a complement of any of these sequences. These sequences may be arranged in any useful order and may be linked or may include one or more spacer sequences disposed between them. In some instances, the spacer sequence is a first primer binding sequence that is complementary to a primer sequence of a primer molecule comprising a sample identification sequence for adding a sample identification (e.g., index) sequence to a barcoded tagged deoxyribonucleic acid (DNA) fragment sequence. For instance, the sequencing primer (e.g., tag binding sequence) or portion thereof may be disposed at an end of the nucleic acid barcode molecule that is farthest from (e.g., distal to) the bead (e.g., most available to template nucleic acid fragments for interaction). In another example, a nucleic acid barcode molecule of a plurality of nucleic acid barcode molecules attached to a bead may comprise a spacer sequence (e.g., first primer binding sequence), a barcode sequence, a sequencing primer sequence (e.g., tag sequence) or portion thereof (e.g., an R1 or R2 sequence or portion thereof), and a UMI sequence, or a complement of any of these sequences. In another example, a nucleic acid barcode molecule of a plurality of nucleic acid barcode molecules attached to a bead may comprise a spacer sequence (e.g., first primer binding sequence), a barcode sequence, a sequencing primer sequence (e.g., tag binding sequence) or portion thereof (e.g., an R1 or R2 sequence or portion thereof), or a complement of any of these sequences. In one example, a nucleic acid barcode molecule of a plurality of nucleic acid barcode molecules attached to a bead may comprise, from 5′ to 3′ end, a spacer sequence, a barcode sequence, a sequencing primer sequence or portion thereof (R1 sequence or portion thereof), or a complement of any of these sequences. In some cases, a nucleic acid barcode molecule comprises, from 5′ to 3′: a first primer binding sequence, a barcode sequence, and a first tag binding sequence complementary to the first tag sequence. In some cases, the nucleic acid barcode molecule does not have a sequencer specific flow cell attachment sequence (e.g., a P5, P7, or partial P5 or P7 sequence). In some cases, the first primer binding sequence of a nucleic acid barcode molecule is not a sequencer specific flow cell attachment sequence (e.g., a P5, P7, or partial P5 or P7 sequence). In some cases, the first primer binding sequence of a nucleic acid barcode molecule (e.g., at the 5′ end of the nucleic acid barcode molecule) is not a sequencing primer sequence (e.g., tag sequence) or portion thereof (e.g., an R1 or R2 sequence or portion thereof). In some cases, the first primer binding sequence of a nucleic acid barcode molecule is not a sequencer specific flow cell attachment sequence (e.g., a P5, P7, or partial P5 or P7 sequence) or a sequencing primer sequence (e.g., tag sequence) or portion thereof (e.g., an R1 or R2 sequence or portion thereof).

In some other cases, a nucleic acid barcode molecule may further comprise a capture sequence, which capture sequence may be a targeted capture sequence or comprise a template switch sequence (e.g., comprising a polyC or poly G sequence). These sequences may be arranged in any useful order and may be linked or may include one or more spacer sequences disposed between them. For instance, the flow cell adapter sequence may be disposed near (e.g., proximal to) an end of the nucleic acid barcode molecule that is closest to the bead, while the capture sequence or template switch sequence may be disposed at an end of the nucleic acid barcode molecule that is furthest from the bead (e.g., most available to template nucleic acid fragments for interaction).

A spacer sequence can comprise about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more nucleotides within the sequence of the nucleic acid molecules (e.g., oligonucleotides). A spacer sequence can comprise can comprise from 1 to 10, from 2 to 11, from 3 to 12, from 4 to 13, from 5 to 14, from 6 to 15, from 7 to 16, from 8 to 17, from 9 to 18, from 10 to 19, from 11 to 20, from 12 to 21, from 13 to 22, from 14 to 23, from 15 to 24, from 16 to 25, from 17 to 26, from 18 to 27, from 19 to 28, from 20 to 29, from 21 to 30, from 22 to 31, from 23 to 32, from 24 to 33, from 25 to 34, from 26 to 35, from 27 to 36, from 28 to 37, from 29 to 38, from 30 to 39, from 31 to 40, from 32 to 41, from 33 to 42, from 34 to 43, from 35 to 44, from 36 to 45, from 37 to 46, from 38 to 47, from 39 to 48, from 40 to 49, from 41 to 50, from 42 to 51, from 43 to 52, from 44 to 53, from 45 to 54, from 46 to 55, from 47 to 56, from 48 to 57, from 49 to 58, from 50 to 59, from 51 to 60, from 52 to 61, from 53 to 62, from 54 to 63, from 55 to 64, from 56 to 65, from 57 to 66, from 58 to 67, from 59 to 68, from 60 to 69, from 61 to 70, from 62 to 71, from 63 to 72, from 64 to 73, from 65 to 74, from 66 to 75, from 67 to 76, from 68 to 77, from 69 to 78, from 70 to 79, from 71 to 80, from 72 to 81, from 73 to 82, from 74 to 83, from 75 to 84, from 76 to 85, from 77 to 86, from 78 to 87, from 79 to 88, from 80 to 89, from 81 to 90, from 82 to 91, from 83 to 92, from 84 to 93, from 85 to 94, from 86 to 95, from 87 to 96, from 88 to 97, from 89 to 98, from 90 to 99, or from 91 to 100 nucleotides. In some cases, a spacer sequence may be about 6, 7, 8, 9, 10, 11, 12, 13, 14, 15, 16, 17, 18, 19, 20 nucleotides or longer. In some cases, the length of a spacer sequence may be at least about 6, 7, 8, 9, 10, 11, 12, 13, 14, 15, 16, 17, 18, 19, 20 nucleotides or longer. In some cases, the length of a spacer sequence may be at most about 6, 7, 8, 9, 10, 11, 12, 13, 14, 15, 16, 17, 18, 19, 20 nucleotides or shorter. In some cases, the length of a spacer sequence may be about 16 nucleotides.

A spacer sequence, complement, or portion thereof may be a primer or a primer binding sequence for a nucleic acid amplification process. In some cases, a spacer sequence, complement, or portion thereof may be a primer or a primer binding sequence for a PCR process. In some cases, a spacer sequence, complement, or portion thereof may be a first primer sequence; and a primer sequence of a transposon, complement, or portion thereof may be a second primer sequence for a nucleic acid amplification process. In some instances, a spacer sequence, complement, or portion thereof may be a first primer sequence; and a primer sequence of a transposon, complement, or portion thereof may be a second primer sequence for a PCR process. In some cases, a spacer sequence, complement, or portion thereof may be a first primer sequence; and an R2 sequence, complement, or portion thereof may be a second primer sequence for a nucleic acid amplification process. In some instances, a spacer sequence, complement, or portion thereof may be a first primer sequence; and an R2 sequence, complement, or portion thereof may be a second primer sequence for a PCR process.

In some cases, a spacer sequence, complement, or portion thereof may be a first ligation indexing adaptor sequence on one end of a nucleic acid molecule; and a primer sequence of a transposon, complement, or portion thereof may be a second ligation indexing adaptor sequence on another end of the nucleic acid molecule for a ligation process. In some instances, a spacer sequence, complement, or portion thereof may be a first ligation indexing adaptor sequence on a 5′ end of a nucleic acid molecule; and a primer sequence of a transposon, complement, or portion thereof may be a second ligation indexing adaptor sequence on a 3′ end of the nucleic acid molecule for a ligation process. In some cases, a spacer sequence, complement, or portion thereof may be a first ligation indexing adaptor sequence on one end of a nucleic acid molecule; and an R2 sequence, complement, or portion thereof may be a second ligation indexing adaptor sequence on another end of the nucleic acid molecule for a ligation process. In some instances, a spacer sequence, complement, or portion thereof may be a first ligation indexing adaptor sequence on a 5′ end of a nucleic acid molecule; and an R2 sequence, complement, or portion thereof may be a second ligation indexing adaptor sequence on a 3′ end of the nucleic acid molecule for a ligation process.

All of the nucleic acid barcode molecules attached (e.g., releasably attached) to a bead (e.g., gel bead) of a plurality of beads may be the same. For example, all of the nucleic acid barcode molecules attached to the bead may have the same nucleic acid sequence. In such an instance, all of the nucleic acid barcode molecules attached to the bead may comprise the same flow cell adapter sequence, sequencing primer or portion thereof, and barcode sequence. The barcode sequence of a plurality of nucleic acid barcode molecules attached to a bead of a plurality of beads may be different from other barcode sequences of other nucleic acid barcode molecules attached to other beads of the plurality of beads. For example, a plurality of beads may comprise a plurality of barcode sequences, such that, for at least a subset of the plurality of beads, each bead comprises a different barcode sequence of the plurality of barcode sequences. This differentiation may permit template nucleic acid fragments (e.g., included within cells, cell beads, and/or cell nuclei) co-partitioned with a plurality of beads between a plurality of partitions to be differentially barcoded within their respective partitions, such that the template nucleic acid fragments or molecules derived therefrom may be identified with the partition (and thus the cell, cell bead, and/or cell nucleus) to which they correspond (e.g., using a nucleic acid sequencing assay, as described herein). A barcode sequence may comprise between 4-20 nucleotides. A barcode sequence may comprise one or more segments, which segments may range in size from 2-20 nucleotides, such as from 4-20 nucleotides. Such segments may be combined to form barcode sequences using a combinatorial assembly method, such as a split-pool method. Details of such methods can be found, for example, in PCT/US2018/061391, filed Nov. 15, 2018, and 20190249226, each of which are herein incorporated by reference in their entireties.

In some cases, nucleic acid barcode molecules attached to a bead may not be the same. For example, the plurality of nucleic acid barcode molecules attached to a bead may each comprise a UMI sequence, which UMI sequence varies across the plurality of nucleic acid barcode molecules. All other sequences of the plurality of nucleic acid barcode molecules attached to the bead may be the same.

In some cases, a bead may comprise multiple different nucleic acid barcode molecules attached thereto. For example, a bead may comprise a first plurality of nucleic acid barcode molecules and a second plurality of nucleic acid barcode molecules, which first plurality of nucleic acid barcode molecules is different than the second plurality of nucleic acid barcode molecules. The first plurality of nucleic acid barcode molecules and the second plurality of nucleic acid barcode molecules coupled to a bead may comprise one or more shared sequences. For example, each nucleic acid barcode molecule of the first plurality of nucleic acid barcode molecules and each nucleic acid barcode molecule of the second plurality of nucleic acid barcode molecules may comprise the same barcode sequence (e.g., as described herein). Such a barcode sequence may be prepared using a combinatorial assembly process (e.g., as described herein). For example, barcode sequences may comprise identical barcode sequence segments. Similarly, each nucleic acid barcode molecule of the first plurality of nucleic acid barcode molecules coupled to a bead may comprise the same flow cell adapter sequence and/or sequencing primer or portion thereof as each nucleic acid barcode molecule of the second plurality of nucleic acid barcode molecules coupled to the bead. In an example, each nucleic acid barcode molecule of the first plurality of nucleic acid barcode molecules coupled to a bead comprises a sequencing primer, and each nucleic acid barcode molecule of the second plurality of nucleic acid barcode molecules coupled to the bead comprises a portion of the same sequencing primer. In some instances, each nucleic acid barcode molecule of the first plurality of nucleic acid barcode molecules coupled to a bead may comprise a first sequencing primer (e.g., a TruSeq R1 sequence) a barcode sequence, and a first functional sequence, and each nucleic acid barcode molecule of the second plurality of nucleic acid barcode molecules coupled to the bead may comprise a second sequencing primer (e.g., a Nextera R1 sequence, or a portion thereof), the barcode sequence, and a second functional sequence. Sequences shared between different sets of nucleic acid barcode molecules coupled to the same bead may be included in the same or different order and may be separated by the same or different sequences. Alternatively or in addition, the first plurality of nucleic acid barcode molecules and the second plurality of nucleic acid barcode molecules coupled to a bead may include one or more different sequences. For example, each nucleic acid barcode molecule of a first plurality of nucleic acid barcode molecules coupled to a bead of a plurality of beads may comprise one or more of a flow cell adapter sequence, a barcode sequence, UMI sequence, capture sequence, and a sequencing primer or portion thereof, while each nucleic acid barcode molecule of a second plurality of nucleic acid barcode molecules coupled to the bead may comprise one or more of a flow cell adapter sequence (e.g., the same flow cell adapter sequence), a barcode sequence (e.g., the same barcode sequence), UMI sequence, capture sequence, and a sequencing primer or portion thereof (e.g., the same sequencing primer or portion thereof). Nucleic acid barcode molecules of the first plurality of nucleic acid barcode molecules may not include a UMI sequence or capture sequence. A bead comprising multiple different populations of nucleic acid barcode molecules, such as a first plurality of nucleic acid molecules and a second plurality of nucleic acid molecules (e.g., as described above), may be referred to as a “multi-functional bead.”

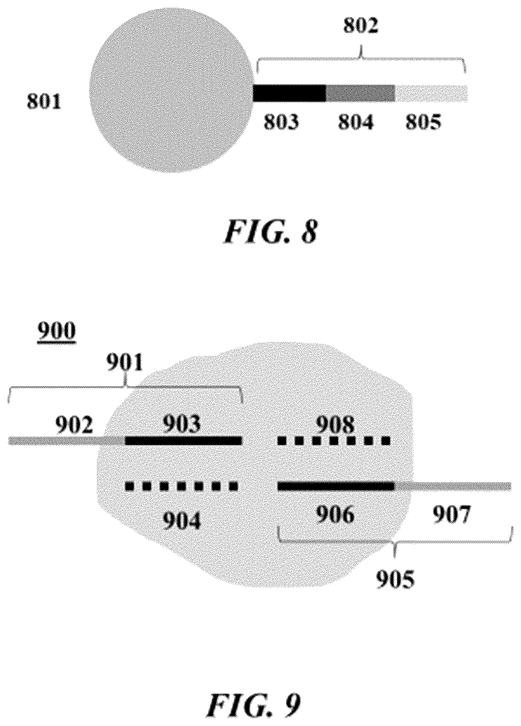

FIG. 8 shows an example of a bead for use according to the method provided herein. A bead 801 may be partitioned with a cell, cell bead, or cell nucleus into a partition of a plurality of partitions (e.g., droplets or wells). Bead 801 may comprise a nucleic acid barcode molecule 802. Nucleic acid barcode molecule 802 may comprise sequences 803, 804, and 805. Sequence 803 may be, for example, a spacer sequence (e.g., a first primer binding sequence). Sequence 804 may be, for example, a barcode sequence. Sequence 805 may be, for example, a tag binding sequence (e.g., a sequencing primer sequence) or portion thereof (e.g., an R1 or R2 primer sequence, or portion thereof). Nucleic acid barcode molecule 802 may also include additional sequences, such as a UMI sequence. Bead 801 may comprise a plurality of nucleic acid barcode molecules. The plurality of nucleic acid barcode molecules may each comprise a common barcode sequence, such as sequence 804 or portion thereof.

In an example, a cell, cell bead, or cell nucleus comprising chromatin is provided. The chromatin in the cell, cell bead, or cell nucleus may be processed to provide a template nucleic acid fragment derived from the chromatin (e.g., a tagmented fragment or tagged DNA fragment, as described herein). The chromatin may be processed in bulk solution and/or in partition. The configuration of the template nucleic acid fragment may be at least partially dependent on the structure of the transposase-nucleic acid complex used to generate the template nucleic acid fragment. For example, a transposase-nucleic acid complex such as that shown in FIG. 9 may be used to prepare the template nucleic acid fragment. The template nucleic acid fragment may be at least partially double-stranded. The template nucleic acid fragment may comprise a double-stranded region comprising sequences of chromatin of the cell, cell bead, or cell nucleus. A first end of a first strand of the double-stranded region may be linked to a first transposon end sequence (e.g., mosaic end sequence), which first transposon end sequence may be linked to a first sequencing primer or portion thereof. A first end of the second strand of the double-stranded region, which end is opposite the first end of the first strand, may be linked to a second transposon end sequence (e.g., mosaic end sequence), which second transposon end sequence may be linked to a second sequencing primer or portion thereof. The second transposon end sequence may be the same as or different from the first transposon end sequence. The first sequencing primer or portion thereof may be the same as or different from the second sequencing primer or portion thereof. In some cases, the first tag sequence (e.g., first sequencing primer) or portion thereof may be an R1 sequence or portion thereof, and the second tag sequence (e.g., second sequencing primer) or portion thereof may be an R2 sequence or portion thereof. The first transposon end sequence may be hybridized to a first complementary sequence (e.g., mosaic end reverse complement sequence), which first complementary sequence may not be linked to a second end of the second strand of the double-stranded region of the first template nucleic acid fragment. Similarly, the second transposon end sequence may be hybridized to a second complementary sequence (e.g., mosaic end reverse complement sequence), which second complementary sequence may not be linked to a second end of the first strand of the double-stranded region of the first template nucleic acid fragment. In other words, the template nucleic acid fragment may comprise one or more gaps. In some cases, the one or more gaps may be approximately 9 bp in length each.

The cell, cell bead, or cell nucleus comprising a template nucleic acid fragment (e.g., tagmented fragment or tagged DNA fragment) may be co-partitioned with one or more reagents into a partition of a plurality of partitions (e.g., as described herein). The template nucleic acid fragment (e.g., tagmented fragment or tagged DNA fragment) may be generated prior to being co-partitioned with one or more reagents into a partition. The partition may be, for example, a droplet or well. The partition may comprise one or more beads (e.g., as described herein). A bead of the one or more beads may comprise a first plurality of nucleic acid barcode molecules. A nucleic acid barcode molecule of the first plurality of nucleic acid barcode molecules may comprise one or more of a spacer sequence (e.g., first primer binding sequence), a barcode sequence, and a tag binding sequence (e.g., sequencing primer) or portion thereof (e.g., an R1 or R2 sequence or portion thereof, or a complement thereof). The sequencing primer or portion thereof may be complementary to a sequence of the first template nucleic acid fragment.

Within the partition, the cell, cell bead, or cell nucleus may be lysed or permeabilized to provide access to the template nucleic acid fragments therein (e.g., as described herein). The second template nucleic acid fragment may be generated after the cell, cell bead, or cell nucleus is lysed or permeabilized.

The first and second template nucleic acid fragments may undergo processing within the partition. Within the partition, the gaps in the template nucleic acid molecule may be filled via a gap filling extension process (e.g., using a DNA polymerase or reverse transcriptase). The resultant double-stranded nucleic acid molecule may be denatured to provide a single strand comprising a chromatin sequence flanked by transposon end sequences and/or sequences complementary to transposon end sequences. Each transposon end sequence and/or sequence complementary to transposon end sequence may be linked to a sequencing primer or portion thereof, or a complement thereof (e.g., an R1 or R2 sequence or a portion thereof, or a complement thereof). A nucleic acid barcode molecule of the first plurality of nucleic acid barcode molecules may hybridize to a sequencing primer or portion thereof, or a complement thereof, of the single strand. A primer extension reaction may then be used to generate a complement of the single strand (e.g., using a DNA polymerase or reverse transcriptase). Such a process may amount to a linear amplification process. This process incorporates the barcode sequence of the nucleic acid barcode molecule of the first plurality of nucleic acid barcode molecules, or a complement thereof. The resultant double-stranded molecule may be denatured to provide a single strand comprising the spacer sequence, or complement thereof, of the nucleic acid barcode molecule of the first plurality of nucleic acid barcode molecules; barcode sequence, or complement thereof, of the nucleic acid barcode molecule of the first plurality of nucleic acid barcode molecules; sequencing primer or portion thereof, or complement thereof, of the nucleic acid barcode molecule of the first plurality of nucleic acid barcode molecules; transposon end sequences, and/or complements thereof; second sequencing primer or portion thereof, or complement thereof. An additional amplification process may or may not be performed within a partition. For example, exponential amplification may or may not be performed within a partition.

A nucleic acid barcode sequence may be attached, associated, or linked to a nucleic acid molecule by a process. A process can comprise a phosphorylation process, ligation process, nucleic acid amplification process, primer extension process, nucleic acid hybridization, or a combination thereof. A nucleic acid amplification process can comprise primer extension, loop-mediated isothermal amplification (LAMP), nucleic acid sequence-based amplification (NASBA), strand displacement amplification (SDA), helicase dependent amplification (HAD), transcription-mediated amplification (TMA), multiple cross displacement amplification (MCDA), polymerase chain reaction (PCR), any derivatives herein and thereof, or combinations herein and thereof. A nucleic acid amplification process can comprise an enzyme. An enzyme can comprise a DNA polymerase, DNA helicase, reverse transcriptase, RNA polymerase, recombinase, restriction endonuclease, any derivatives herein and thereof, or any combinations herein and thereof. A nucleic acid barcode may be attached, associated, or linked to a nucleic acid molecule by a linear amplification process. A linear amplification process can comprise a primer extension process.

The linear amplification products corresponding to the chromatin of the cell, cell bead, or cell nucleus included within the partition of the plurality of partitions may be recovered from the partition. For example, the contents of the plurality of partitions may be pooled to provide the linear amplification products in a bulk solution. The linear amplification product corresponding to the chromatin may then be subjected to conditions sufficient to undergo one or more nucleic acid amplification reactions (e.g., PCR) to generate one or more amplification products corresponding to the chromatin.

Provided here are also methods for dual indexing (e.g., attaching a pair of sample identification sequences to) a nucleic acid molecule. A nucleic acid sample identification sequence may be attached, associated, or linked to a nucleic acid molecule by a process. The methods can comprise a phosphorylation process, ligation process, nucleic acid amplification process, primer extension process, nucleic acid hybridization, or a combination thereof. A nucleic acid amplification process can comprise primer extension, loop-mediated isothermal amplification (LAMP), nucleic acid sequence-based amplification (NASBA), strand displacement amplification (SDA), helicase dependent amplification (HAD), transcription-mediated amplification (TMA), multiple cross displacement amplification (MCDA), polymerase chain reaction (PCR), any derivatives herein and thereof, or combinations herein and thereof. A nucleic acid amplification process can comprise an enzyme. An enzyme can comprise a DNA polymerase, DNA helicase, reverse transcriptase, RNA polymerase, recombinase, restriction endonuclease, any derivatives herein and thereof, or any combinations herein and thereof.

In some cases, a nucleic acid amplification process can comprise a linear amplification of a template nucleic acid. In other cases, a nucleic acid amplification process can comprise a non-linear amplification of a template nucleic acid. A non-linear amplification can comprise an exponential or logarithmic amplification of a template nucleic acid. In some cases, a nucleic acid can comprise a linear and non-linear amplification of a template nucleic acid.

In some cases, a primer used to amplify a nucleic acid molecule may comprise a sample identification sequence, a flow cell adaptor molecule, a spacer sequence, or any complements thereof or combinations thereof. In some instances, a first primer used to amplify a nucleic acid molecule may comprise a first sample identification sequence (e.g., an i5 index sequence), a first flow cell adaptor molecule (e.g., a P5 sequence), a spacer sequence, any complements thereof or combinations thereof. A second primer used to amplify the nucleic acid molecule may comprise a second sample identification sequence (e.g., an i7 index sequence) and a second flow cell adaptor molecule (e.g., a P7 sequence). In some cases, a primer may comprise, from 5′ to 3′, a flow cell adaptor sequence or any complements thereof (e.g., a P5 or P7 sequence), a sample identification sequence or any complements thereof (e.g., an i5 or i7 index sequence), a primer sequence or complement thereof (e.g., a spacer sequence), or a tag sequence (e.g., an R2 sequence). In one example, a first primer may comprise, from 5′ to 3′, a flow cell adaptor sequence or any complements thereof (e.g., a P5 sequence), a sample identification sequence or any complements thereof (e.g., an i5 index sequence), a primer sequence or complement thereof (e.g., a space sequence). In another example, a second primer may comprise, from 5′ to 3′, a flow cell adaptor sequence or any complements thereof (e.g., a P7 sequence), a sample identification sequence or any complements thereof (e.g., an i7 index sequence), a tag sequence or complement thereof (e.g., an R2 sequence). These nucleotides may be completely contiguous, e.g., in a single stretch of adjacent nucleotides, or they may be separated into two or more separate subsequences that are separated by 1 or more nucleotides. In some cases, a primer sequence of the primer molecule does not hybridize to a tag sequence or complement thereof (e.g., a sequencing primer) or portion thereof (e.g., an R1 sequence or portion thereof, or a complement thereof).

In some cases, a first and second primer sequence can be used in a PCR amplification process to amplify a template nucleic acid molecule. In other cases, a primer may be used in a primer extension process to extend a template nucleic acid molecule.

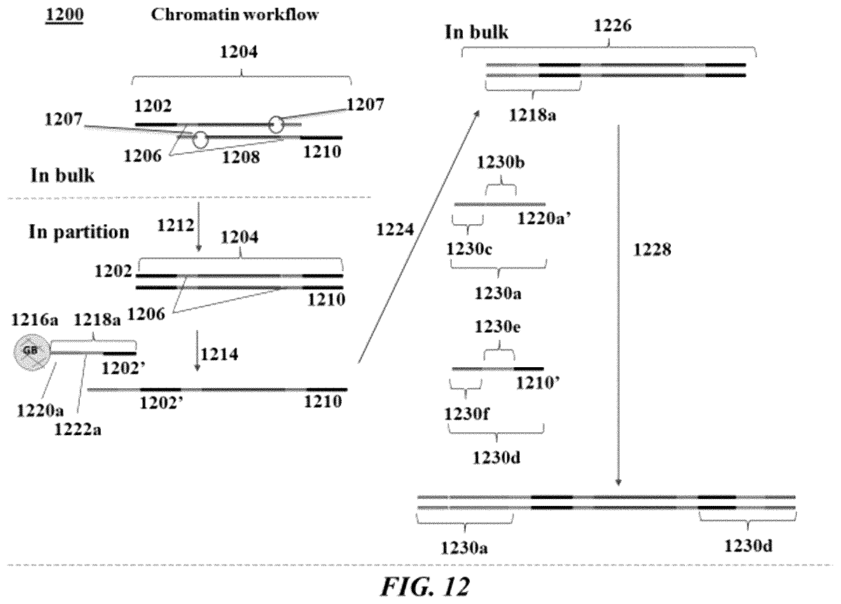

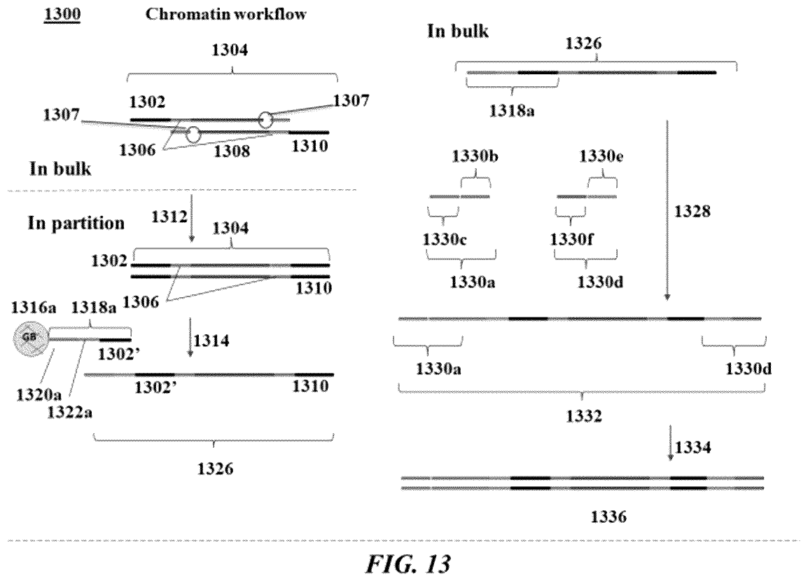

FIG. 12 shows an example schematic corresponding to the preceding example. Panel 1200 shows a workflow corresponding to processing of chromatin from a cell, cell bead, or cell nucleus.

As shown in panel 1200, in bulk solution, chromatin included within a cell, cell bead, or cell nucleus is processed (e.g., as described herein) to provide a template nucleic acid fragment (e.g., tagmented fragment or tagged DNA fragment) 1204 comprising insert sequence 1208 (e.g., region of open chromatin) and a complement thereof, transposon end sequences 1206 and complements thereof, first tag sequence (e.g., sequencing primer) or portion thereof 1202 (e.g., an R1 sequence), second tag sequence (e.g., sequencing primer) or portion thereof 1210 (e.g., an R2 sequence), and gaps 1207. Template nucleic acid fragment 1204 may then be partitioned within a partition (e.g., a droplet or well, as described herein). Within the partition, the cell, cell bead, or cell nucleus comprising template nucleic acid fragment 1204 may be lysed, permeabilized, or otherwise processed to provide access to template nucleic acid fragment 1204 (and one or more RNA molecules) therein. Gaps 1207 may be filled 1212 via a gap filling extension process (e.g., using a DNA polymerase). The partition may include a bead (e.g., gel bead) 1216a coupled to a nucleic acid barcode molecule 1218a. Nucleic acid barcode molecule 1218a may comprise a spacer sequence (e.g., first primer binding sequence) 1220a, a barcode sequence 1222a, and a tag binding sequence (e.g., sequencing primer) or portion thereof or complement thereof 1202′. Sequence 1202′ may hybridize to sequence 1202 of template nucleic acid fragment 1204, or its complement, and undergo primer extension 1214 to yield a strand comprising sequences 1220a, 1222a, 1202′, 1210, and insert sequence 1208 or a complement thereof. The contents of the partition may then be recovered in bulk solution (e.g., a droplet may be broken) to provide the strand in bulk solution. This strand may undergo amplification (e.g., PCR) 1224 to provide a double-stranded sequence 1226. Sequence 1226 is then dual-indexed with a PCR amplification 1228 using a pair of primer molecules 1230a and 1230d. 1230a may comprise a primer sequence 1220a′ that can hybridize to the spacer (e.g., first primer binding sequence) or portion thereof or complement thereof 1220a, a first sample identification sequence 1230b (e.g., an i5 index sequence), and a first flow cell adapter sequence 1230c (e.g., a P5 sequence). 1230d may comprise a sequence 1210′ that can hybridize to the second tag sequence (e.g., sequencing primer) or portion thereof or complement thereof 1210, a second sample identification sequence 1230e (e.g., an i7 index sequence), and a second flow cell adapter sequence 1230e (e.g., a P7 sequence). The amplification product of 1228 may include sequences of the nucleic acid barcode molecule 1218a, the original chromatin molecule, the first and second sample identification sequence 1230b and 1230e, and the first and second flow cell adapter sequence 1230c and 1230f.

In some of any such cases, the nucleic acid molecules described here may include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a tag sequence, a first primer or primer binding sequence (a spacer sequence), a sequencing primer or primer binding sequence (such as an R1, R2, or partial R1 or R2 sequence). In some cases, such functional sequences or domains can be selected for compatibility with a variety of different sequencing systems, e.g., 454 Sequencing, Ion Torrent Proton or PGM, Illumina X10, etc., or other platforms from Illumina, BGI, Qiagen, Thermo-Fisher, PacBio, and Roche, and the requirements thereof.

In some cases, a nucleic acid molecule may be indexed or assigned a sample identification sequence (or a pair of sample identification sequences) by a ligation adaptor. In some instances, a nucleic acid molecule may be indexed or assigned a sample identification sequence before a non-linear amplification of the nucleic acid molecule. In some cases, a nucleic acid molecule may be indexed or assigned a sample identification sequence after a linear amplification of the nucleic acid molecule. In some cases, a nucleic acid molecule may undergo a non-linear amplification process after being ligated to a ligation adaptor comprising a sample identification sequence. In some cases, the 5′ and 3′ end of a nucleic acid molecule may undergo modification. In some cases, a 5′ or 3′ end modification may comprise a process that phosphorylates or dephosphorylates the 5′ or 3′ end of a nucleic acid molecule. In some cases, a 5′ or 3′ end modification may comprise a process that creates a blunt end or a staggered end at the 5′ or 3′ end of a nucleic acid molecule. In some cases, a 5′ or 3′ end modification may comprise a process that creates a 5′ or 3′ nucleotide overhang for a nucleic acid molecule. In some instances, a 5′ or 3′ end modification of a nucleic acid molecule may facilitate the ligation of a ligation adaptor to the 5′ or 3′ end of the nucleic acid molecule.

In some cases, an adaptor may be ligated to a nucleic acid molecule. An adaptor may comprise a sample identification sequence and a flow cell adaptor molecule. In some instances, a first adaptor may comprise a sample identification sequence (e.g., an i5 index sequence) and a first flow cell adaptor molecule (e.g., a P5 sequence), and a second adaptor may comprise a second sample identification sequence (e.g., an i7 index sequence) and a second flow cell adaptor molecule (e.g., a P7 sequence). In some cases, a first and second adaptor can be used in a ligation process.

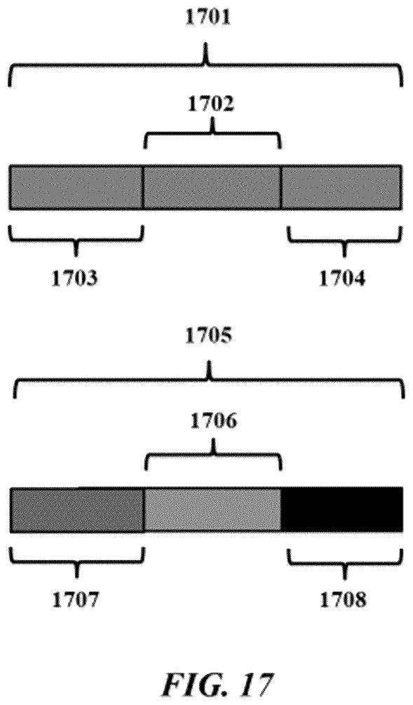

FIG. 17 shows two examples of primers for dual indexing nucleic acids molecules. Primer 1701 comprises, from 5′ to 3′, a first flow cell adaptor sequence 1703 (e.g., a P5 sequence or its sequence complement), a first sample identification sequence 1702 (e.g., an i5 index sequence or its sequence complement), and a first primer sequence 1704 (e.g., a spacer sequence or its sequence complement). Primer 1705 comprises, from 5′ to 3′, a second flow cell adaptor sequence complement 1707 (e.g., a P7 sequence or its sequence complement), a second sample identification sequence complement 1706 (e.g., an i7 index sequence or its sequence complement), and a second sequencing primer/tag sequence complement 1708 (e.g., an R2 sequence or its sequence complement).

In some cases, a plurality of first primers can comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, or more individually unique or distinct first sample identification sequences. In some instances, a plurality of first primers can also comprise from 1 to 10, from 5 to 15, from 10 to 20, from 15 to 25, from 20 to 30, from 25 to 35, from 30 to 40, from 35 to 45, from 40 to 50, from 45 to 55, from 50 to 60, from 55 to 65, from 60 to 70, from 65 to 75, from 70 to 80, from 75 to 85, from 80 to 90, from 85 to 95, from 90 to 100, from 95 to 105, from 100 to 110, from 105 to 115, from 110 to 120, from 115 to 125, from 120 to 130, from 125 to 135, from 130 to 140, from 135 to 145, from 140 to 150, from 145 to 155, from 150 to 160, from 155 to 165, from 160 to 170, from 165 to 175, from 170 to 180, from 175 to 185, from 180 to 190, from 185 to 195, from 190 to 200, from 195 to 205, from 200 to 210, from 205 to 215, from 210 to 220, from 215 to 225, from 220 to 230, from 225 to 235, from 230 to 240, from 235 to 245, from 240 to 250, from 245 to 255, from 250 to 260, from 255 to 265, from 260 to 270, from 265 to 275, from 270 to 280, from 275 to 285, from 280 to 290, from 285 to 295, from 290 to 300, from 295 to 305, from 300 to 310, from 305 to 315, from 310 to 320, from 315 to 325, from 320 to 330, from 325 to 335, from 330 to 340, from 335 to 345, from 340 to 350, from 345 to 355, from 350 to 360, from 355 to 365, from 360 to 370, from 365 to 375, from 370 to 380, from 375 to 385, from 380 to 390, from 385 to 395, from 390 to 400, from 395 to 405, from 400 to 410, from 405 to 415, from 410 to 420, from 415 to 425, from 420 to 430, from 425 to 435, from 430 to 440, from 435 to 445, from 440 to 450, from 445 to 455, from 450 to 460, from 455 to 465, from 460 to 470, from 465 to 475, from 470 to 480, from 475 to 485, from 480 to 490, from 485 to 495, or from 490 to 500 unique or distinct first sample identification sequences. In some cases, a plurality of second primers can comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, or more individually unique or distinct second sample identification sequences. In some instances, a plurality of second primers can also comprise from 1 to 10, from 5 to 15, from 10 to 20, from 15 to 25, from 20 to 30, from 25 to 35, from 30 to 40, from 35 to 45, from 40 to 50, from 45 to 55, from 50 to 60, from 55 to 65, from 60 to 70, from 65 to 75, from 70 to 80, from 75 to 85, from 80 to 90, from 85 to 95, from 90 to 100, from 95 to 105, from 100 to 110, from 105 to 115, from 110 to 120, from 115 to 125, from 120 to 130, from 125 to 135, from 130 to 140, from 135 to 145, from 140 to 150, from 145 to 155, from 150 to 160, from 155 to 165, from 160 to 170, from 165 to 175, from 170 to 180, from 175 to 185, from 180 to 190, from 185 to 195, from 190 to 200, from 195 to 205, from 200 to 210, from 205 to 215, from 210 to 220, from 215 to 225, from 220 to 230, from 225 to 235, from 230 to 240, from 235 to 245, from 240 to 250, from 245 to 255, from 250 to 260, from 255 to 265, from 260 to 270, from 265 to 275, from 270 to 280, from 275 to 285, from 280 to 290, from 285 to 295, from 290 to 300, from 295 to 305, from 300 to 310, from 305 to 315, from 310 to 320, from 315 to 325, from 320 to 330, from 325 to 335, from 330 to 340, from 335 to 345, from 340 to 350, from 345 to 355, from 350 to 360, from 355 to 365, from 360 to 370, from 365 to 375, from 370 to 380, from 375 to 385, from 380 to 390, from 385 to 395, from 390 to 400, from 395 to 405, from 400 to 410, from 405 to 415, from 410 to 420, from 415 to 425, from 420 to 430, from 425 to 435, from 430 to 440, from 435 to 445, from 440 to 450, from 445 to 455, from 450 to 460, from 455 to 465, from 460 to 470, from 465 to 475, from 470 to 480, from 475 to 485, from 480 to 490, from 485 to 495, or from 490 to 500 unique or distinct second sample identification sequences. A plurality of first primers can comprise 96 first sample identification sequences. A plurality of second primers can comprise 96 second sample identification sequences. In some cases, a plurality of first primers can comprise 96 first sample identification sequences, and a plurality of second primers can comprise 96 second sample identification sequences.

FIG. 13 shows another example schematic corresponding to the preceding example. Panel 1300 shows a workflow corresponding to processing of chromatin from a cell, cell bead, or cell nucleus.

As shown in panel 1300, in bulk solution, chromatin included within a cell, cell bead, or cell nucleus is processed (e.g., as described herein) to provide a template nucleic acid fragment (e.g., tagmented fragment or tagged DNA fragment) 1304 comprising insert sequence 1308 (e.g., region of open chromatin) and a complement thereof, transposon end sequences 1306 and complements thereof, first tag sequence (e.g., sequencing primer) or portion thereof 1302 (e.g., an R1 or R2 sequence), second tag sequence (e.g., sequencing primer) or portion thereof 1310 (e.g., an R1 or R2 sequence), and gaps 1307. Template nucleic acid fragment 1304 may then be partitioned within a partition (e.g., a droplet or well, as described herein). Within the partition, the cell, cell bead, or cell nucleus comprising template nucleic acid fragment 1304 may be lysed, permeabilized, or otherwise processed to provide access to template nucleic acid fragment 1304 (and one or more RNA molecules) therein. Gaps 1307 may be filled 1312 via a gap filling extension process (e.g., using a DNA polymerase). The partition may include a bead (e.g., gel bead) 1316a coupled to a nucleic acid barcode molecule 1318a. Nucleic acid barcode molecule 1318a may comprise a spacer sequence (e.g., first primer binding sequence) 1320a, a barcode sequence 1322a, and a tag binding sequence (e.g., sequencing primer) or portion thereof or complement thereof 1302′. Sequence 1302′ may hybridize to sequence 1302 of template nucleic acid fragment 1304, or its complement, and undergo primer extension 1314 to yield a single-stranded sequence 1326 comprising sequences 1320a, 1322a, 1302′, 1310, and insert sequence 1308 or a complement thereof. The contents of the partition may then be recovered in bulk solution (e.g., a droplet may be broken) to provide the strand in bulk solution. Sequence 1326 is then dual-indexed with a ligation reaction 1328 using a pair of ligation indexing adaptors 1330a and 1330d. 1330a may comprise a first sample identification sequence 1330b (e.g., an i5 index sequence) and a first flow cell adapter sequence 1330c (e.g., a P5 sequence). 1330d may comprise a second sample identification sequence 1330e (e.g., an i7 index sequence) and a second flow cell adapter sequence 1330e (e.g., a P7 sequence). In some embodiments, the ligation reaction can be performed using a splint molecule comprising a sequence complementary to the spacer sequence (e.g., first primer binding sequence) 1320a or a portion thereof. In some embodiments, the ligation reaction can be performed using a splint molecule comprising a sequence complementary to the second tag sequence (e.g., sequencing primer) 1310 or a portion thereof. In some cases, the splint molecule may also comprise a sequence complementary to a sequence comprised by the indexing adaptors 1330a or 1330d. The ligation product of 1332 may include sequences of the nucleic acid barcode molecule 1318a, the original chromatin molecule, the first and second sample identification sequence 1330b and 1330e, and the first and second flow cell adapter sequence 1330c and 1330f. Sequence 1332 then undergoes another amplification 1334 (e.g., PCR) to generate a double-stranded sequence 1336.

In some instances, a ligation indexing adaptor may comprise, from 5′ to 3′, a flow cell adaptor sequence or any complements thereof (e.g., a P5 or P7 sequence), and a sample identification sequence or any complements thereof (e.g., an i5 or i7 index sequence). In one example, a first ligation indexing adaptor may comprise, from 5′ to 3′, a flow cell adaptor sequence or any complements thereof (e.g., a P5 sequence) and a sample identification sequence or any complements thereof (e.g., an i5 index sequence). In another example, a second ligation indexing adaptor may comprise, from 5′ to 3′, a flow cell adaptor sequence or any complements thereof (e.g., a P7 sequence) and a sample identification sequence or any complements thereof (e.g., an i7 index sequence). These nucleotides may be completely contiguous, e.g., in a single stretch of adjacent nucleotides, or they may be separated into two or more separate subsequences that are separated by 1 or more nucleotides.

In some cases, a plurality of first ligation indexing adaptors can comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, or more individually unique or distinct first sample identification sequences. In some instances, a plurality of first ligation indexing adaptors can also comprise from 1 to 10, from 5 to 15, from 10 to 20, from 15 to 25, from 20 to 30, from 25 to 35, from 30 to 40, from 35 to 45, from 40 to 50, from 45 to 55, from 50 to 60, from 55 to 65, from 60 to 70, from 65 to 75, from 70 to 80, from 75 to 85, from 80 to 90, from 85 to 95, from 90 to 100, from 95 to 105, from 100 to 110, from 105 to 115, from 110 to 120, from 115 to 125, from 120 to 130, from 125 to 135, from 130 to 140, from 135 to 145, from 140 to 150, from 145 to 155, from 150 to 160, from 155 to 165, from 160 to 170, from 165 to 175, from 170 to 180, from 175 to 185, from 180 to 190, from 185 to 195, from 190 to 200, from 195 to 205, from 200 to 210, from 205 to 215, from 210 to 220, from 215 to 225, from 220 to 230, from 225 to 235, from 230 to 240, from 235 to 245, from 240 to 250, from 245 to 255, from 250 to 260, from 255 to 265, from 260 to 270, from 265 to 275, from 270 to 280, from 275 to 285, from 280 to 290, from 285 to 295, from 290 to 300, from 295 to 305, from 300 to 310, from 305 to 315, from 310 to 320, from 315 to 325, from 320 to 330, from 325 to 335, from 330 to 340, from 335 to 345, from 340 to 350, from 345 to 355, from 350 to 360, from 355 to 365, from 360 to 370, from 365 to 375, from 370 to 380, from 375 to 385, from 380 to 390, from 385 to 395, from 390 to 400, from 395 to 405, from 400 to 410, from 405 to 415, from 410 to 420, from 415 to 425, from 420 to 430, from 425 to 435, from 430 to 440, from 435 to 445, from 440 to 450, from 445 to 455, from 450 to 460, from 455 to 465, from 460 to 470, from 465 to 475, from 470 to 480, from 475 to 485, from 480 to 490, from 485 to 495, or from 490 to 500 unique or distinct first sample identification sequences. In some cases, a plurality of second ligation indexing adaptors can comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, or more individually unique or distinct second sample identification sequences. In some instances, a plurality of second ligation indexing adaptors can also comprise from 1 to 10, from 5 to 15, from 10 to 20, from 15 to 25, from 20 to 30, from 25 to 35, from 30 to 40, from 35 to 45, from 40 to 50, from 45 to 55, from 50 to 60, from 55 to 65, from 60 to 70, from 65 to 75, from 70 to 80, from 75 to 85, from 80 to 90, from 85 to 95, from 90 to 100, from 95 to 105, from 100 to 110, from 105 to 115, from 110 to 120, from 115 to 125, from 120 to 130, from 125 to 135, from 130 to 140, from 135 to 145, from 140 to 150, from 145 to 155, from 150 to 160, from 155 to 165, from 160 to 170, from 165 to 175, from 170 to 180, from 175 to 185, from 180 to 190, from 185 to 195, from 190 to 200, from 195 to 205, from 200 to 210, from 205 to 215, from 210 to 220, from 215 to 225, from 220 to 230, from 225 to 235, from 230 to 240, from 235 to 245, from 240 to 250, from 245 to 255, from 250 to 260, from 255 to 265, from 260 to 270, from 265 to 275, from 270 to 280, from 275 to 285, from 280 to 290, from 285 to 295, from 290 to 300, from 295 to 305, from 300 to 310, from 305 to 315, from 310 to 320, from 315 to 325, from 320 to 330, from 325 to 335, from 330 to 340, from 335 to 345, from 340 to 350, from 345 to 355, from 350 to 360, from 355 to 365, from 360 to 370, from 365 to 375, from 370 to 380, from 375 to 385, from 380 to 390, from 385 to 395, from 390 to 400, from 395 to 405, from 400 to 410, from 405 to 415, from 410 to 420, from 415 to 425, from 420 to 430, from 425 to 435, from 430 to 440, from 435 to 445, from 440 to 450, from 445 to 455, from 450 to 460, from 455 to 465, from 460 to 470, from 465 to 475, from 470 to 480, from 475 to 485, from 480 to 490, from 485 to 495, or from 490 to 500 unique or distinct second sample identification sequences. A plurality of first ligation indexing adaptors can comprise 96 first sample identification sequences. A plurality of second ligation indexing adaptors can comprise 96 second sample identification sequences. In some cases, a plurality of first ligation indexing adaptors can comprise 96 first sample identification sequences, and a plurality of second ligation indexing adaptors can comprise 96 second sample identification sequences.

In another example, a cell, cell bead, or cell nucleus comprising chromatin is provided. The chromatin in the cell, cell bead, or cell nucleus may be processed to provide a template nucleic acid fragment derived from the chromatin (e.g., a tagmented fragment, as described herein). The chromatin may be processed in bulk solution. For example, a transposase-nucleic acid complex such as that shown in FIG. 9 may be used to prepare the template nucleic acid fragment. The template nucleic acid fragment may be at least partially double-stranded. The template nucleic acid fragment may comprise a double-stranded region comprising sequences of chromatin of the cell, cell bead, or cell nucleus. A first end of a first strand of the double-stranded region may be linked to a first transposon end sequence (e.g., mosaic end sequence), which first transposon end sequence may be linked to a first tag sequence (e.g., a first sequencing primer) or portion thereof. A first end of the second strand of the double-stranded region, which end is opposite the first end of the first strand, may be linked to a second transposon end sequence (e.g., mosaic end sequence), which second transposon end sequence may be linked to a second tag sequence (e.g., a second sequencing primer) or portion thereof. The second transposon end sequence may be the same as or different from the first transposon end sequence. The first sequencing primer or portion thereof may be the same as or different from the second sequencing primer or portion thereof. In some cases, the first sequencing primer or portion thereof may be an R1 sequence or portion thereof, and the second sequencing primer or portion thereof may be an R2 sequence or portion thereof. The first transposon end sequence may be hybridized to a first complementary sequence (e.g., mosaic end reverse complement sequence), which first complementary sequence may not be linked to a second end of the second strand of the double-stranded region of the first template nucleic acid fragment. Similarly, the second transposon end sequence may be hybridized to a second complementary sequence (e.g., mosaic end reverse complement sequence), which second complementary sequence may not be linked to a second end of the first strand of the double-stranded region of the first template nucleic acid fragment. In other words, the first template nucleic acid fragment may comprise one or more gaps. In some cases, the one or more gaps may be approximately 9 bp in length each. For example, one or more gaps may be at least about 1, 2, 3, 4, 5, 6, 7, 8, 9, 10, or more bp in length. For example, one or more gaps may be at most about 10, 9, 8, 7, 6, 5, 4, 3, 2, or 1 bp in length. The primer molecule may also comprise an additional primer sequence.

The partially double-stranded molecule corresponding to the chromatin of the cell, cell bead, or cell nucleus included within the partition (e.g., droplet or well) of the plurality of partitions may be recovered from the partition. For example, the contents of the plurality of partitions may be pooled to provide these products in a bulk solution.

Outside of the partition, the gaps in the partially double-stranded nucleic acid molecule corresponding to the chromatin may be filled using via a gap filling extension process (e.g., using a DNA polymerase or reverse transcriptase). The gap filling extension process may not include strand displacement. The resultant gap-filled double-stranded nucleic acid molecule may be denatured to provide a single strand, which single strand may be subjected to conditions sufficient to perform one or more nucleic acid amplification reactions (e.g., PCR) to generate amplification products corresponding to the chromatin of the cell, cell bead, or cell nucleus. A nucleic acid amplification process may incorporate one or more additional sequences, such as one or more additional flow cell adapter sequences.

A dual-indexed nucleic acid molecule may comprise, from 5′ to 3′, a first flow cell adaptor sequence or any complements thereof, a first sample identification sequence or any complements thereof, a first primer sequence or complement thereof, a template nucleic acid fragment (e.g., tagmented fragment or tagged DNA fragment), a second primer sequence or complement thereof, a second sample identification sequence or any complements thereof, and a second flow cell adaptor sequence or any complements thereof. In some embodiments, a generated nucleic acid molecule that is barcoded and dual indexed may comprise: a first flow cell adaptor sequence (e.g., a P5 sequence)-a first sample identification sequence (e.g., an i5 index sequence)-a first primer sequence (e.g., a spacer sequence)-a barcode sequence-a first sequencing primer/tag sequence (e.g., R1 or R2 sequence)-a tagmented fragment)-a second sequencing primer/tag sequence (e.g., R1 or R2 sequence)-a second sample identification sequence (e.g., an i7 index sequence)-a second flow cell adaptor sequence, or a complementary sequence of any of the sequences thereof. In some cases, a dual-indexed nucleic acid molecule may comprise, from 5′ to 3′, a first flow cell adaptor sequence or any complements thereof (e.g., a P5 sequence), a first sample identification sequence or any complements thereof (e.g., an i5 sequence), a first primer sequence or complement thereof (e.g., a spacer sequence), a first tag sequence or complement thereof (e.g., an R1 or R2 sequence), a template nucleic acid fragment (e.g., tagmented fragment or tagged DNA fragment), a second tag sequence or complement thereof (e.g., an R1 or R2 sequence), a second sample identification sequence (e.g., an i7 sequence) or any complements thereof, and a second flow cell adaptor sequence (e.g., a P7 sequence) or any complements thereof. A dual-indexed nucleic acid molecule may also comprise, from 5′ to 3′, a second flow cell adaptor sequence or any complements thereof (e.g., a P7 sequence), a second sample identification sequence or any complements thereof (e.g., an i7 sequence), a second tag sequence or complement thereof (e.g., an R1 or R2 sequence), a template nucleic acid fragment (e.g., tagmented fragment), a first tag sequence (e.g., an R1 or R2 sequence), a first primer sequence or complement thereof (e.g., a spacer sequence), a first sample identification sequence (e.g., an i5 sequence) or any complements thereof, and a first flow cell adaptor sequence (e.g., a P5 sequence) or any complements thereof. These nucleotides may be completely contiguous, e.g., in a single stretch of adjacent nucleotides, or they may be separated into two or more separate subsequences that are separated by 1 or more nucleotides.

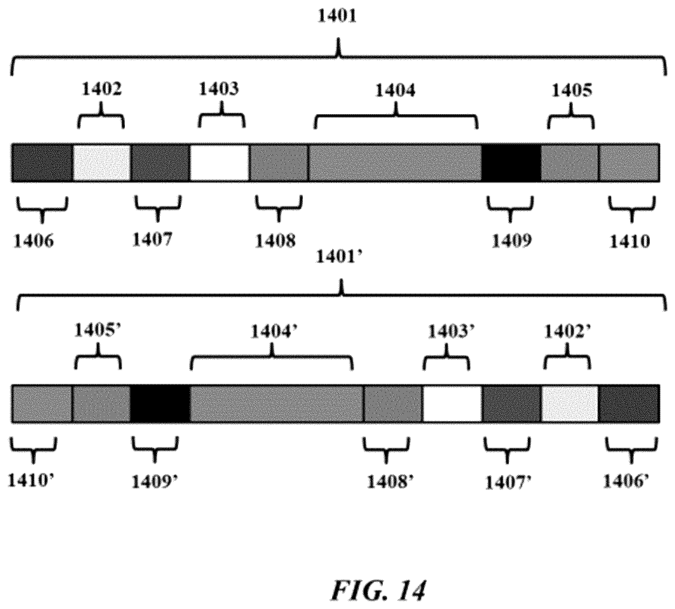

FIG. 14 shows two examples of nucleic acid molecule that is barcoded and comprising a pair of distinct sample identification sequences (e.g., dual indexed). 1401 comprises, from 5′ to 3′, a first flow cell adaptor sequence 1406 (e.g., a P5 sequence), a first sample identification sequence 1402 (e.g., an i5 index sequence), a first primer sequence 1407 (e.g., a spacer sequence), a barcode sequence 1403, a first sequencing primer/tag sequence 1408 (e.g., an R1 or R2 sequence), a template fragment 1404 (e.g., a tagmented fragment), a second sequencing primer/tag sequence 1409 (e.g., an R1 or R2 sequence), a second sample identification sequence 1405 (e.g., an i7 index sequence), and a second flow cell adaptor sequence 1410 (e.g., a P7 sequence). 1401′ comprises, from 5′ to 3′, a second flow cell adaptor sequence complement 1410′ (e.g., a P7 sequence complement), a second sample identification sequence complement 1405′ (e.g., an i7 index sequence complement), a second sequencing primer/tag sequence complement 1409′ (e.g., an R1 or R2 sequence complement), a template fragment complement 1404′ (e.g., a tagmented fragment complement), a first sequencing primer/tag sequence complement 1408′ (e.g., an R1 or R2 sequence complement), a barcode sequence complement 1403′, a first primer sequence complement 1407′ (e.g., a spacer sequence complement), a first sample identification sequence complement 1402′ (e.g., i5 index sequence complement), and a first flow cell adaptor sequence complement 1406′ (e.g., a P5 sequence complement). In some embodiments, the barcoded and dual indexed nucleic acid molecule comprises the tagmented DNA fragment flanked by a pair of sequencing primer/tag sequences that comprises R1 and R1, R1 and R2, or R2 and R2 (or complementary sequences thereof).

In some cases, a barcoded, dual-indexed nucleic acid can comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or more pairs of first sample identification sequence and second sample identification sequence. In some instances, a barcoded, dual-indexed nucleic acid can comprise 10×10, 11×11, 12×12, 13×13, 14×14, 15×15, 16×16, 17×17, 18×18, 19×19, 20×20, 21×21, 22×22, 23×23, 24×24, 25×25, 26×26, 27×27, 28×28, 29×29, 30×30, 31×31, 32×32, 33×33, 34×34, 35×35, 36×36, 37×37, 38×38, 39×39, 40×40, 41×41, 42×42, 43×43, 44×44, 45×45, 46×46, 47×47, 48×48, 49×49, 50×50, 51×51, 52×52, 53×53, 54×54, 55×55, 56×56, 57×57, 58×58, 59×59, 60×60, 61×61, 62×62, 63×63, 64×64, 65×65, 66×66, 67×67, 68×68, 69×69, 70×70, 71×71, 72×72, 73×73, 74×74, 75×75, 76×76, 77×77, 78×78, 79×79, 80×80, 81×81, 82×82, 83×83, 84×84, 85×85, 86×86, 87×87, 88×88, 89×89, 90×90, 91×91, 92×92, 93×93, 94×94, 95×95, 96×96, 97×97, 98×98, 99×99, 100×100, 101×101, 102×102, 103×103, 104×104, 105×105, 106×106, 107×107, 108×108, 109×109, 110×110, 111×111, 112×112, 113×113, 114×114, 115×115, 116×116, 117×117, 118×118, 119×119, 120×120, 121×121, 122×122, 123×123, 124×124, 125×125, 126×126, 127×127, 128×128, 129×129, 130×130, 131×131, 132×132, 133×133, 134×134, 135×135, 136×136, 137×137, 138×138, 139×139, 140×140, 141×141, 142×142, 143×143, 144×144, 145×145, 146×146, 147×147, 148×148, 149×149, 150×150, 151×151, 152×152, 153×153, 154×154, 155×155, 156×156, 157×157, 158×158, 159×159, 160×160, 161×161, 162×162, 163×163, 164×164, 165×165, 166×166, 167×167, 168×168, 169×169, 170×170, 171×171, 172×172, 173×173, 174×174, 175×175, 176×176, 177×177, 178×178, 179×179, 180×180, 181×181, 182×182, 183×183, 184×184, 185×185, 186×186, 187×187, 188×188, 189×189, 190×190, 191×191, 192×192, 193×193, 194×194, 195×195, 196×196, 197×197, 198×198, 199×199, 200×200, 201×201, 202×202, 203×203, 204×204, 205×205, 206×206, 207×207, 208×208, 209×209, 210×210, 211×211, 212×212, 213×213, 214×214, 215×215, 216×216, 217×217, 218×218, 219×219, 220×220, 221×221, 222×222, 223×223, 224×224, 225×225, 226×226, 227×227, 228×228, 229×229, 230×230, 231×231, 232×232, 233×233, 234×234, 235×235, 236×236, 237×237, 238×238, 239×239, 240×240, 241×241, 242×242, 243×243, 244×244, 245×245, 246×246, 247×247, 248×248, 249×249, 250×250, 251×251, 252×252, 253×253, 254×254, 255×255, 256×256, 257×257, 258×258, 259×259, 260×260, 261×261, 262×262, 263×263, 264×264, 265×265, 266×266, 267×267, 268×268, 269×269, 270×270, 271×271, 272×272, 273×273, 274×274, 275×275, 276×276, 277×277, 278×278, 279×279, 280×280, 281×281, 282×282, 283×283, 284×284, 285×285, 286×286, 287×287, 288×288, 289×289, 290×290, 291×291, 292×292, 293×293, 294×294, 295×295, 296×296, 297×297, 298×298, 299×299, 300×300, 301×301, 302×302, 303×303, 304×304, 305×305, 306×306, 307×307, 308×308, 309×309, 310×310, 311×311, 312×312, 313×313, 314×314, 315×315, 316×316, 317×317, 318×318, 319×319, 320×320, 321×321, 322×322, 323×323, 324×324, 325×325, 326×326, 327×327, 328×328, 329×329, 330×330, 331×331, 332×332, 333×333, 334×334, 335×335, 336×336, 337×337, 338×338, 339×339, 340×340, 341×341, 342×342, 343×343, 344×344, 345×345, 346×346, 347×347, 348×348, 349×349, 350×350, 351×351, 352×352, 353×353, 354×354, 355×355, 356×356, 357×357, 358×358, 359×359, 360×360, 361×361, 362×362, 363×363, 364×364, 365×365, 366×366, 367×367, 368×368, 369×369, 370×370, 371×371, 372×372, 373×373, 374×374, 375×375, 376×376, 377×377, 378×378, 379×379, 380×380, 381×381, 382×382, 383×383, 384×384, 385×385, 386×386, 387×387, 388×388, 389×389, 390×390, 391×391, 392×392, 393×393, 394×394, 395×395, 396×396, 397×397, 398×398, 399×399, 400×400, 401×401, 402×402, 403×403, 404×404, 405×405, 406×406, 407×407, 408×408, 409×409, 410×410, 411×411, 412×412, 413×413, 414×414, 415×415, 416×416, 417×417, 418×418, 419×419, 420×420, 421×421, 422×422, 423×423, 424×424, 425×425, 426×426, 427×427, 428×428, 429×429, 430×430, 431×431, 432×432, 433×433, 434×434, 435×435, 436×436, 437×437, 438×438, 439×439, 440×440, 441×441, 442×442, 443×443, 444×444, 445×445, 446×446, 447×447, 448×448, 449×449, 450×450, 451×451, 452×452, 453×453, 454×454, 455×455, 456×456, 457×457, 458×458, 459×459, 460×460, 461×461, 462×462, 463×463, 464×464, 465×465, 466×466, 467×467, 468×468, 469×469, 470×470, 471×471, 472×472, 473×473, 474×474, 475×475, 476×476, 477×477, 478×478, 479×479, 480×480, 481×481, 482×482, 483×483, 484×484, 485×485, 486×486, 487×487, 488×488, 489×489, 490×490, 491×491, 492×492, 493×493, 494×494, 495×495, 496×496, 497×497, 498×498, 499×499, 500×500, or more pairs of first sample identification sequence and second sample identification sequence. A barcoded, dual-indexed nucleic acid can also comprise from 1×10\hat{ }0 to 1×10\hat{ }2, from 1×10\hat{ }2 to 5×10\hat{ }3, from 1×10\hat{ }3 to 5×10\hat{ }4, from 1×10\hat{ }4 to 5×10\hat{ }5, from 1×10\hat{ }5 to 5×10\hat{ }6, from 1×10\hat{ }6 to 5×10\hat{ }7, from 1×10\hat{ }7 to 5×10\hat{ }8, from 1×10\hat{ }8 to 5×10\hat{ }9, from 1×10\hat{ }9 to 5×10\hat{ }10, from 1×10\hat{ }10 to 5×10\hat{ }11, from 1×10\hat{ }11 to 5×10\hat{ }12, from 1×10\hat{ }12 to 5×10\hat{ }13, from 1×10\hat{ }13 to 5×10\hat{ }14, from 1×10\hat{ }14 to 5×10\hat{ }15, from 1×10\hat{ }15 to 5×10\hat{ }16, from 1×10\hat{ }16 to 5×10\hat{ }17, from 1×10\hat{ }17 to 5×10\hat{ }18, from 1×10\hat{ }18 to 5×10\hat{ }19, from 1×10\hat{ }19 to 5×10\hat{ }20, or more pairs of first sample identification sequence and second sample identification sequence. In other cases, a barcoded, dual-indexed nucleic acid can comprise about 1000 pairs of first sample identification sequence and second sample identification sequence. In other cases, a barcoded, dual-indexed nucleic acid can comprise about 5000 pairs of first sample identification sequence and second sample identification sequence. In other cases, a barcoded, dual-indexed nucleic acid can comprise about 9000 pairs of first sample identification sequence and second sample identification sequence. In other cases, a barcoded, dual-indexed nucleic acid can comprise about 9216 pairs of first sample identification sequence and second sample identification sequence.

Barcoded, dual-indexed nucleic acid molecules may be sequenced in bulk. Methods for processing sequencing read data, such as for ATAC-seq, are described in described in U.S. Patent Publication Nos. US20190040464A1, US20190361010A1, and US20190376058A1, each of which is entirely incorporated herein by reference for all purposes.

### Sequencing and Applications

In some instances, dual indexing a nucleic acid molecule may identify the template or library origin of a sequenced nucleic acid molecule. In some cases, a combination of dual index sequences identified in the sequencing read(s) may be associated with and identify the template sequence (e.g., a tagmented DNA fragment) to a sample from a plurality of samples. In some instances, a sequenced nucleic acid molecule may be associated with a pair of distinct sample identification sequences (e.g., dual index). In some cases, a sequencing read of a nucleic acid molecule may comprise a first sample identification sequence (e.g., an i5 index sequence), a first primer sequence (e.g., a spacer sequence), and a barcode sequence. In some cases, a sequencing read of a nucleic acid molecule may comprise a template fragment (e.g., a tagmented fragment) or portion thereof. In some cases, a sequencing read of a nucleic acid molecule may comprise the tagmented fragment or portion thereof sequenced by using a sequencing primer complementary to the sequencing primer/tag sequence (e.g., an R1 or R2 sequence). In some cases, a sequencing read of a nucleic acid molecule may comprise a second sample identification sequence (e.g., an i7 index sequence). In some cases, a sequencing read comprises a complementary sequence or a portion thereof of any of the sequences described herein. In some cases, a sequencing read of a nucleic acid molecule is generated using a sequencing primer that comprises a sequence complementary to a flow cell adaptor sequence. In some cases, a sequencing read of a nucleic acid molecule is generated using a sequencing primer that comprises a sequence complementary to a sequencing primer/tag sequence (e.g., an R1 or R2 sequence) or portion thereof. In some aspects, a sequencing read of a nucleic acid molecule can be generated using a sequencing primer that binds upstream or downstream of the sample identification sequences or tagmented nucleic acid (e.g., DNA) fragment.

In some instances, one or more sequences of the dual-indexed nucleic acid molecule generated may be determined in a at least one sequencing read using at least one sequencing primer. In some cases, multiple sequencing reads can be generated using multiple sequencing primers. In some instances, a plurality of sequencing reads are generated to identify the template or library origin of a sequenced nucleic acid molecule (e.g., tagmented DNA fragment) and identify the associated cell or cell nucleus. In some cases, at least two, at least three, or at least four sequencing reads are generated for each dual-indexed nucleic acid molecule generated. For example, the plurality of sequencing reads may comprise: a first sequencing read comprising said first sample identification sequence and said barcode sequence or complements thereof; a second sequencing read comprising said second sample identification sequence or a complement thereof; a third sequencing read comprising said tagged DNA fragment sequence or complement a thereof, and a fourth sequencing comprising said tagged DNA fragment sequence or complement a thereof. In some instances comprising a plurality of sequencing reads, the reads may be generated in any suitable order. In some aspects, optionally a first sequencing primer can be removed after a first sequencing read is complete, and repeating the operations for a second, third, and fourth, sequencing read with additional primers. In some instances, each sequencing reaction comprises hybridizing a sequencing primer to a single stranded region of the dual-indexed nucleic acid molecule generated or a product thereof (e.g., amplification product) and identifying the sequence of a region of the template strand. In some cases, a second, third, and/or fourth read may be generated from the same or different end or same or different strand of the nucleic acid fragment (e.g, dual-indexed nucleic acid molecule).

In some cases, the sequence of a first and second sample identification sequence may identify a sample from a plurality of samples. In some instances, each sequence of a plurality of first sample identification sequences is unique. In some cases, each sequence of a plurality of second sample identification sequences is unique. In some cases, a unique sequence of a first sample identification sequence may be paired with a unique sequence of a second sample identification sequence to identify a sample from a plurality of samples. Such pairing may be pre-determined. In other cases, pre-determined pairing can mean that the sequences of a first and second sample identification sequence of a plurality of first and second sample identification sequences assigned to a sample from a plurality of samples in an experiment or analysis is known and/or recorded before the experiment or analysis is carried out.

Each sequence in the pair of sample identification sequences may be introduced to the analyte (e.g., nucleic acid molecule, such as a fragment) sequentially in a controlled manner, such that the same analyte receives the same or intended pair of sample identification sequences, as described in the systems and methods presented herein.

In some cases, sets (e.g., pairs) of sample identification sequences may be assigned to specific different samples. In one example, a first set of first and second sample identification sequences are assigned to a first sample and, a second set of first and second sample identification sequences are assigned to a second sample. A first sequencing read comprising the first and second sample identification sequences from the first set can identify the first sequencing read as having originated from the first sample. A second sequencing read comprising the first and second sample identification sequences from the second set can identify the second sequencing read as having originated from the second sample. A third sequencing read comprising the first sample identification sequence from the first set and the second sample identification sequence not from the first set, or the first sample identification sequence from the second set and the second sample identification sequence not from the second set may not identify the third sequencing read as having originated from any known sample. In some cases, if the sequences of a pair of first and second sample identification sequence are different from those of any pre-determined pairs of first and second sample identification sequence, the sample may be identified as having experienced index hopping during the experiment. In some cases, a sample having experienced index hopping may have misassignment of library origin of a sequenced nucleic acid molecule. A sample having experienced index hopping may not be processed for further analysis. A sample having experienced index hopping may be discarded and filtered. In some cases, a sample having experienced index hopping may be flagged as having index hopped if it is undergoing further analysis. In some cases, sequencing reads comprising the same set of sample identification sequences, without an assignment to a specific sample, may be categorized as its own sample. In some cases, mismatched assignment of sample identification sequences can be identified, filtered out, and/or discarded to prevent confounding results or impacting accuracy of the sequencing. In some aspects, a pair of sample identification sequences from the sequencing read(s) can be compared to the sample identification sequences that were assigned to a sample, which was known or recorded. In some particular embodiments, an unexpected pairing or association of sample identification sequences can be identified.

### Dual-Indexing Kits

Provided herein are kits for dual-indexing nucleic acid molecules and performing any of the methods provided herein. In some cases, a kit may comprise a nucleic acid molecule described herein, such as with respect to FIG. 8, 12, 13, 14, 15, 16, or 17. A nucleic acid barcode molecule may comprise, form 5′ to 3′: (i) a first primer binding sequence, (ii) a barcode sequence, and (iii) a first tag binding sequence. In some cases, a first primer of the nucleic acid barcode molecule may comprise the first primer described herein, such as with respect to FIG. 8, 12, 13, 14, 15, 16, or 17.

The barcode sequence may comprise a barcode sequence as described herein, such as with respect to FIG. 8, 12, 13, 14, 15, 16, or 17. The barcode sequence may comprise a unique molecular identification sequence that is different amongst the plurality of nucleic acid barcode molecules, as described herein.

In some cases, the nucleic acid barcode molecule may be attached to a support described herein, such as with respect to FIG. 8, 12, 13, 14, 15, 16, or 17. The support bead may comprise a bead or a gel bead described herein. In some instances, a support may be coupled to a plurality of nucleic acid barcode molecules described herein, such as with respect to FIG. 8, 12, 13, 14, 15, 16, or 17.

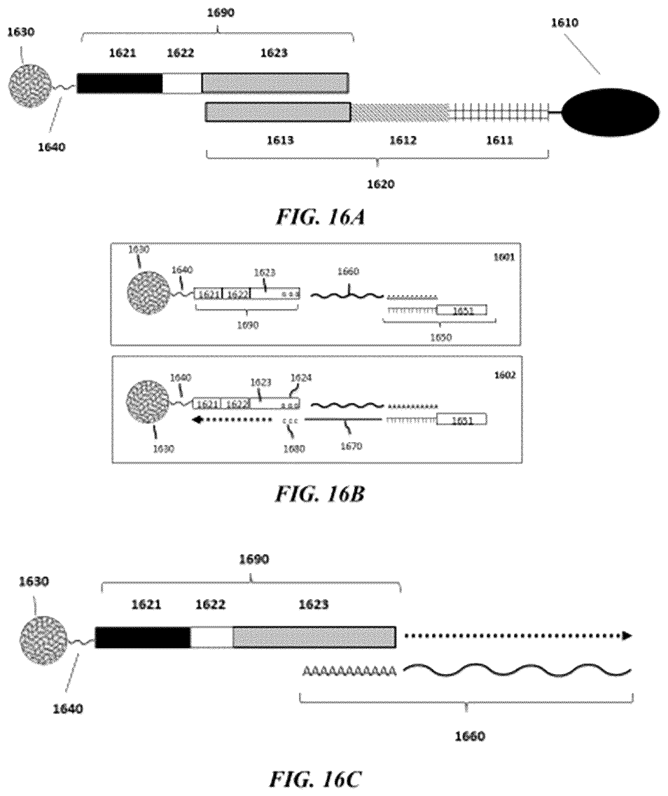

In some cases, a nucleic acid is releasably coupled to the support via a labile moiety described herein, such as with respect to FIG. 16 or 17. The labile moiety may be cleavable via one or more stimuli selected from the group consisting of a thermal, chemical, and photo stimulus, as described herein. In some cases, the labile moiety may comprise a disulfide bond described herein.

In some cases, the nucleic acid barcode molecule may comprise a first tag binding sequence at its 3′ end as described herein, such as with respect to FIG. 8, 12, 13, 14, 15, 16, or 17. The first tag binding sequence may be complementary to a first tag sequence described herein.

In some cases, the kit may further comprise a first additional nucleic acid barcode molecule comprising the first tag sequence or complement thereof and a second additional nucleic acid barcode molecule comprising a second tag sequence different from the first tag sequence, or complement thereof, as described herein, with respected to any one of FIGS. 8-17.

The kit may comprise a first primer molecule as described herein, with respect to FIG. 12 or 13. The first primer may comprise a first adapter sequence described herein. The first adapter sequence may be configured to attach to a flow cell of a sequencer described herein. The kit may also comprise a second primer molecule described herein, with respect to FIG. 12 or 13. The second primer molecule may comprise, from 3′ to 5′: (i) a second tag binding sequence, (ii) the second sample identification sequence, and (iii) a second adapter sequence described herein.

The kit may comprise a plurality of the first primer molecules, each comprising a different first sample identification sequence described herein, such as with respect to FIG. 12 or 13, and the first primer molecule. The plurality of the first primer molecules comprises at least 96 first sample identification sequences.

The kit may comprise a plurality of the second primer molecules, each comprising a different second sample identification sequence described herein, such as with respect to FIG. 12 or 13, and the second primer molecule. The plurality of the second primer molecules comprises at least 96 second sample identification sequences.

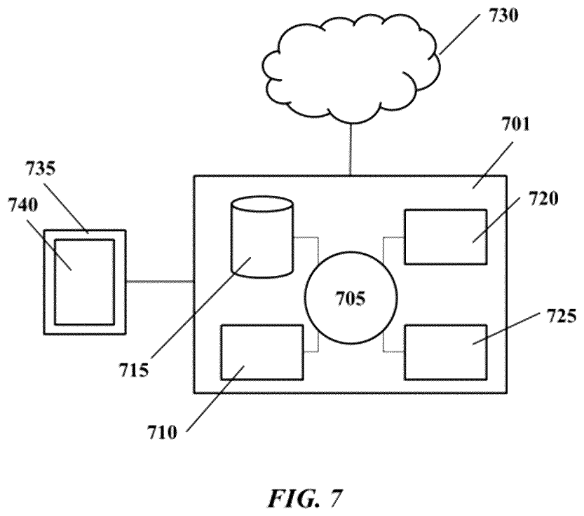

The kit may comprise a record of the plurality of the first sample identification sequences and the second sample identification sequences, as described herein, with respect to FIG. 14. In some instances, the kit may comprise a computer readable medium described herein, with respect to FIG. 7. The computer medium may comprise instructions in memory configured to present to an operator an interface to record a plurality of pairs of a given first primer identification sequence of the plurality of first sample identification sequences and a given second primer identification sequence of the plurality of second sample identification sequences

In some cases, the kit may comprise a transposase described herein, with respect to FIG. 9, 10, or 11. The kit may also comprise an emulsion reagent described herein. One such reagent may comprise a surfactant described herein. In some cases, the kit may also comprise reagents that are configured to permeabilize or fix a cell or cell nucleus, as described herein. In some instances, the kit may also comprise one or more reagents that may include, for example, enzymes (e.g., polymerases, etc.).

### Systems and Methods for Sample Compartmentalization

In an aspect, the systems and methods described herein provide for the compartmentalization, depositing, or partitioning of one or more particles (e.g., biological particles, macromolecular constituents of biological particles, beads, reagents, etc.) into discrete compartments or partitions (referred to interchangeably herein as partitions), where each partition maintains separation of its own contents from the contents of other partitions. The partition can be a droplet in an emulsion or a well. A partition may comprise one or more other partitions.

A partition may include one or more particles. A partition may include one or more types of particles. For example, a partition of the present disclosure may comprise one or more biological particles and/or macromolecular constituents thereof. A partition may comprise one or more beads. A partition may comprise one or more gel beads. A partition may comprise one or more cell beads. A partition may include a single gel bead, a single cell bead, or both a single cell bead and single gel bead. A partition may include one or more reagents. Alternatively, a partition may be unoccupied. For example, a partition may not comprise a bead. A cell bead can be an biological particle and/or one or more of its macromolecular constituents encased inside of a gel or polymer matrix, such as via polymerization of a droplet containing the biological particle and precursors capable of being polymerized or gelled. Unique identifiers, such as barcodes, may be injected into the droplets previous to, subsequent to, or concurrently with droplet generation, such as via a microcapsule (e.g., bead), as described elsewhere herein.

The methods and systems of the present disclosure may comprise methods and systems for generating one or more partitions such as droplets. The droplets may comprise a plurality of droplets in an emulsion. In some examples, the droplets may comprise droplets in a colloid. In some cases, the emulsion may comprise a microemulsion or a nanoemulsion. In some examples, the droplets may be generated with aid of a microfluidic device and/or by subjecting a mixture of immiscible phases to agitation (e.g., in a container). In some cases, a combination of the mentioned methods may be used for droplet and/or emulsion formation.

Droplets can be formed by creating an emulsion by mixing and/or agitating immiscible phases. Mixing or agitation may comprise various agitation techniques, such as vortexing, pipetting, tube flicking, or other agitation techniques. In some cases, mixing or agitation may be performed without using a microfluidic device. In some examples, the droplets may be formed by exposing a mixture to ultrasound or sonication. Systems and methods for droplet and/or emulsion generation by agitation are described in International Application No. PCT/US20/17785, which is entirely incorporated herein by reference for all purposes.

Microfluidic devices or platforms comprising microfluidic channel networks (e.g., on a chip) can be utilized to generate partitions such as droplets and/or emulsions as described herein. Methods and systems for generating partitions such as droplets, methods of encapsulating biological particles, methods of increasing the throughput of droplet generation, and various geometries, architectures, and configurations of microfluidic devices and channels are described in U.S. Patent Publication Nos. 2019/0367997 and 2019/0064173, each of which is entirely incorporated herein by reference for all purposes.

In some examples, individual particles can be partitioned to discrete partitions by introducing a flowing stream of particles in an aqueous fluid into a flowing stream or reservoir of a non-aqueous fluid, such that droplets may be generated at the junction of the two streams/reservoir, such as at the junction of a microfluidic device provided elsewhere herein.

The methods of the present disclosure may comprise generating partitions and/or encapsulating particles, such as analyte carriers or analyte carriers, in some cases, individual analyte carriers such as single cells. In some examples, reagents may be encapsulated and/or partitioned (e.g., co-partitioned with analyte carriers) in the partitions. Various mechanisms may be employed in the partitioning of individual particles. An example may comprise porous membranes through which aqueous mixtures of cells may be extruded into fluids (e.g., non-aqueous fluids).

The partitions can be flowable within fluid streams. The partitions may comprise, for example, micro-vesicles that have an outer barrier surrounding an inner fluid center or core. In some cases, the partitions may comprise a porous matrix that is capable of entraining and/or retaining materials within its matrix. The partitions can be droplets of a first phase within a second phase, wherein the first and second phases are immiscible. For example, the partitions can be droplets of aqueous fluid within a non-aqueous continuous phase (e.g., oil phase). In another example, the partitions can be droplets of a non-aqueous fluid within an aqueous phase. In some examples, the partitions may be provided in a water-in-oil emulsion or oil-in-water emulsion. A variety of different vessels are described in, for example, U.S. Patent Application Publication No. 2014/0155295, which is entirely incorporated herein by reference for all purposes. Emulsion systems for creating stable droplets in non-aqueous or oil continuous phases are described in, for example, U.S. Patent Application Publication No. 2010/0105112, which is entirely incorporated herein by reference for all purposes.

Fluid properties (e.g., fluid flow rates, fluid viscosities, etc.), particle properties (e.g., volume fraction, particle size, particle concentration, etc.), microfluidic architectures (e.g., channel geometry, etc.), and other parameters may be adjusted to control the occupancy of the resulting partitions (e.g., number of biological particles per partition, number of beads per partition, etc.). For example, partition occupancy can be controlled by providing the aqueous stream at a certain concentration and/or flow rate of particles. To generate single biological particle partitions, the relative flow rates of the immiscible fluids can be selected such that, on average, the partitions may contain less than one biological particle per partition in order to ensure that those partitions that are occupied are primarily singly occupied. In some cases, partitions among a plurality of partitions may contain at most one biological particle (e.g., bead, DNA, cell or cellular material). In some instances, the various parameters (e.g., fluid properties, particle properties, microfluidic architectures, etc.) may be selected or adjusted such that a majority of partitions are occupied, for example, allowing for only a small percentage of unoccupied partitions. The flows and channel architectures can be controlled as to ensure a given number of singly occupied partitions, less than a certain level of unoccupied partitions and/or less than a certain level of multiply occupied partitions.

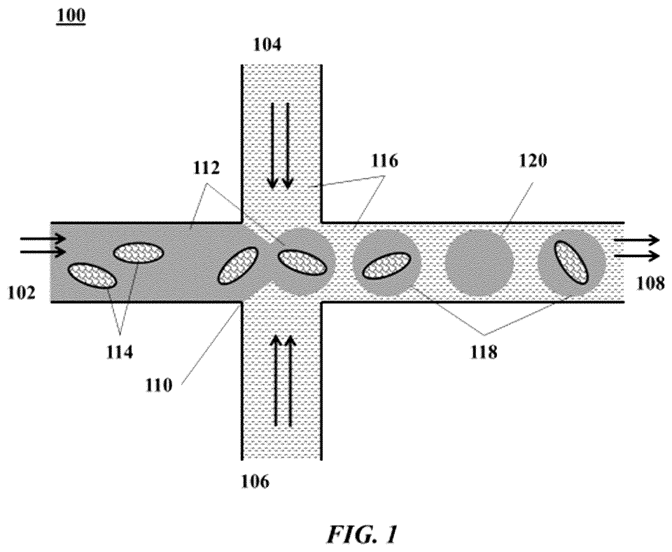

FIG. 1 shows an example of a microfluidic channel structure 100 for partitioning individual biological particles. The channel structure 100 can include channel segments 102, 104, 106 and 108 communicating at a channel junction 110. In operation, a first aqueous fluid 112 that includes suspended biological particles (or cells) 114 may be transported along channel segment 102 into junction 110, while a second fluid 116 that is immiscible with the aqueous fluid 112 is delivered to the junction 110 from each of channel segments 104 and 106 to create discrete droplets 118, 120 of the first aqueous fluid 112 flowing into channel segment 108, and flowing away from junction 110. The channel segment 108 may be fluidically coupled to an outlet reservoir where the discrete droplets can be stored and/or harvested. A discrete droplet generated may include an individual biological particle 114 (such as droplets 118). A discrete droplet generated may include more than one individual biological particle 114 (not shown in FIG. 1). A discrete droplet may contain no biological particle 114 (such as droplet 120). Each discrete partition may maintain separation of its own contents (e.g., individual biological particle 114) from the contents of other partitions.

The second fluid 116 can comprise an oil, such as a fluorinated oil, that includes a fluorosurfactant for stabilizing the resulting droplets, for example, inhibiting subsequent coalescence of the resulting droplets 118, 120. Examples of particularly useful partitioning fluids and fluorosurfactants are described, for example, in U.S. Patent Application Publication No. 2010/0105112, which is entirely incorporated herein by reference for all purposes.

As will be appreciated, the channel segments described herein may be coupled to any of a variety of different fluid sources or receiving components, including reservoirs, tubing, manifolds, or fluidic components of other systems. As will be appreciated, the microfluidic channel structure 100 may have other geometries. For example, a microfluidic channel structure can have more than one channel junction. For example, a microfluidic channel structure can have 2, 3, 4, or 5 channel segments each carrying particles (e.g., biological particles, cell beads, and/or gel beads) that meet at a channel junction. Fluid may be directed to flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

The generated droplets may comprise two subsets of droplets: (1) occupied droplets 118, containing one or more biological particles 114, and (2) unoccupied droplets 120, not containing any biological particles 114. Occupied droplets 118 may comprise singly occupied droplets (having one biological particle) and multiply occupied droplets (having more than one biological particle). As described elsewhere herein, in some cases, the majority of occupied partitions can include no more than one biological particle per occupied partition and some of the generated partitions can be unoccupied (of any biological particle). In some cases, though, some of the occupied partitions may include more than one biological particle. In some cases, the partitioning process may be controlled such that fewer than about 25% of the occupied partitions contain more than one biological particle, and in many cases, fewer than about 20% of the occupied partitions have more than one biological particle, while in some cases, fewer than about 10% or even fewer than about 5% of the occupied partitions include more than one biological particle per partition.

In some cases, the creation of excessive numbers of empty partitions can be minimized, such as to reduce costs and/or increase efficiency. While this minimization may be achieved by providing a sufficient number of biological particles (e.g., biological particles 114) at the partitioning junction 110, such as to ensure that at least one biological particle is encapsulated in a partition, the Poissonian distribution may expectedly increase the number of partitions that include multiple biological particles. As such, where singly occupied partitions are to be obtained, at most about 95%, 90%, 85%, 80%, 75%, 70%, 65%, 60%, 55%, 50%, 45%, 40%, 35%, 30%, 25%, 20%, 15%, 10%, 5% or less of the generated partitions can be unoccupied.

In some cases, the flow of one or more of the biological particles (e.g., in channel segment 102), or other fluids directed into the partitioning junction (e.g., in channel segments 104, 106) can be controlled such that, in many cases, no more than about 50% of the generated partitions, no more than about 25% of the generated partitions, or no more than about 10% of the generated partitions are unoccupied. These flows can be controlled so as to present a non-Poissonian distribution of single-occupied partitions while providing lower levels of unoccupied partitions. The above noted ranges of unoccupied partitions can be achieved while still providing any of the single occupancy rates described above. For example, in many cases, the use of the systems and methods described herein can create resulting partitions that have multiple occupancy rates of less than about 25%, less than about 20%, less than about 15%, less than about 10%, and in many cases, less than about 5%, while having unoccupied partitions of less than about 50%, less than about 40%, less than about 30%, less than about 20%, less than about 10%, less than about 5%, or less.

As will be appreciated, the above-described occupancy rates are also applicable to partitions that include both biological particles and additional reagents, including, but not limited to, microcapsules or beads (e.g., gel beads) carrying barcoded nucleic acid molecules (e.g., oligonucleotides) (described in relation to FIG. 2). The occupied partitions (e.g., at least about 10%, 20%, 30%, 40%, 50%, 60%, 70%, 80%, 90%, 95%, or 99% of the occupied partitions) can include both a microcapsule (e.g., bead) comprising barcoded nucleic acid molecules and a biological particle.

In another aspect, in addition to or as an alternative to droplet based partitioning, biological particles may be encapsulated within a microcapsule that comprises an outer shell, layer or porous matrix in which is entrained one or more individual biological particles or small groups of biological particles. The microcapsule may include other reagents. Encapsulation of biological particles may be performed by a variety of processes. Such processes may combine an aqueous fluid containing the biological particles with a polymeric precursor material that may be capable of being formed into a gel or other solid or semi-solid matrix upon application of a particular stimulus to the polymer precursor. Such stimuli can include, for example, thermal stimuli (e.g., either heating or cooling), photo-stimuli (e.g., through photo-curing), chemical stimuli (e.g., through crosslinking, polymerization initiation of the precursor (e.g., through added initiators)), mechanical stimuli, or a combination thereof.

Preparation of microcapsules comprising biological particles may be performed by a variety of methods. For example, air knife droplet or aerosol generators may be used to dispense droplets of precursor fluids into gelling solutions in order to form microcapsules that include individual biological particles or small groups of biological particles. Likewise, membrane based encapsulation systems may be used to generate microcapsules comprising encapsulated biological particles as described herein. Microfluidic systems of the present disclosure, such as that shown in FIG. 1, may be readily used in encapsulating cells as described herein. In particular, and with reference to FIG. 1, the aqueous fluid 112 comprising (i) the biological particles 114 and (ii) the polymer precursor material (not shown) is flowed into channel junction 110, where it is partitioned into droplets 118, 120 through the flow of non-aqueous fluid 116. In the case of encapsulation methods, non-aqueous fluid 116 may also include an initiator (not shown) to cause polymerization and/or crosslinking of the polymer precursor to form the microcapsule that includes the entrained biological particles. Examples of polymer precursor/initiator pairs include those described in U.S. Patent Application Publication No. 2014/0378345, which is entirely incorporated herein by reference for all purposes.

For example, in the case where the polymer precursor material comprises a linear polymer material, such as a linear polyacrylamide, PEG, or other linear polymeric material, the activation agent may comprise a cross-linking agent, or a chemical that activates a cross-linking agent within the formed droplets. Likewise, for polymer precursors that comprise polymerizable monomers, the activation agent may comprise a polymerization initiator. For example, in certain cases, where the polymer precursor comprises a mixture of acrylamide monomer with a N,N′-bis-(acryloyl)cystamine (BAC) comonomer, an agent such as tetraethylmethylenediamine (TEMED) may be provided within the second fluid streams 116 in channel segments 104 and 106, which can initiate the copolymerization of the acrylamide and BAC into a cross-linked polymer network, or hydrogel.

Upon contact of the second fluid stream 116 with the first fluid stream 112 at junction 110, during formation of droplets, the TEMED may diffuse from the second fluid 116 into the aqueous fluid 112 comprising the linear polyacrylamide, which will activate the crosslinking of the polyacrylamide within the droplets 118, 120, resulting in the formation of gel (e.g., hydrogel) microcapsules, as solid or semi-solid beads or particles entraining the cells 114. Although described in terms of polyacrylamide encapsulation, other ‘activatable’ encapsulation compositions may also be employed in the context of the methods and compositions described herein. For example, formation of alginate droplets followed by exposure to divalent metal ions (e.g., Ca²⁺ ions), can be used as an encapsulation process using the described processes. Likewise, agarose droplets may also be transformed into capsules through temperature based gelling (e.g., upon cooling, etc.).

In some cases, encapsulated biological particles can be selectively releasable from the microcapsule, such as through passage of time or upon application of a particular stimulus, that degrades the microcapsule sufficiently to allow the biological particles (e.g., cell), or its other contents to be released from the microcapsule, such as into a partition (e.g., droplet). For example, in the case of the polyacrylamide polymer described above, degradation of the microcapsule may be accomplished through the introduction of an appropriate reducing agent, such as DTT or the like, to cleave disulfide bonds that cross-link the polymer matrix. See, for example, U.S. Patent Application Publication No. 2014/0378345, which is entirely incorporated herein by reference for all purposes.

The biological particle can be subjected to other conditions sufficient to polymerize or gel the precursors. The conditions sufficient to polymerize or gel the precursors may comprise exposure to heating, cooling, electromagnetic radiation, and/or light. The conditions sufficient to polymerize or gel the precursors may comprise any conditions sufficient to polymerize or gel the precursors. Following polymerization or gelling, a polymer or gel may be formed around the biological particle. The polymer or gel may be diffusively permeable to chemical or biochemical reagents. The polymer or gel may be diffusively impermeable to macromolecular constituents of the biological particle. In this manner, the polymer or gel may act to allow the biological particle to be subjected to chemical or biochemical operations while spatially confining the macromolecular constituents to a region of the droplet defined by the polymer or gel. The polymer or gel may include one or more of disulfide cross-linked polyacrylamide, agarose, alginate, polyvinyl alcohol, polyethylene glycol (PEG)-diacrylate, PEG-acrylate, PEG-thiol, PEG-azide, PEG-alkyne, other acrylates, chitosan, hyaluronic acid, collagen, fibrin, gelatin, or elastin. The polymer or gel may comprise any other polymer or gel.

The polymer or gel may be functionalized to bind to targeted analytes, such as nucleic acids, proteins, carbohydrates, lipids or other analytes. The polymer or gel may be polymerized or gelled via a passive mechanism. The polymer or gel may be stable in alkaline conditions or at elevated temperature. The polymer or gel may have mechanical properties similar to the mechanical properties of the bead. For instance, the polymer or gel may be of a similar size to the bead. The polymer or gel may have a mechanical strength (e.g. tensile strength) similar to that of the bead. The polymer or gel may be of a lower density than an oil. The polymer or gel may be of a density that is roughly similar to that of a buffer. The polymer or gel may have a tunable pore size. The pore size may be chosen to, for instance, retain denatured nucleic acids. The pore size may be chosen to maintain diffusive permeability to exogenous chemicals such as sodium hydroxide (NaOH) and/or endogenous chemicals such as inhibitors. The polymer or gel may be biocompatible. The polymer or gel may maintain or enhance cell viability. The polymer or gel may be biochemically compatible. The polymer or gel may be polymerized and/or depolymerized thermally, chemically, enzymatically, and/or optically.

The polymer may comprise poly(acrylamide-co-acrylic acid) crosslinked with disulfide linkages. The preparation of the polymer may comprise a two-operation reaction. In the first activation operation, poly(acrylamide-co-acrylic acid) may be exposed to an acylating agent to convert carboxylic acids to esters. For instance, the poly(acrylamide-co-acrylic acid) may be exposed to 4-(4,6-dimethoxy-1,3,5-triazin-2-yl)-4-methylmorpholinium chloride (DMTMM). The polyacrylamide-co-acrylic acid may be exposed to other salts of 4-(4,6-dimethoxy-1,3,5-triazin-2-yl)-4-methylmorpholinium. In the second cross-linking operation, the ester formed in the first operation may be exposed to a disulfide crosslinking agent. For instance, the ester may be exposed to cystamine (2,2′-dithiobis(ethylamine)). Following the two operations, the biological particle may be surrounded by polyacrylamide strands linked together by disulfide bridges. In this manner, the biological particle may be encased inside of or comprise a gel or matrix (e.g., polymer matrix) to form a “cell bead.”

A cell bead can contain biological particles (e.g., a cell) or macromolecular constituents (e.g., RNA, DNA, proteins, etc.) of biological particles. A cell bead may include a single cell or multiple cells, or a derivative of the single cell or multiple cells. For example after lysing and washing the cells, inhibitory components from cell lysates can be washed away and the macromolecular constituents can be bound as cell beads. Systems and methods disclosed herein can be applicable to both cell beads (and/or droplets or other partitions) containing biological particles and cell beads (and/or droplets or other partitions) containing macromolecular constituents of biological particles. Cell beads may be or include a cell, cell derivative, cellular material and/or material derived from the cell in, within, or encased in a matrix, such as a polymeric matrix. In some cases, a cell bead may comprise a live cell. In some instances, the live cell may be capable of being cultured when enclosed in a gel or polymer matrix, or of being cultured when comprising a gel or polymer matrix. In some instances, the polymer or gel may be diffusively permeable to certain components and diffusively impermeable to other components (e.g., macromolecular constituents).

Encapsulated biological particles can provide certain potential advantages of being more storable and more portable than droplet-based partitioned biological particles. Furthermore, in some cases, biological particles can be incubated for a select period of time before analysis, such as in order to characterize changes in such biological particles over time, either in the presence or absence of different stimuli (or reagents). In such cases, encapsulation may allow for longer incubation than partitioning in emulsion droplets, although in some cases, droplet partitioned biological particles may also be incubated for different periods of time, e.g., at least 10 seconds, at least 30 seconds, at least 1 minute, at least 5 minutes, at least 10 minutes, at least 30 minutes, at least 1 hour, at least 2 hours, at least 5 hours, or at least 10 hours or more. The encapsulation of biological particles may constitute the partitioning of the biological particles into which other reagents are co-partitioned. Alternatively or in addition, encapsulated biological particles may be readily deposited into other partitions (e.g., droplets) as described above.

### Wells

As described herein, one or more processes may be performed in a partition, which may be a well. The well may be a well of a plurality of wells of a substrate, such as a microwell of a microwell array or plate, or the well may be a microwell or microchamber of a device (e.g., microfluidic device) comprising a substrate. The well may be a well of a well array or plate, or the well may be a well or chamber of a device (e.g., fluidic device). Accordingly, the wells or microwells may assume an “open” configuration, in which the wells or microwells are exposed to the environment (e.g., contain an open surface) and are accessible on one planar face of the substrate, or the wells or microwells may assume a “closed” or “sealed” configuration, in which the microwells are not accessible on a planar face of the substrate. In some instances, the wells or microwells may be configured to toggle between “open” and “closed” configurations. For instance, an “open” microwell or set of microwells may be “closed” or “sealed” using a membrane (e.g., semi-permeable membrane), an oil (e.g., fluorinated oil to cover an aqueous solution), or a lid, as described elsewhere herein.

The well may have a volume of less than 1 milliliter (mL). For instance, the well may be configured to hold a volume of at most 1000 microliters (μL), at most 100 μL, at most 10 μL, at most 1 μL, at most 100 nanoliters (nL), at most 10 nL, at most 1 nL, at most 100 picoliters (pL), at most 10 (pL), or less. The well may be configured to hold a volume of about 1000 μL, about 100 μL, about 10 μL, about 1 μL, about 100 nL, about 10 nL, about 1 nL, about 100 pL, about 10 pL, etc. The well may be configured to hold a volume of at least 10 pL, at least 100 pL, at least 1 nL, at least 10 nL, at least 100 nL, at least 1 μL, at least 10 μL, at least 100 μL, at least 1000 μL, or more. The well may be configured to hold a volume in a range of volumes listed herein, for example, from about 5 nL to about 20 nL, from about 1 nL to about 100 nL, from about 500 pL to about 100 μL, etc. The well may be of a plurality of wells that have varying volumes and may be configured to hold a volume appropriate to accommodate any of the partition volumes described herein.

In some instances, a microwell array or plate comprises a single variety of microwells. In some instances, a microwell array or plate comprises a variety of microwells. For instance, the microwell array or plate may comprise one or more types of microwells within a single microwell array or plate. The types of microwells may have different dimensions (e.g., length, width, diameter, depth, cross-sectional area, etc.), shapes (e.g., circular, triangular, square, rectangular, pentagonal, hexagonal, heptagonal, octagonal, nonagonal, decagonal, etc.), aspect ratios, or other physical characteristics. The microwell array or plate may comprise any number of different types of microwells. For example, the microwell array or plate may comprise 1, 2, 3, 4, 5, 6, 7, 8, 9, 10, 20, 30, 40, 50, 60, 70, 80, 90, 100, 200, 300, 400, 500, 600, 700, 800, 900, 1000 or more different types of microwells. A well may have any dimension (e.g., length, width, diameter, depth, cross-sectional area, volume, etc.), shape (e.g., circular, triangular, square, rectangular, pentagonal, hexagonal, heptagonal, octagonal, nonagonal, decagonal, other polygonal, etc.), aspect ratios, or other physical characteristics described herein with respect to any well.

In certain instances, the microwell array or plate comprises different types of microwells that are located adjacent to one another within the array or plate. For instance, a microwell with one set of dimensions may be located adjacent to and in contact with another microwell with a different set of dimensions. Similarly, microwells of different geometries may be placed adjacent to or in contact with one another. The adjacent microwells may be configured to hold different articles; for example, one microwell may be used to contain a cell, cell bead, or other sample (e.g., cellular components, nucleic acid molecules, etc.) while the adjacent microwell may be used to contain a microcapsule, droplet, bead, or other reagent. In some cases, the adjacent microwells may be configured to merge the contents held within, e.g., upon application of a stimulus, or spontaneously, upon contact of the articles in each microwell.

As is described elsewhere herein, a plurality of partitions may be used in the systems, compositions, and methods described herein. For example, any suitable number of partitions (e.g., wells or droplets) can be generated or otherwise provided. For example, in the case when wells are used, at least about 1,000 wells, at least about 5,000 wells, at least about 10,000 wells, at least about 50,000 wells, at least about 100,000 wells, at least about 500,000 wells, at least about 1,000,000 wells, at least about 5,000,000 wells at least about 10,000,000 wells, at least about 50,000,000 wells, at least about 100,000,000 wells, at least about 500,000,000 wells, at least about 1,000,000,000 wells, or more wells can be generated or otherwise provided. Moreover, the plurality of wells may comprise both unoccupied wells (e.g., empty wells) and occupied wells.

A well may comprise any of the reagents described herein, or combinations thereof. These reagents may include, for example, barcode molecules, enzymes, adapters, and combinations thereof. The reagents may be physically separated from a sample (e.g., a cell, cell bead, or cellular components, e.g., proteins, nucleic acid molecules, etc.) that is placed in the well. This physical separation may be accomplished by containing the reagents within, or coupling to, a microcapsule or bead that is placed within a well. The physical separation may also be accomplished by dispensing the reagents in the well and overlaying the reagents with a layer that is, for example, dissolvable, meltable, or permeable prior to introducing the polynucleotide sample into the well. This layer may be, for example, an oil, wax, membrane (e.g., semi-permeable membrane), or the like. The well may be sealed at any point, for example, after addition of the microcapsule or bead, after addition of the reagents, or after addition of either of these components. The sealing of the well may be useful for a variety of purposes, including preventing escape of beads or loaded reagents from the well, permitting select delivery of certain reagents (e.g., via the use of a semi-permeable membrane), for storage of the well prior to or following further processing, etc.

A well may comprise free reagents and/or reagents encapsulated in, or otherwise coupled to or associated with, microcapsules, beads, or droplets. Any of the reagents described in this disclosure may be encapsulated in, or otherwise coupled to, a microcapsule, droplet, or bead, with any chemicals, particles, and elements suitable for sample processing reactions involving biomolecules, such as, but not limited to, nucleic acid molecules and proteins. For example, a bead or droplet used in a sample preparation reaction for DNA sequencing may comprise one or more of the following reagents: enzymes, restriction enzymes (e.g., multiple cutters), ligase, polymerase, fluorophores, oligonucleotide barcodes, adapters, buffers, nucleotides (e.g., dNTPs, ddNTPs) and the like.

Additional examples of reagents include, but are not limited to: buffers, acidic solution, basic solution, temperature-sensitive enzymes, pH-sensitive enzymes, light-sensitive enzymes, metals, metal ions, magnesium chloride, sodium chloride, manganese, aqueous buffer, mild buffer, ionic buffer, inhibitor, enzyme, protein, polynucleotide, antibodies, saccharides, lipid, oil, salt, ion, detergents, ionic detergents, non-ionic detergents, oligonucleotides, nucleotides, deoxyribonucleotide triphosphates (dNTPs), dideoxyribonucleotide triphosphates (ddNTPs), DNA, RNA, peptide polynucleotides, complementary DNA (cDNA), double stranded DNA (dsDNA), single stranded DNA (ssDNA), plasmid DNA, cosmid DNA, chromosomal DNA, genomic DNA, viral DNA, bacterial DNA, mtDNA (mitochondrial DNA), mRNA, rRNA, tRNA, nRNA, siRNA, snRNA, snoRNA, scaRNA, microRNA, dsRNA, ribozyme, riboswitch and viral RNA, polymerase, ligase, restriction enzymes, proteases, nucleases, protease inhibitors, nuclease inhibitors, chelating agents, reducing agents, oxidizing agents, fluorophores, probes, chromophores, dyes, organics, emulsifiers, surfactants, stabilizers, polymers, water, small molecules, pharmaceuticals, radioactive molecules, preservatives, antibiotics, aptamers, and pharmaceutical drug compounds. As described herein, one or more reagents in the well may be used to perform one or more reactions, including but not limited to: cell lysis, cell fixation, permeabilization, nucleic acid reactions, e.g., nucleic acid extension reactions, amplification, reverse transcription, transposase reactions (e.g., tagmentation), etc.

The wells may be provided as a part of a kit. For example, a kit may comprise instructions for use, a microwell array or device, and reagents (e.g., beads). The kit may comprise any useful reagents for performing the processes described herein, e.g., nucleic acid reactions, barcoding of nucleic acid molecules, sample processing (e.g., for cell lysis, fixation, and/or permeabilization).

In some cases, a well comprises a microcapsule, bead, or droplet that comprises a set of reagents that has a similar attribute (e.g., a set of enzymes, a set of minerals, a set of oligonucleotides, a mixture of different barcode molecules, a mixture of identical barcode molecules). In other cases, a microcapsule, bead, or droplet comprises a heterogeneous mixture of reagents. In some cases, the heterogeneous mixture of reagents can comprise all components necessary to perform a reaction. In some cases, such mixture can comprise all components necessary to perform a reaction, except for 1, 2, 3, 4, 5, or more components necessary to perform a reaction. In some cases, such additional components are contained within, or otherwise coupled to, a different microcapsule, droplet, or bead, or within a solution within a partition (e.g., microwell) of the system.

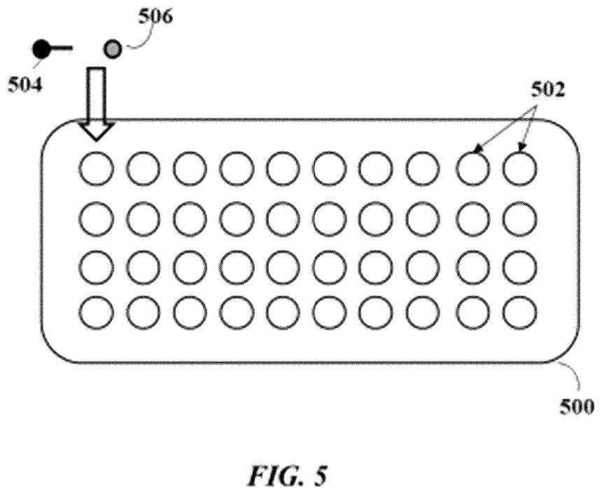

FIG. 5 schematically illustrates an example of a microwell array. The array can be contained within a substrate 500. The substrate 500 comprises a plurality of wells 502. The wells 502 may be of any size or shape, and the spacing between the wells, the number of wells per substrate, as well as the density of the wells on the substrate 500 can be modified, depending on the particular application. In one such example application, a sample molecule 506, which may comprise a cell or cellular components (e.g., nucleic acid molecules) is co-partitioned with a bead 504, which may comprise a nucleic acid barcode molecule coupled thereto. The wells 502 may be loaded using gravity or other loading technique (e.g., centrifugation, liquid handler, acoustic loading, optoelectronic, etc.). In some instances, at least one of the wells 502 contains a single sample molecule 506 (e.g., cell) and a single bead 504.

Reagents may be loaded into a well either sequentially or concurrently. In some cases, reagents are introduced to the device either before or after a particular operation. In some cases, reagents (which may be provided, in certain instances, in microcapsules, droplets, or beads) are introduced sequentially such that different reactions or operations occur at different stages. The reagents (or microcapsules, droplets, or beads) may also be loaded at operations interspersed with a reaction or other operation. For example, microcapsules (or droplets or beads) comprising reagents for fragmenting polynucleotides (e.g., restriction enzymes) and/or other enzymes (e.g., transposases, ligases, polymerases, etc.) may be loaded into the well or plurality of wells, followed by loading of microcapsules, droplets, or beads comprising reagents for attaching nucleic acid barcode molecules to a sample nucleic acid molecule. Reagents may be provided concurrently or sequentially with a sample, e.g., a cell or cellular components (e.g., organelles, proteins, nucleic acid molecules, carbohydrates, lipids, etc.). Accordingly, use of wells may be useful in performing a plurality of operations or reactions.

As described elsewhere herein, the nucleic acid barcode molecules and other reagents may be contained within a microcapsule, bead, or droplet. These microcapsules, beads, or droplets may be loaded into a partition (e.g., a microwell) before, after, or concurrently with the loading of a cell, such that each cell is contacted with a different microcapsule, bead, or droplet. This technique may be used to attach a unique nucleic acid barcode molecule to nucleic acid molecules obtained from each cell. Alternatively or in addition to, the sample nucleic acid molecules may be attached to a support. For instance, the partition (e.g., microwell) may comprise a bead which has coupled thereto a plurality of nucleic acid barcode molecules. The sample nucleic acid molecules, or derivatives thereof, may couple or attach to the nucleic acid barcode molecules on the support. The resulting barcoded nucleic acid molecules may then be removed from the partition, and in some instances, pooled and sequenced. In such cases, the nucleic acid barcode sequences may be used to trace the origin of the sample nucleic acid molecule. For example, polynucleotides with identical barcodes may be determined to originate from the same cell or partition, while polynucleotides with different barcodes may be determined to originate from different cells or partitions.

The samples or reagents may be loaded in the wells or microwells using a variety of approaches. The samples (e.g., a cell, cell bead, or cellular component) or reagents (as described herein) may be loaded into the well or microwell using an external force, e.g., gravitational force, electrical force, magnetic force, or using mechanisms to drive the sample or reagents into the well, e.g., via pressure-driven flow, centrifugation, optoelectronics, acoustic loading, electrokinetic pumping, vacuum, capillary flow, etc. In certain cases, a fluid handling system may be used to load the samples or reagents into the well. The loading of the samples or reagents may follow a Poissonian distribution or a non-Poissonian distribution, e.g., super Poisson or sub-Poisson. The geometry, spacing between wells, density, and size of the microwells may be modified to accommodate a useful sample or reagent distribution; for instance, the size and spacing of the microwells may be adjusted such that the sample or reagents may be distributed in a super-Poissonian fashion.

In one particular non-limiting example, the microwell array or plate comprises pairs of microwells, in which each pair of microwells is configured to hold a droplet (e.g., comprising a single cell) and a single bead (such as those described herein, which may, in some instances, also be encapsulated in a droplet). The droplet and the bead (or droplet containing the bead) may be loaded simultaneously or sequentially, and the droplet and the bead may be merged, e.g., upon contact of the droplet and the bead, or upon application of a stimulus (e.g., external force, agitation, heat, light, magnetic or electric force, etc.). In some cases, the loading of the droplet and the bead is super-Poissonian. In other examples of pairs of microwells, the wells are configured to hold two droplets comprising different reagents and/or samples, which are merged upon contact or upon application of a stimulus. In such instances, the droplet of one microwell of the pair can comprise reagents that may react with an agent in the droplet of the other microwell of the pair. For instance, one droplet can comprise reagents that are configured to release the nucleic acid barcode molecules of a bead contained in another droplet, located in the adjacent microwell. Upon merging of the droplets, the nucleic acid barcode molecules may be released from the bead into the partition (e.g., the microwell or microwell pair that are in contact), and further processing may be performed (e.g., barcoding, nucleic acid reactions, etc.). In cases where intact or live cells are loaded in the microwells, one of the droplets may comprise lysis reagents for lysing the cell upon droplet merging.

A droplet or microcapsule may be partitioned into a well. The droplets may be selected or subjected to pre-processing prior to loading into a well. For instance, the droplets may comprise cells, and only certain droplets, such as those containing a single cell (or at least one cell), may be selected for use in loading of the wells. Such a pre-selection process may be useful in efficient loading of single cells, such as to obtain a non-Poissonian distribution, or to pre-filter cells for a selected characteristic prior to further partitioning in the wells. Additionally, the technique may be useful in obtaining or preventing cell doublet or multiplet formation prior to or during loading of the microwell.

In some instances, the wells can comprise nucleic acid barcode molecules attached thereto. The nucleic acid barcode molecules may be attached to a surface of the well (e.g., a wall of the well). The nucleic acid barcode molecule (e.g., a partition barcode sequence) of one well may differ from the nucleic acid barcode molecule of another well, which can permit identification of the contents contained with a single partition or well. In some cases, the nucleic acid barcode molecule can comprise a spatial barcode sequence that can identify a spatial coordinate of a well, such as within the well array or well plate. In some cases, the nucleic acid barcode molecule can comprise a unique molecular identifier for individual molecule identification. In some instances, the nucleic acid barcode molecules may be configured to attach to or capture a nucleic acid molecule within a sample or cell distributed in the well. For example, the nucleic acid barcode molecules may comprise a capture sequence that may be used to capture or hybridize to a nucleic acid molecule (e.g., RNA, DNA) within the sample. In some instances, the nucleic acid barcode molecules may be releasable from the microwell. For instance, the nucleic acid barcode molecules may comprise a chemical cross-linker which may be cleaved upon application of a stimulus (e.g., photo-, magnetic, chemical, biological, stimulus). The released nucleic acid barcode molecules, which may be hybridized or configured to hybridize to a sample nucleic acid molecule, may be collected and pooled for further processing, which can include nucleic acid processing (e.g., amplification, extension, reverse transcription, etc.) and/or characterization (e.g., sequencing). In such cases, the unique partition barcode sequences may be used to identify the cell or partition from which a nucleic acid molecule originated.

Characterization of samples within a well may be performed. Such characterization can include, in non-limiting examples, imaging of the sample (e.g., cell, cell bead, or cellular components) or derivatives thereof. Characterization techniques such as microscopy or imaging may be useful in measuring sample profiles in fixed spatial locations. For instance, when cells are partitioned, optionally with beads, imaging of each microwell and the contents contained therein may provide useful information on cell doublet formation (e.g., frequency, spatial locations, etc.), cell-bead pair efficiency, cell viability, cell size, cell morphology, expression level of a biomarker (e.g., a surface marker, a fluorescently labeled molecule therein, etc.), cell or bead loading rate, number of cell-bead pairs, etc. In some instances, imaging may be used to characterize live cells in the wells, including, but not limited to: dynamic live-cell tracking, cell-cell interactions (when two or more cells are co-partitioned), cell proliferation, etc. Alternatively or in addition to, imaging may be used to characterize a quantity of amplification products in the well.

In operation, a well may be loaded with a sample and reagents, simultaneously or sequentially. When cells or cell beads are loaded, the well may be subjected to washing, e.g., to remove excess cells from the well, microwell array, or plate. Similarly, washing may be performed to remove excess beads or other reagents from the well, microwell array, or plate. In the instances where live cells are used, the cells may be lysed in the individual partitions to release the intracellular components or cellular analytes. Alternatively, the cells may be fixed or permeabilized in the individual partitions. The intracellular components or cellular analytes may couple to a support, e.g., on a surface of the microwell, on a solid support (e.g., bead), or they may be collected for further downstream processing. For instance, after cell lysis, the intracellular components or cellular analytes may be transferred to individual droplets or other partitions for barcoding. Alternatively, or in addition to, the intracellular components or cellular analytes (e.g., nucleic acid molecules) may couple to a bead comprising a nucleic acid barcode molecule; subsequently, the bead may be collected and further processed, e.g., subjected to nucleic acid reaction such as reverse transcription, amplification, or extension, and the nucleic acid molecules thereon may be further characterized, e.g., via sequencing. Alternatively, or in addition to, the intracellular components or cellular analytes may be barcoded in the well (e.g., using a bead comprising nucleic acid barcode molecules that are releasable or on a surface of the microwell comprising nucleic acid barcode molecules). The barcoded nucleic acid molecules or analytes may be further processed in the well, or the barcoded nucleic acid molecules or analytes may be collected from the individual partitions and subjected to further processing outside the partition. Further processing can include nucleic acid processing (e.g., performing an amplification, extension) or characterization (e.g., fluorescence monitoring of amplified molecules, sequencing). At any convenient or useful stage, the well (or microwell array or plate) may be sealed (e.g., using an oil, membrane, wax, etc.), which enables storage of the assay or selective introduction of additional reagents.

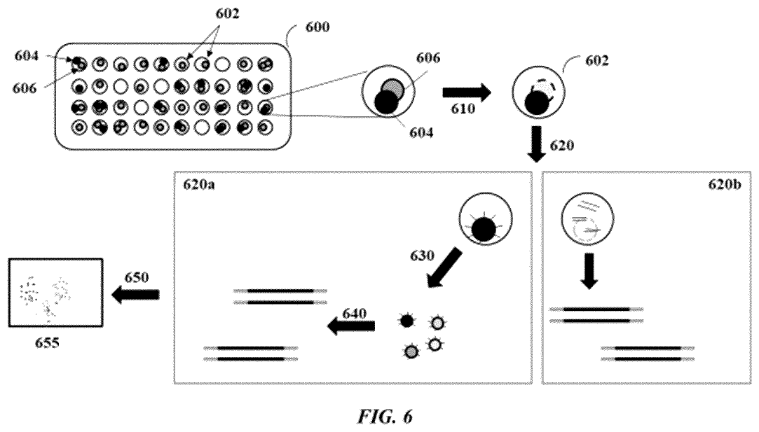

FIG. 6 schematically shows an example workflow for processing nucleic acid molecules within a sample. A substrate 600 comprising a plurality of microwells 602 may be provided. A sample 606 which may comprise a cell, cell bead, cellular components or analytes (e.g., proteins and/or nucleic acid molecules) can be co-partitioned, in a plurality of microwells 602, with a plurality of beads 604 comprising nucleic acid barcode molecules. During process 610, the sample 606 may be processed within the partition. For instance, in the case of live cells, the cell may be subjected to conditions sufficient to lyse the cells and release the analytes contained therein. In process 620, the bead 604 may be further processed. By way of example, processes 620a and 620b schematically illustrate different workflows, depending on the properties of the bead 604.

In 620a, the bead comprises nucleic acid barcode molecules that are attached thereto, and sample nucleic acid molecules (e.g., RNA, DNA) may attach, e.g., via hybridization of ligation, to the nucleic acid barcode molecules. Such attachment may occur on the bead. In process 630, the beads 604 from multiple wells 602 may be collected and pooled. Further processing may be performed in process 640. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 650, further characterization, such as sequencing may be performed to generate sequencing reads. The sequencing reads may yield information on individual cells or populations of cells, which may be represented visually or graphically, e.g., in a plot 655.

In 620b, the bead comprises nucleic acid barcode molecules that are releasably attached thereto, as described below. The bead may degrade or otherwise release the nucleic acid barcode molecules into the well 602; the nucleic acid barcode molecules may then be used to barcode nucleic acid molecules within the well 602. Further processing may be performed either inside the partition or outside the partition. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 650, further characterization, such as sequencing may be performed to generate sequencing reads. The sequencing reads may yield information on individual cells or populations of cells, which may be represented visually or graphically, e.g., in a plot 655.

### Beads

Nucleic acid barcode molecules may be delivered to a partition (e.g., a droplet or well) via a solid support or carrier (e.g., a bead). In some cases, nucleic acid barcode molecules are initially associated with the solid support and then released from the solid support upon application of a stimulus, which allows the nucleic acid barcode molecules to dissociate or to be released from the solid support. In specific examples, nucleic acid barcode molecules are initially associated with the solid support (e.g., bead) and then released from the solid support upon application of a biological stimulus, a chemical stimulus, a thermal stimulus, an electrical stimulus, a magnetic stimulus, and/or a photo stimulus.

A nucleic acid barcode molecule may contain a barcode sequence and a functional sequence, such as a nucleic acid primer sequence or a template switch oligonucleotide (TSO) sequence.

The solid support may be a bead. A solid support, e.g., a bead, may be porous, non-porous, hollow (e.g., a microcapsule), solid, semi-solid, and/or a combination thereof. Beads may be solid, semi-solid, semi-fluidic, fluidic, and/or a combination thereof. In some instances, a solid support, e.g., a bead, may be at least partially dissolvable, disruptable, and/or degradable. In some cases, a solid support, e.g., a bead, may not be degradable. In some cases, the solid support, e.g., a bead, may be a gel bead. A gel bead may be a hydrogel bead. A gel bead may be formed from molecular precursors, such as a polymeric or monomeric species. A semi-solid support, e.g., a bead, may be a liposomal bead. Solid supports, e.g., beads, may comprise metals including iron oxide, gold, and silver. In some cases, the solid support, e.g., the bead, may be a silica bead. In some cases, the solid support, e.g., a bead, can be rigid. In other cases, the solid support, e.g., a bead, may be flexible and/or compressible.

A partition may comprise one or more unique identifiers, such as barcodes. Barcodes may be previously, subsequently or concurrently delivered to the partitions that hold the compartmentalized or partitioned biological particle. For example, barcodes may be injected into droplets or deposited in microwells previous to, subsequent to, or concurrently with droplet generation or providing of reagents in the microwells, respectively. The delivery of the barcodes to a particular partition allows for the later attribution of the characteristics of the individual biological particle to the particular partition. Barcodes may be delivered, for example on a nucleic acid molecule (e.g., an oligonucleotide), to a partition via any suitable mechanism. Barcoded nucleic acid molecules can be delivered to a partition via a microcapsule. A microcapsule, in some instances, can comprise a bead. Beads are described in further detail below.

In some cases, barcoded nucleic acid molecules can be initially associated with the microcapsule and then released from the microcapsule. Release of the barcoded nucleic acid molecules can be passive (e.g., by diffusion out of the microcapsule). In addition or alternatively, release from the microcapsule can be upon application of a stimulus which allows the barcoded nucleic acid molecules to dissociate or to be released from the microcapsule. Such stimulus may disrupt the microcapsule, an interaction that couples the barcoded nucleic acid molecules to or within the microcapsule, or both. Such stimulus can include, for example, a thermal stimulus, photo-stimulus, chemical stimulus (e.g., change in pH or use of a reducing agent(s)), a mechanical stimulus, a radiation stimulus; a biological stimulus (e.g., enzyme), or any combination thereof. Methods and systems for partitioning barcode carrying beads into droplets are provided in US. Patent Publication Nos. 2019/0367997 and 2019/0064173, and International Application No. PCT/US20/17785, each of which is herein entirely incorporated by reference for all purposes.

In some examples, beads, analyte carriers, and droplets may flow along channels (e.g., the channels of a microfluidic device), in some cases at substantially regular flow profiles (e.g., at regular flow rates). Such regular flow profiles may permit a droplet to include a single bead and a single biological particle. Such regular flow profiles may permit the droplets to have an occupancy (e.g., droplets having beads and biological particles) greater than 5%, 10%, 20%, 30%, 40%, 50%, 60%, 70%, 80%, 90%, or 95%. Such regular flow profiles and devices that may be used to provide such regular flow profiles are provided in, for example, U.S. Patent Publication No. 2015/0292988, which is entirely incorporated herein by reference.

A bead may be porous, non-porous, solid, semi-solid, semi-fluidic, fluidic, and/or a combination thereof. In some instances, a bead may be dissolvable, disruptable, and/or degradable. In some cases, a bead may not be degradable. In some cases, the bead may be a gel bead. A gel bead may be a hydrogel bead. A gel bead may be formed from molecular precursors, such as a polymeric or monomeric species. A semi-solid bead may be a liposomal bead. Solid beads may comprise metals including iron oxide, gold, and silver. In some cases, the bead may be a silica bead. In some cases, the bead can be rigid. In other cases, the bead may be flexible and/or compressible.

A bead may be of any suitable shape. Examples of bead shapes include, but are not limited to, spherical, non-spherical, oval, oblong, amorphous, circular, cylindrical, and variations thereof.

Beads may be of uniform size or heterogeneous size. In some cases, the diameter of a bead may be at least about 10 nanometers (nm), 100 nm, 500 nm, 1 micrometer (μm), 5 μm, 10 μm, 20 μm, 30 μm, 40 μm, 50 μm, 60 μm, 70 μm, 80 μm, 90 μm, 100 μm, 250 μm, 500 μm, 1 mm, or greater. In some cases, a bead may have a diameter of less than about 10 nm, 100 nm, 500 nm, 1 μm, 5 μm, 10 μm, 20 μm, 30 μm, 40 μm, 50 μm, 60 μm, 70 μm, 80 μm, 90 μm, 100 μm, 250 μm, 500 μm, 1 mm, or less. In some cases, a bead may have a diameter in the range of about 40-75 μm, 30-75 μm, 20-75 μm, 40-85 μm, 40-95 μm, 20-100 μm, 10-100 μm, 1-100 μm, 20-250 μm, or 20-500 μm.

In certain aspects, beads can be provided as a population or plurality of beads having a relatively monodisperse size distribution. Maintaining relatively consistent bead characteristics, such as size, can contribute to the overall consistency of reagent amounts in partitions. In particular, the beads described herein may have size distributions that have a coefficient of variation in their cross-sectional dimensions of less than 50%, less than 40%, less than 30%, less than 20%, and in some cases less than 15%, less than 10%, less than 5%, or less.

A bead may comprise natural and/or synthetic materials. For example, a bead can comprise a natural polymer, a synthetic polymer or both natural and synthetic polymers. Examples of natural polymers include proteins and sugars such as deoxyribonucleic acid, rubber, cellulose, starch (e.g., amylose, amylopectin), proteins, enzymes, polysaccharides, silks, polyhydroxyalkanoates, chitosan, dextran, collagen, carrageenan, ispaghula, acacia, agar, gelatin, shellac, sterculia gum, xanthan gum, Corn sugar gum, guar gum, gum karaya, agarose, alginic acid, alginate, or natural polymers thereof. Examples of synthetic polymers include acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and/or combinations (e.g., co-polymers) thereof. Beads may also be formed from materials other than polymers, including lipids, micelles, ceramics, glass-ceramics, material composites, metals, other inorganic materials, and others.

In some instances, the bead may contain molecular precursors (e.g., monomers or polymers), which may form a polymer network via polymerization of the molecular precursors. In some cases, a precursor may be an already polymerized species capable of undergoing further polymerization via, for example, a chemical cross-linkage. In some cases, a precursor can comprise one or more of an acrylamide or a methacrylamide monomer, oligomer, or polymer. In some cases, the bead may comprise prepolymers, which are oligomers capable of further polymerization. For example, polyurethane beads may be prepared using prepolymers. In some cases, the bead may contain individual polymers that may be further polymerized together. In some cases, beads may be generated via polymerization of different precursors, such that they comprise mixed polymers, co-polymers, and/or block co-polymers. In some cases, the bead may comprise covalent or ionic bonds between polymeric precursors (e.g., monomers, oligomers, linear polymers), nucleic acid molecules (e.g., oligonucleotides), primers, and other entities. In some cases, the covalent bonds can be carbon-carbon bonds, thioether bonds, or carbon-heteroatom bonds.

Cross-linking may be permanent or reversible, depending upon the particular cross-linker used. Reversible cross-linking may allow for the polymer to linearize or dissociate under appropriate conditions. In some cases, reversible cross-linking may also allow for reversible attachment of a material bound to the surface of a bead. In some cases, a cross-linker may form disulfide linkages. In some cases, the chemical cross-linker forming disulfide linkages may be cystamine or a modified cystamine.

In some cases, disulfide linkages can be formed between molecular precursor units (e.g., monomers, oligomers, or linear polymers) or precursors incorporated into a bead and nucleic acid molecules (e.g., oligonucleotides). Cystamine (including modified cystamines), for example, is an organic agent comprising a disulfide bond that may be used as a crosslinker agent between individual monomeric or polymeric precursors of a bead. Polyacrylamide may be polymerized in the presence of cystamine or a species comprising cystamine (e.g., a modified cystamine) to generate polyacrylamide gel beads comprising disulfide linkages (e.g., chemically degradable beads comprising chemically-reducible cross-linkers). The disulfide linkages may permit the bead to be degraded (or dissolved) upon exposure of the bead to a reducing agent.

In some cases, chitosan, a linear polysaccharide polymer, may be crosslinked with glutaraldehyde via hydrophilic chains to form a bead. Crosslinking of chitosan polymers may be achieved by chemical reactions that are initiated by heat, pressure, change in pH, and/or radiation.

In some cases, a bead may comprise an acrydite moiety, which in certain aspects may be used to attach one or more nucleic acid barcode molecules (e.g., barcode sequence, barcoded nucleic acid molecule, barcoded oligonucleotide, primer, or other oligonucleotide) to the bead. In some cases, an acrydite moiety can refer to an acrydite analogue generated from the reaction of acrydite with one or more species, such as, the reaction of acrydite with other monomers and cross-linkers during a polymerization reaction. Acrydite moieties may be modified to form chemical bonds with a species to be attached, such as a nucleic acid barcode molecule (e.g., barcode sequence, barcoded nucleic acid molecule, barcoded oligonucleotide, primer, or other oligonucleotide). Acrydite moieties may be modified with thiol groups capable of forming a disulfide bond or may be modified with groups already comprising a disulfide bond. The thiol or disulfide (via disulfide exchange) may be used as an anchor point for a species to be attached or another part of the acrydite moiety may be used for attachment. In some cases, attachment can be reversible, such that when the disulfide bond is broken (e.g., in the presence of a reducing agent), the attached species is released from the bead. In other cases, an acrydite moiety can comprise a reactive hydroxyl group that may be used for attachment.

Functionalization of beads for attachment of nucleic acid barcode molecules (e.g., oligonucleotides) may be achieved through a wide range of different approaches, including activation of chemical groups within a polymer, incorporation of active or activatable functional groups in the polymer structure, or attachment at the pre-polymer or monomer stage in bead production.

For example, precursors (e.g., monomers, cross-linkers) that are polymerized to form a bead may comprise acrydite moieties, such that when a bead is generated, the bead also comprises acrydite moieties. The acrydite moieties can be attached to a nucleic acid molecule (e.g., oligonucleotide) that comprises one or more functional sequences, such as a TSO sequence or a primer sequence (e.g., a poly T sequence, or a nucleic acid primer sequence complementary to a target nucleic acid sequence and/or for amplifying a target nucleic acid sequence, a random primer, primer sequence for messenger RNA) and/or one or more barcode sequences. The one more barcode sequences may include sequences that are the same for all nucleic acid barcode molecules coupled to a bead and/or sequences that are different across all nucleic acid barcode molecules coupled to the bead. The nucleic acid barcode molecule may be incorporated into the bead.

In some cases, the nucleic acid barcode molecule can comprise a functional sequence, for example, for attachment to a sequencing flow cell, such as, for example, a P5 sequence (or a portion thereof) for Illumina® sequencing. In some cases, the nucleic acid barcode molecule or derivative thereof (e.g., oligonucleotide or polynucleotide generated from the nucleic acid barcode molecule) can comprise another functional sequence, such as, for example, a P7 sequence (or a portion thereof) for attachment to a sequencing flow cell for Illumina sequencing. In some cases, the nucleic acid barcode molecule can comprise a barcode sequence. In some cases, the nucleic acid barcode molecule can further comprise a unique molecular identifier (UMI). In some cases, the nucleic acid barcode molecule can comprise an R1 primer sequence for Illumina sequencing. In some cases, the nucleic acid barcode molecule can comprise an R2 primer sequence for Illumina sequencing. Examples of such nucleic acid barcode molecules (e.g., oligonucleotides, polynucleotides, etc.) and uses thereof, as may be used with compositions, devices, methods and systems of the present disclosure, are provided in U.S. Patent Pub. Nos. 2014/0378345 and 2015/0376609, each of which is entirely incorporated herein by reference.

In some cases, the nucleic acid barcode molecule can comprise one or more functional sequences. For example, a functional sequence can comprise a sequence for attachment to a sequencing flow cell, such as, for example, a P5 sequence for Illumina® sequencing. In some cases, the nucleic acid barcode molecule or derivative thereof (e.g., oligonucleotide or polynucleotide generated from the nucleic acid barcode molecule) can comprise another functional sequence, such as, for example, a P7 sequence for attachment to a sequencing flow cell for Illumina sequencing. In some cases, the functional sequence can comprise a barcode sequence or multiple barcode sequences. In some cases, the functional sequence can comprise a unique molecular identifier (UMI). In some cases, the functional sequence can comprise a sequencing primer sequence (e.g., an R1 primer sequence for Illumina sequencing, an R2 primer sequence for Illumina sequencing, etc.). In some cases, a functional sequence can comprise a partial sequence, such as a partial barcode sequence, partial anchoring sequence, partial sequencing primer sequence (e.g., partial R1 sequence, partial R2 sequence, etc.), a partial sequence configured to attach to the flow cell of a sequencer (e.g., partial P5 sequence, partial P7 sequence, etc.), or a partial sequence of any other type of sequence described elsewhere herein. A partial sequence may contain a contiguous or continuous portion or segment, but not all, of a full sequence, for example. In some cases, a downstream procedure may extend the partial sequence, or derivative thereof, to achieve a full sequence of the partial sequence, or derivative thereof.

Examples of such nucleic acid barcode molecules (e.g., oligonucleotides, polynucleotides, etc.) and uses thereof, as may be used with compositions, devices, methods and systems of the present disclosure, are provided in U.S. Patent Pub. Nos. 2014/0378345 and 2015/0376609, each of which is entirely incorporated herein by reference.

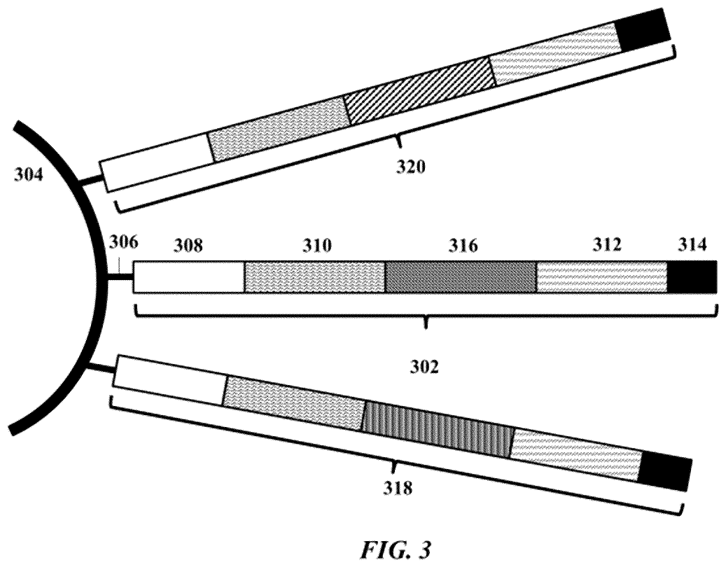

FIG. 3 illustrates an example of a barcode carrying bead. A nucleic acid barcode molecule 302, such as an oligonucleotide, can be coupled to a bead 304 by a releasable linkage 306, such as, for example, a disulfide linker. The same bead 304 may be coupled (e.g., via releasable linkage) to one or more other nucleic acid barcode molecules 318, 320. The nucleic acid barcode molecule 302 may be or comprise a barcode. As noted elsewhere herein, the structure of the barcode may comprise a number of sequence elements. The nucleic acid barcode molecule 302 may comprise a functional sequence 308 that may be used in subsequent processing. For example, the functional sequence 308 may include one or more of a sequencer specific flow cell attachment sequence (e.g., a P5 sequence for Illumina® sequencing systems) and a sequencing primer sequence (e.g., a R1 primer for Illumina® sequencing systems), or partial sequence(s) thereof. The nucleic acid barcode molecule 302 may comprise a barcode sequence 310 for use in barcoding the sample (e.g., DNA, RNA, protein, etc.). In some cases, the barcode sequence 310 can be bead-specific such that the barcode sequence 310 is common to all nucleic acid barcode molecules (e.g., including nucleic acid barcode molecule 302) coupled to the same bead 304. Alternatively or in addition, the barcode sequence 310 can be partition-specific such that the barcode sequence 310 is common to all nucleic acid barcode molecules coupled to one or more beads that are partitioned into the same partition. The nucleic acid barcode molecule 302 may comprise a specific priming sequence 312, such as an mRNA specific priming sequence (e.g., poly-T sequence), a targeted priming sequence, and/or a random priming sequence. The nucleic acid barcode molecule 302 may comprise an anchoring sequence 314 to ensure that the specific priming sequence 312 hybridizes at the sequence end (e.g., of the mRNA). For example, the anchoring sequence 314 can include a random short sequence of nucleotides, such as a 1-mer, 2-mer, 3-mer or longer sequence, which can ensure that a poly-T segment is more likely to hybridize at the sequence end of the poly-A tail of the mRNA.

The nucleic acid barcode molecule 302 may comprise a unique molecular identifying sequence 316 (e.g., unique molecular identifier (UMI)). In some cases, the unique molecular identifying sequence 316 may comprise from about 5 to about 8 nucleotides. Alternatively, the unique molecular identifying sequence 316 may compress less than about 5 or more than about 8 nucleotides. The unique molecular identifying sequence 316 may be a unique sequence that varies across individual nucleic acid barcode molecules (e.g., 302, 318, 320, etc.) coupled to a single bead (e.g., bead 304). In some cases, the unique molecular identifying sequence 316 may be a random sequence (e.g., such as a random N-mer sequence). For example, the UMI may provide a unique identifier of the starting mRNA molecule that was captured, in order to allow quantitation of the number of original expressed RNA. As will be appreciated, although FIG. 3 shows three nucleic acid barcode molecules 302, 318, 320 coupled to the surface of the bead 304, an individual bead may be coupled to any number of individual nucleic acid barcode molecules, for example, from one to tens to hundreds of thousands or even millions of individual nucleic acid barcode molecules. The respective barcodes for the individual nucleic acid barcode molecules can comprise both common sequence segments or relatively common sequence segments (e.g., 308, 310, 312, etc.) and variable or unique sequence segments (e.g., 316) between different individual nucleic acid barcode molecules coupled to the same bead.

In operation, a biological particle (e.g., cell, DNA, RNA, etc.) can be co-partitioned along with a barcode bearing bead 304. The nucleic acid barcode molecules 302, 318, 320 can be released from the bead 304 in the partition. By way of example, in the context of analyzing sample RNA, the poly-T segment (e.g., 312) of one of the released nucleic acid barcode molecules (e.g., 302) can hybridize to the poly-A tail of a mRNA molecule. Reverse transcription may result in a cDNA transcript of the mRNA, but which transcript includes each of the sequence segments 308, 310, 316 of the nucleic acid barcode molecule 302. Because the nucleic acid barcode molecule 302 comprises an anchoring sequence 314, it will more likely hybridize to and prime reverse transcription at the sequence end of the poly-A tail of the mRNA. Within any given partition, all of the cDNA transcripts of the individual mRNA molecules may include a common barcode sequence segment 310. However, the transcripts made from the different mRNA molecules within a given partition may vary at the unique molecular identifying sequence 312 segment (e.g., UMI segment). Beneficially, even following any subsequent amplification of the contents of a given partition, the number of different UMIs can be indicative of the quantity of mRNA originating from a given partition, and thus from the biological particle (e.g., cell). As noted above, the transcripts can be amplified, cleaned up and sequenced to identify the sequence of the cDNA transcript of the mRNA, as well as to sequence the barcode segment and the UMI segment. While a poly-T primer sequence is described, other targeted or random priming sequences may also be used in priming the reverse transcription reaction. Likewise, although described as releasing the barcoded oligonucleotides into the partition, in some cases, the nucleic acid barcode molecules bound to the bead (e.g., gel bead) may be used to hybridize and capture the mRNA on the solid phase of the bead, for example, in order to facilitate the separation of the RNA from other cell contents. In such cases, further processing may be performed, in the partitions or outside the partitions (e.g., in bulk). For instance, the RNA molecules on the beads may be subjected to reverse transcription or other nucleic acid processing, additional adapter sequences may be added to the barcoded nucleic acid molecules, or other nucleic acid reactions (e.g., amplification, nucleic acid extension) may be performed. The beads or products thereof (e.g., barcoded nucleic acid molecules) may be collected from the partitions, and/or pooled together and subsequently subjected to clean up and further characterization (e.g., sequencing).

The operations described herein may be performed at any useful or convenient stage. For instance, the beads comprising nucleic acid barcode molecules may be introduced into a partition (e.g., well or droplet) prior to, during, or following introduction of a sample into the partition. The nucleic acid molecules of a sample may be subjected to barcoding, which may occur on the bead (in cases where the nucleic acid molecules remain coupled to the bead) or following release of the nucleic acid barcode molecules into the partition. In cases where the nucleic acid molecules from the sample remain attached to the bead, the beads from various partitions may be collected, pooled, and subjected to further processing (e.g., reverse transcription, adapter attachment, amplification, clean up, sequencing). In other instances, the processing may occur in the partition. For example, conditions sufficient for barcoding, adapter attachment, reverse transcription, or other nucleic acid processing operations may be provided in the partition and performed prior to clean up and sequencing.

In some instances, a bead may comprise a capture sequence or binding sequence configured to bind to a corresponding capture sequence or binding sequence. In some instances, a bead may comprise a plurality of different capture sequences or binding sequences configured to bind to different respective corresponding capture sequences or binding sequences. For example, a bead may comprise a first subset of one or more capture sequences each configured to bind to a first corresponding capture sequence, a second subset of one or more capture sequences each configured to bind to a second corresponding capture sequence, a third subset of one or more capture sequences each configured to bind to a third corresponding capture sequence, and etc. A bead may comprise any number of different capture sequences. In some instances, a bead may comprise at least 2, 3, 4, 5, 6, 7, 8, 9, 10 or more different capture sequences or binding sequences configured to bind to different respective capture sequences or binding sequences, respectively. Alternatively or in addition, a bead may comprise at most about 10, 9, 8, 7, 6, 5, 4, 3, or 2 different capture sequences or binding sequences configured to bind to different respective capture sequences or binding sequences. In some instances, the different capture sequences or binding sequences may be configured to facilitate analysis of a same type of analyte. In some instances, the different capture sequences or binding sequences may be configured to facilitate analysis of different types of analytes (with the same bead). The capture sequence may be designed to attach to a corresponding capture sequence. Beneficially, such corresponding capture sequence may be introduced to, or otherwise induced in, a biological particle (e.g., cell, cell bead, etc.) for performing different assays in various formats (e.g., barcoded antibodies comprising the corresponding capture sequence, barcoded MHC dextramers comprising the corresponding capture sequence, barcoded guide RNA molecules comprising the corresponding capture sequence, etc.), such that the corresponding capture sequence may later interact with the capture sequence associated with the bead. In some instances, a capture sequence coupled to a bead (or other support) may be configured to attach to a linker molecule, such as a splint molecule, wherein the linker molecule is configured to couple the bead (or other support) to other molecules through the linker molecule, such as to one or more analytes or one or more other linker molecules.

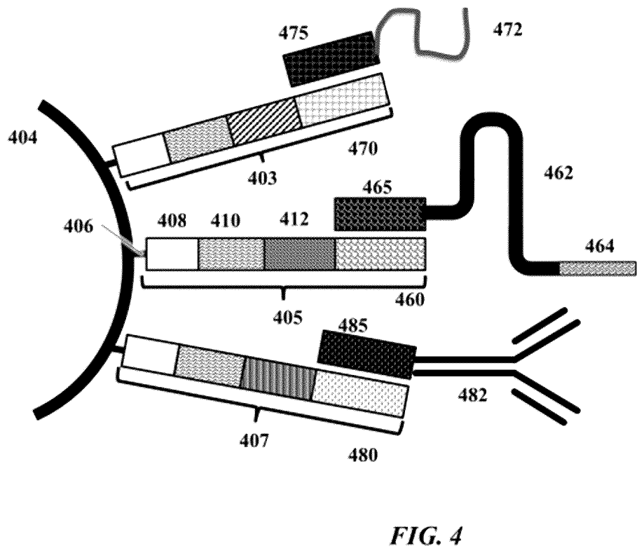

FIG. 4 illustrates another example of a barcode carrying bead. A nucleic acid barcode molecule 405, such as an oligonucleotide, can be coupled to a bead 404 by a releasable linkage 406, such as, for example, a disulfide linker. The nucleic acid barcode molecule 405 may comprise a first capture sequence 460. The same bead 404 may be coupled (e.g., via releasable linkage) to one or more other nucleic acid barcode molecules 403, 407 comprising other capture sequences. The nucleic acid barcode molecule 405 may be or comprise a barcode. As noted elsewhere herein, the structure of the barcode may comprise a number of sequence elements, such as a functional sequence 408 (e.g., flow cell attachment sequence, sequencing primer sequence, etc.), a barcode sequence 410 (e.g., bead-specific sequence common to bead, partition-specific sequence common to partition, etc.), and a unique molecular identifier 412 (e.g., unique sequence within different molecules attached to the bead), or partial sequences thereof. The capture sequence 460 may be configured to attach to a corresponding capture sequence 465. In some instances, the corresponding capture sequence 465 may be coupled to another molecule that may be an analyte or an intermediary carrier. For example, as illustrated in FIG. 4, the corresponding capture sequence 465 is coupled to a guide RNA molecule 462 comprising a target sequence 464, wherein the target sequence 464 is configured to attach to the analyte. Another oligonucleotide molecule 407 attached to the bead 404 comprises a second capture sequence 480 which is configured to attach to a second corresponding capture sequence 485. As illustrated in FIG. 4, the second corresponding capture sequence 485 is coupled to an antibody 482. In some cases, the antibody 482 may have binding specificity to an analyte (e.g., surface protein). Alternatively, the antibody 482 may not have binding specificity. Another oligonucleotide molecule 403 attached to the bead 404 comprises a third capture sequence 470 which is configured to attach to a second corresponding capture sequence 475. As illustrated in FIG. 4, the third corresponding capture sequence 475 is coupled to a molecule 472. The molecule 472 may or may not be configured to target an analyte. The other oligonucleotide molecules 403, 407 may comprise the other sequences (e.g., functional sequence, barcode sequence, UMI, etc.) described with respect to oligonucleotide molecule 405. While a single oligonucleotide molecule comprising each capture sequence is illustrated in 4, it will be appreciated that, for each capture sequence, the bead may comprise a set of one or more oligonucleotide molecules each comprising the capture sequence. For example, the bead may comprise any number of sets of one or more different capture sequences. Alternatively or in addition, the bead 404 may comprise other capture sequences. Alternatively or in addition, the bead 404 may comprise fewer types of capture sequences (e.g., two capture sequences). Alternatively or in addition, the bead 404 may comprise oligonucleotide molecule(s) comprising a priming sequence, such as a specific priming sequence such as an mRNA specific priming sequence (e.g., poly-T sequence), a targeted priming sequence, and/or a random priming sequence, for example, to facilitate an assay for gene expression.

In operation, the barcoded oligonucleotides may be released (e.g., in a partition), as described elsewhere herein. Alternatively, the nucleic acid barcode molecules bound to the bead (e.g., gel bead) may be used to hybridize and capture analytes (e.g., one or more types of analytes) on the solid phase of the bead.

In some cases, precursors comprising a functional group that is reactive or capable of being activated such that it becomes reactive can be polymerized with other precursors to generate gel beads comprising the activated or activatable functional group. The functional group may then be used to attach additional species (e.g., disulfide linkers, primers, other oligonucleotides, etc.) to the gel beads. For example, some precursors comprising a carboxylic acid (COOH) group can co-polymerize with other precursors to form a gel bead that also comprises a COOH functional group. In some cases, acrylic acid (a species comprising free COOH groups), acrylamide, and bis(acryloyl)cystamine can be co-polymerized together to generate a gel bead comprising free COOH groups. The COOH groups of the gel bead can be activated (e.g., via 1-Ethyl-3-(3-dimethylaminopropyl)carbodiimide (EDC) and N-Hydroxysuccinimide (NHS) or 4-(4,6-Dimethoxy-1,3,5-triazin-2-yl)-4-methylmorpholinium chloride (DMTMM)) such that they are reactive (e.g., reactive to amine functional groups where EDC/NHS or DMTMM are used for activation). The activated COOH groups can then react with an appropriate species (e.g., a species comprising an amine functional group where the carboxylic acid groups are activated to be reactive with an amine functional group) comprising a moiety to be linked to the bead.

Beads comprising disulfide linkages in their polymeric network may be functionalized with additional species via reduction of some of the disulfide linkages to free thiols. The disulfide linkages may be reduced via, for example, the action of a reducing agent (e.g., DTT, TCEP, etc.) to generate free thiol groups, without dissolution of the bead. Free thiols of the beads can then react with free thiols of a species or a species comprising another disulfide bond (e.g., via thiol-disulfide exchange) such that the species can be linked to the beads (e.g., via a generated disulfide bond). In some cases, free thiols of the beads may react with any other suitable group. For example, free thiols of the beads may react with species comprising an acrydite moiety. The free thiol groups of the beads can react with the acrydite via Michael addition chemistry, such that the species comprising the acrydite is linked to the bead. In some cases, uncontrolled reactions can be prevented by inclusion of a thiol capping agent such as N-ethylmalieamide or iodoacetate.

Activation of disulfide linkages within a bead can be controlled such that only a small number of disulfide linkages are activated. Control may be exerted, for example, by controlling the concentration of a reducing agent used to generate free thiol groups and/or concentration of reagents used to form disulfide bonds in bead polymerization. In some cases, a low concentration (e.g., molecules of reducing agent:gel bead ratios of less than or equal to about 1:100,000,000,000, less than or equal to about 1:10,000,000,000, less than or equal to about 1:1,000,000,000, less than or equal to about 1:100,000,000, less than or equal to about 1:10,000,000, less than or equal to about 1:1,000,000, less than or equal to about 1:100,000, less than or equal to about 1:10,000) of reducing agent may be used for reduction. Controlling the number of disulfide linkages that are reduced to free thiols may be useful in ensuring bead structural integrity during functionalization. In some cases, optically-active agents, such as fluorescent dyes may be coupled to beads via free thiol groups of the beads and used to quantify the number of free thiols present in a bead and/or track a bead.

In some cases, addition of moieties to a gel bead after gel bead formation may be advantageous. For example, addition of an oligonucleotide (e.g., barcoded oligonucleotide) after gel bead formation may avoid loss of the species during chain transfer termination that can occur during polymerization. Moreover, smaller precursors (e.g., monomers or cross linkers that do not comprise side chain groups and linked moieties) may be used for polymerization and can be minimally hindered from growing chain ends due to viscous effects. In some cases, functionalization after gel bead synthesis can minimize exposure of species (e.g., oligonucleotides) to be loaded with potentially damaging agents (e.g., free radicals) and/or chemical environments. In some cases, the generated gel may possess an upper critical solution temperature (UCST) that can permit temperature driven swelling and collapse of a bead. Such functionality may aid in oligonucleotide (e.g., a primer) infiltration into the bead during subsequent functionalization of the bead with the oligonucleotide. Post-production functionalization may also be useful in controlling loading ratios of species in beads, such that, for example, the variability in loading ratio is minimized. Species loading may also be performed in a batch process such that a plurality of beads can be functionalized with the species in a single batch.

A bead injected or otherwise introduced into a partition may comprise releasably, cleavably, or reversibly attached barcodes. A bead injected or otherwise introduced into a partition may comprise activatable barcodes. A bead injected or otherwise introduced into a partition may be degradable, disruptable, or dissolvable beads.

Barcodes can be releasably, cleavably or reversibly attached to the beads such that barcodes can be released or be releasable through cleavage of a linkage between the barcode molecule and the bead, or released through degradation of the underlying bead itself, allowing the barcodes to be accessed or be accessible by other reagents, or both. In non-limiting examples, cleavage may be achieved through reduction of di-sulfide bonds, use of restriction enzymes, photo-activated cleavage, or cleavage via other types of stimuli (e.g., chemical, thermal, pH, enzymatic, etc.) and/or reactions, such as described elsewhere herein. Releasable barcodes may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

In addition to, or as an alternative to the cleavable linkages between the beads and the associated molecules, such as barcode containing nucleic acid barcode molecules (e.g., barcoded oligonucleotides), the beads may be degradable, disruptable, or dissolvable spontaneously or upon exposure to one or more stimuli (e.g., temperature changes, pH changes, exposure to particular chemical species or phase, exposure to light, reducing agent, etc.). In some cases, a bead may be dissolvable, such that material components of the beads are solubilized when exposed to a particular chemical species or an environmental change, such as a change temperature or a change in pH. In some cases, a gel bead can be degraded or dissolved at elevated temperature and/or in basic conditions. In some cases, a bead may be thermally degradable such that when the bead is exposed to an appropriate change in temperature (e.g., heat), the bead degrades. Degradation or dissolution of a bead bound to a species (e.g., a nucleic acid barcode molecule, e.g., barcoded oligonucleotide) may result in release of the species from the bead.

As will be appreciated from the above disclosure, the degradation of a bead may refer to the disassociation of a bound or entrained species from a bead, both with and without structurally degrading the physical bead itself. For example, the degradation of the bead may involve cleavage of a cleavable linkage via one or more species and/or methods described elsewhere herein. In another example, entrained species may be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead may cause a bead to better retain an entrained species due to pore size contraction.

A degradable bead may be introduced into a partition, such as a droplet of an emulsion or a well, such that the bead degrades within the partition and any associated species (e.g., oligonucleotides) are released within the droplet when the appropriate stimulus is applied. The free species (e.g., oligonucleotides, nucleic acid barcode molecules) may interact with other reagents contained in the partition. For example, a polyacrylamide bead comprising cystamine and linked, via a disulfide bond, to a barcode sequence, may be combined with a reducing agent within a droplet of a water-in-oil emulsion. Within the droplet, the reducing agent can break the various disulfide bonds, resulting in bead degradation and release of the barcode sequence into the aqueous, inner environment of the droplet. In another example, heating of a droplet comprising a bead-bound barcode sequence in basic solution may also result in bead degradation and release of the attached barcode sequence into the aqueous, inner environment of the droplet.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing nucleic acid molecule (e.g., oligonucleotide) bearing beads.

In some cases, beads can be non-covalently loaded with one or more reagents. The beads can be non-covalently loaded by, for instance, subjecting the beads to conditions sufficient to swell the beads, allowing sufficient time for the reagents to diffuse into the interiors of the beads, and subjecting the beads to conditions sufficient to de-swell the beads. The swelling of the beads may be accomplished, for instance, by placing the beads in a thermodynamically favorable solvent, subjecting the beads to a higher or lower temperature, subjecting the beads to a higher or lower ion concentration, and/or subjecting the beads to an electric field. The swelling of the beads may be accomplished by various swelling methods. The de-swelling of the beads may be accomplished, for instance, by transferring the beads in a thermodynamically unfavorable solvent, subjecting the beads to lower or high temperatures, subjecting the beads to a lower or higher ion concentration, and/or removing an electric field. The de-swelling of the beads may be accomplished by various de-swelling methods. Transferring the beads may cause pores in the bead to shrink. The shrinking may then hinder reagents within the beads from diffusing out of the interiors of the beads. The hindrance may be due to steric interactions between the reagents and the interiors of the beads. The transfer may be accomplished microfluidically. For instance, the transfer may be achieved by moving the beads from one co-flowing solvent stream to a different co-flowing solvent stream. The swellability and/or pore size of the beads may be adjusted by changing the polymer composition of the bead.

In some cases, an acrydite moiety linked to a precursor, another species linked to a precursor, or a precursor itself can comprise a labile bond, such as chemically, thermally, or photo-sensitive bond e.g., disulfide bond, UV sensitive bond, or the like. Once acrydite moieties or other moieties comprising a labile bond are incorporated into a bead, the bead may also comprise the labile bond. The labile bond may be, for example, useful in reversibly linking (e.g., covalently linking) species (e.g., barcodes, primers, etc.) to a bead. In some cases, a thermally labile bond may include a nucleic acid hybridization based attachment, e.g., where an oligonucleotide is hybridized to a complementary sequence that is attached to the bead, such that thermal melting of the hybrid releases the oligonucleotide, e.g., a barcode containing sequence, from the bead or microcapsule.

The addition of multiple types of labile bonds to a gel bead may result in the generation of a bead capable of responding to varied stimuli. Each type of labile bond may be sensitive to an associated stimulus (e.g., chemical stimulus, light, temperature, enzymatic, etc.) such that release of species attached to a bead via each labile bond may be controlled by the application of the appropriate stimulus. Such functionality may be useful in controlled release of species from a gel bead. In some cases, another species comprising a labile bond may be linked to a gel bead after gel bead formation via, for example, an activated functional group of the gel bead as described above. As will be appreciated, barcodes that are releasably, cleavably or reversibly attached to the beads described herein include barcodes that are released or releasable through cleavage of a linkage between the barcode molecule and the bead, or that are released through degradation of the underlying bead itself, allowing the barcodes to be accessed or accessible by other reagents, or both.

In some cases, a species (e.g., oligonucleotide molecules comprising barcodes) that are attached to a solid support (e.g., a bead) may comprise a U-excising element that allows the species to release from the bead. In some cases, the U-excising element may comprise a single-stranded DNA (ssDNA) sequence that contains at least one uracil. The species may be attached to a solid support via the ssDNA sequence containing the at least one uracil. The species may be released by a combination of uracil-DNA glycosylase (e.g., to remove the uracil) and an endonuclease (e.g., to induce an ssDNA break). If the endonuclease generates a 5′ phosphate group from the cleavage, then additional enzyme treatment may be included in downstream processing to eliminate the phosphate group, e.g., prior to ligation of additional sequencing handle elements, e.g., Illumina full P5 sequence, partial P5 sequence, full R1 sequence, and/or partial R1 sequence.

The barcodes that are releasable as described herein may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

In addition to thermally cleavable bonds, disulfide bonds and UV sensitive bonds, other non-limiting examples of labile bonds that may be coupled to a precursor or bead include an ester linkage (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNAase)). A bond may be cleavable via other nucleic acid molecule targeting enzymes, such as restriction enzymes (e.g., restriction endonucleases), as described further below.

Species may be encapsulated in beads during bead generation (e.g., during polymerization of precursors). Such species may or may not participate in polymerization. Such species may be entered into polymerization reaction mixtures such that generated beads comprise the species upon bead formation. In some cases, such species may be added to the gel beads after formation. Such species may include, for example, nucleic acid barcode molecules (e.g., oligonucleotides), reagents for a nucleic acid amplification reaction (e.g., primers, polymerases, dNTPs, co-factors (e.g., ionic co-factors), buffers) including those described herein, reagents for enzymatic reactions (e.g., enzymes, co-factors, substrates, buffers), reagents for nucleic acid modification reactions such as polymerization, ligation, or digestion, and/or reagents for template preparation (e.g., tagmentation) for one or more sequencing platforms (e.g., Nextera® for Illumina®). Such species may include one or more enzymes described herein, including without limitation, polymerase, reverse transcriptase, restriction enzymes (e.g., endonuclease), transposase, ligase, proteinase K, DNAse, etc. Such species may include one or more reagents described elsewhere herein (e.g., lysis agents, inhibitors, inactivating agents, chelating agents, stimulus). Trapping of such species may be controlled by the polymer network density generated during polymerization of precursors, control of ionic charge within the gel bead (e.g., via ionic species linked to polymerized species), or by the release of other species. Encapsulated species may be released from a bead upon bead degradation and/or by application of a stimulus capable of releasing the species from the bead. Alternatively or in addition, species may be partitioned in a partition (e.g., droplet) during or subsequent to partition formation. Such species may include, without limitation, the abovementioned species that may also be encapsulated in a bead.

A degradable bead may comprise one or more species with a labile bond such that, when the bead/species is exposed to the appropriate stimuli, the bond is broken and the bead degrades. The labile bond may be a chemical bond (e.g., covalent bond, ionic bond) or may be another type of physical interaction (e.g., van der Waals interactions, dipole-dipole interactions, etc.). In some cases, a crosslinker used to generate a bead may comprise a labile bond. Upon exposure to the appropriate conditions, the labile bond can be broken and the bead degraded. For example, upon exposure of a polyacrylamide gel bead comprising cystamine crosslinkers to a reducing agent, the disulfide bonds of the cystamine can be broken and the bead degraded.

A degradable bead may be useful in more quickly releasing an attached species (e.g., a nucleic acid molecule, a barcode sequence, a primer, etc.) from the bead when the appropriate stimulus is applied to the bead as compared to a bead that does not degrade. For example, for a species bound to an inner surface of a porous bead or in the case of an encapsulated species, the species may have greater mobility and accessibility to other species in solution upon degradation of the bead. In some cases, a species may also be attached to a degradable bead via a degradable linker (e.g., disulfide linker). The degradable linker may respond to the same stimuli as the degradable bead or the two degradable species may respond to different stimuli. For example, a barcode sequence may be attached, via a disulfide bond, to a polyacrylamide bead comprising cystamine. Upon exposure of the barcoded-bead to a reducing agent, the bead degrades and the barcode sequence is released upon breakage of both the disulfide linkage between the barcode sequence and the bead and the disulfide linkages of the cystamine in the bead.

As will be appreciated from the above disclosure, while referred to as degradation of a bead, in many instances as noted above, that degradation may refer to the disassociation of a bound or entrained species from a bead, both with and without structurally degrading the physical bead itself. For example, entrained species may be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead may cause a bead to better retain an entrained species due to pore size contraction.

Where degradable beads are provided, it may be beneficial to avoid exposing such beads to the stimulus or stimuli that cause such degradation prior to a given time, in order to, for example, avoid premature bead degradation and issues that arise from such degradation, including for example poor flow characteristics and aggregation. By way of example, where beads comprise reducible cross-linking groups, such as disulfide groups, contacting such beads with reducing agents, e.g., DTT or other disulfide cleaving reagents can be avoided. In such cases, treatment to the beads described herein will, in some cases be provided free of reducing agents, such as DTT. Because reducing agents are often provided in commercial enzyme preparations, reducing agent free (or DTT free) enzyme preparations can be provided in treating the beads described herein. Examples of such enzymes include, e.g., polymerase enzyme preparations, reverse transcriptase enzyme preparations, ligase enzyme preparations, as well as many other enzyme preparations that may be used to treat the beads described herein. The terms “reducing agent free” or “DTT free” preparations can refer to a preparation having less than about 1/10, less than about 1/50, or even less than about 1/100 of the lower ranges for such materials used in degrading the beads. For example, for DTT, the reducing agent free preparation can have less than about 0.01 millimolar (mM), 0.005 mM, 0.001 mM DTT, 0.0005 mM DTT, or even less than about 0.0001 mM DTT. In many cases, the amount of DTT can be undetectable.

Numerous chemical triggers may be used to trigger the degradation of beads. Examples of these chemical changes may comprise pH-mediated changes to the integrity of a component within the bead, degradation of a component of a bead via cleavage of cross-linked bonds, and depolymerization of a component of a bead.

In some cases, a bead may be formed from materials that comprise degradable chemical crosslinkers, such as BAC or cystamine. Degradation of such degradable crosslinkers may be accomplished through a number of mechanisms. In some examples, a bead may be contacted with a chemical degrading agent that may induce oxidation, reduction or other chemical changes. For example, a chemical degrading agent may be a reducing agent, such as dithiothreitol (DTT). Additional examples of reducing agents may include ß-mercaptoethanol, (2S)-2-amino-1,4-dimercaptobutane (dithiobutylamine or DTBA), tris(2-carboxyethyl) phosphine (TCEP), or combinations thereof. A reducing agent may degrade the disulfide bonds formed between gel precursors forming the bead, and thus, degrade the bead. In other cases, a change in pH of a solution, such as an increase in pH, may trigger degradation of a bead. In other cases, exposure to an aqueous solution, such as water, may trigger hydrolytic degradation, and thus degradation of the bead. In some cases, any combination of stimuli may trigger degradation of a bead. For example, a change in pH may enable a chemical agent (e.g., DTT) to become an effective reducing agent.

Beads may also be induced to release their contents upon the application of a thermal stimulus. A change in temperature can cause a variety of changes to a bead. For example, heat can cause a solid bead to liquefy. A change in heat may cause melting of a bead such that a portion of the bead degrades. In other cases, heat may increase the internal pressure of the bead components such that the bead ruptures or explodes. Heat may also act upon heat-sensitive polymers used as materials to construct beads.

Any suitable agent may degrade beads. In some instances, changes in temperature or pH may be used to degrade thermo-sensitive or pH-sensitive bonds within beads. In some instances, chemical degrading agents may be used to degrade chemical bonds within beads by oxidation, reduction or other chemical changes. For example, a chemical degrading agent may be a reducing agent, such as DTT, wherein DTT may degrade the disulfide bonds formed between a crosslinker and gel precursors, thus degrading the bead. In some cases, a reducing agent may be added to degrade the bead, which may or may not cause the bead to release its contents. Examples of reducing agents may include dithiothreitol (DTT), ß-mercaptoethanol, (2S)-2-amino-1,4-dimercaptobutane (dithiobutylamine or DTBA), tris(2-carboxyethyl) phosphine (TCEP), or combinations thereof. The reducing agent may be present at a concentration of about 0.1 mM, 0.5 mM, 1 mM, 5 mM, 10 mM. The reducing agent may be present at a concentration of at least about 0.1 mM, 0.5 mM, 1 mM, 5 mM, 10 mM, or greater than 10 mM. The reducing agent may be present at concentration of at most about 10 mM, 5 mM, 1 mM, 0.5 mM, 0.1 mM, or less.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing oligonucleotide bearing beads.

In some examples, a partition of the plurality of partitions may comprise a single biological particle or analyte carrier (e.g., a single cell or a single nucleus of a cell). In some examples, a partition of the plurality of partitions may comprise multiple biological particles or analyte carriers. Such partitions may be referred to as multiply occupied partitions, and may comprise, for example, two, three, four or more cells and/or microcapsules (e.g., beads) comprising barcoded nucleic acid molecules (e.g., oligonucleotides) within a single partition. Accordingly, as noted above, the flow characteristics of the biological particle and/or bead containing fluids and partitioning fluids may be controlled to provide for such multiply occupied partitions. In particular, the flow parameters may be controlled to provide a given occupancy rate at greater than about 50% of the partitions, greater than about 75%, and in some cases greater than about 80%, 90%, 95%, or higher.

In some cases, additional microcapsules can be used to deliver additional reagents to a partition. In such cases, it may be advantageous to introduce different beads into a common channel or droplet generation junction, from different bead sources (e.g., containing different associated reagents) through different channel inlets into such common channel or droplet generation junction. In such cases, the flow and frequency of the different beads into the channel or junction may be controlled to provide for a certain ratio of microcapsules from each source, while ensuring a given pairing or combination of such beads into a partition with a given number of biological particles (e.g., one biological particle and one bead per partition).

The partitions described herein may comprise small volumes, for example, less than about 10 microliters (μL), 5 μL, 1 μL, 900 picoliters (pL), 800 pL, 700 pL, 600 pL, 500 pL, 400 pL, 300 pL, 200 pL, 100 pL, 50 pL, 20 pL, 10 pL, 1 pL, 500 nanoliters (nL), 100 nL, 50 nL, or less.

For example, in the case of droplet based partitions, the droplets may have overall volumes that are less than about 1000 pL, 900 pL, 800 pL, 700 pL, 600 pL, 500 pL, 400 pL, 300 pL, 200 pL, 100 pL, 50 pL, 20 pL, 10 pL, 1 pL, or less. Where co-partitioned with microcapsules, it will be appreciated that the sample fluid volume, e.g., including co-partitioned biological particles and/or beads, within the partitions may be less than about 90% of the above described volumes, less than about 80%, less than about 70%, less than about 60%, less than about 50%, less than about 40%, less than about 30%, less than about 20%, or less than about 10% of the above described volumes.

As is described elsewhere herein, partitioning species may generate a population or plurality of partitions. In such cases, any suitable number of partitions can be generated or otherwise provided. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided. Moreover, the plurality of partitions may comprise both unoccupied partitions (e.g., empty partitions) and occupied partitions.

### Flow Sorting

A sample may derive from any useful source including any subject, such as a human subject. A sample may comprise material (e.g., one or more analyte carriers) from one or more different sources, such as one or more different subjects. Multiple samples, such as multiple samples from a single subject (e.g., multiple samples obtained in the same or different manners from the same or different bodily locations, and/or obtained at the same or different times (e.g., seconds, minutes, hours, days, weeks, months, or years apparat)), or multiple samples from different subjects, may be obtained for analysis as described herein. For example, a first sample may be obtained from a subject at a first time and a second sample may be obtained from the subject at a second time later than the first time. The first time may be before a subject undergoes a treatment regimen or procedure (e.g., to address a disease or condition), and the second time may be during or after the subject undergoes the treatment regimen or procedure. In another example, a first sample may be obtained from a first bodily location or system of a subject (e.g., using a first collection technique) and a second sample may be obtained from a second bodily location or system of the subject (e.g., using a second collection technique), which second bodily location or system may be different than the first bodily location or system. In another example, multiple samples may be obtained from a subject at a same time from the same or different bodily locations. Different samples, such as different subjects collected from different bodily locations of a same subject, at different times, from multiple different subjects, and/or using different collection techniques, may undergo the same or different processing (e.g., as described herein). For example, a first sample may undergo a first processing protocol and a second sample may undergo a second processing protocol.

A sample may be a biological sample, such as a cell sample (e.g., as described herein). A sample may include one or more analyte carriers, such as one or more cells and/or cellular constituents, such as one or more cell nuclei. For example, a sample may comprise a plurality of analyte carriers, such as a plurality of cells and/or cellular constituents. Analyte carriers (e.g., cells or cellular constituents, such as cell nuclei) of a sample may be of a single type or a plurality of different types. For example, cells of a sample may include one or more different types or blood cells.

Cells and cellular constituents of a sample may be of any type. For example, a cell or cellular constituent may be a mammalian, fungal, plant, bacterial, or other cell type. In some cases, the cell is a mammalian cell, such as a human cell. The cell may be, for example, a stem cell, liver cell, nerve cell, bone cell, blood cell, reproductive cell, skin cell, skeletal muscle cell, cardiac muscle cell, smooth muscle cell, hair cell, hormone-secreting cell, or glandular cell. The cell may be, for example, an erythrocyte (e.g., red blood cell), a megakaryocyte (e.g., platelet precursor), a monocyte (e.g., white blood cell), a leukocyte, a B cell, a T cell (such as a helper, suppressor, cytotoxic, or natural killer T cell), an osteoclast, a dendritic cell, a connective tissue macrophage, an epidermal Langerhans cell, a microglial cell, a granulocyte, a hybridoma cell, a mast cell, a natural killer cell, a reticulocyte, a hematopoietic stem cell, a myoepithelial cell, a myeloid-derived suppressor cell, a platelet, a thymocyte, a satellite cell, an epithelial cell, an endothelial cell, an epididymal cell, a kidney cell, a liver cell, an adipocyte, a lipocyte, or a neuron cell. In some cases, the cell may be associated with a cancer, tumor, or neoplasm. In some cases, the cell may be associated with a fetus. In some cases, the cell may be a Jurkat cell.

A cell of a biological sample may have any feature or dimension. For example, a cell may have a first dimension, a second dimension, and a third dimension, where the first, second, and third dimensions are approximately the same. In other cases, the first and second dimensions may be approximately the same, and the third dimension may be different, or the first, second, and third dimensions may all be different. In some cases, a cell may comprise a dimension (e.g., a diameter) of at least about 1 μm. For example, a cell may comprise a dimension of at least about 1 micrometer (μm), 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 100 μm, 120 μm, 140 μm, 160 μm, 180 μm, 200 μm, 250 μm, 300 μm, 350 μm, 400 μm, 450 μm, 500 μm, 550 μm, 600 μm, 650 μm, 700 μm, 750 μm, 800 μm, 850 μm, 900 μm, 950 μm, 1 millimeter (mm), or greater. In some cases, the cell may comprise a dimension of between about 1 μm and 500 μm, such as between about 1 μm and 100 μm, between about 100 μm and 200 μm, between about 200 μm and 300 μm, between about 300 μm and 400 μm, or between about 400 μm and 500 μm. For example, a cell may comprise a dimension of between about 1 μm and 100 μm. Any or all dimensions of a cell may be variable. For example, the dimensions of a substantially fluid cell may vary over a rapid timescale. Dimensions of a more rigid cell may be fixed or may vary with lesser amplitude. Accordingly, the dimensions provided herein may represent averages rather than fixed values. The volume of a cell may be at least about 1 μm³. In some cases, the volume of a cell may be at least about 10 μm³. For example, the volume of the cell may be at least 1 μm³, 2 μm³, 3 μm³, 4 μm³, 5 μm³, 6 μm³, 7 μm³, 8 μm³, 9 μm³, 10 μm³, 12 μm³, 14 μm³, 16 μm³, 18 μm³, 20 μm³, 25 μm³, 30 μm³, 35 μm³, 40 μm³, 45 μm³, 50 μm³, 55 μm³, 60 μm³, 65 μm³, 70 μm³, 75 μm³, 80 μm³, 85 μm³, 90 μm³, 95 μm³, 100 μm³, 125 μm³, 150 μm³, 175 μm³, 200 μm³, 250 μm³, 300 μm³, 350 μm³, 400 μm³, 450 μm³, μm³, 500 μm³, 550 μm³, 600 μm³, 650 μm³, 700 μm³, 750 μm³, 800 μm³, 850 μm³, 900 μm³, 950 μm³, 1000 μm³, 1200 μm³, 1400 μm³, 1600 μm³, 1800 μm³, 2000 μm³, 2200 μm³, 2400 μm³, 2600 μm³, 2800 μm³, 3000 μm³, or greater. In some cases, a cell may comprise a volume of between about 1 μm³ and 100 μm³, such as between about 1 μm³ and 10 μm³, between about 10 μm³ and 50 μm³, or between about 50 μm³ and 100 μm³. In some cases, a cell may comprise a volume of between about 100 μm³ and 1000 μm³, such as between about 100 μm³ and 500 μm³ or between about 500 μm³ and 1000 μm³. In some cases, a cell may comprise a volume between about 1000 μm³ and 3000 μm³, such as between about 1000 μm³ and 2000 μm³ or between about 2000 μm³ and 3000 μm³. In some cases, a cell may comprise a volume between about 1 μm³ and 3000 μm³, such as between about 1 μm³ and 2000 μm³, between about 1 μm³ and 1000 μm³, between about 1 μm³ and 500 μm³, or between about 1 μm³ and 250 μm³.

A cell of a biological sample may comprise one or more cross-sections that may be the same or different. In some cases, a cell may have a first cross-section that is different from a second cross-section. a cell may have a first cross-section that is at least about 1 μm. For example, a cell may comprise a cross-section (e.g., a first cross-section) of at least about 1 micrometer (μm), 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 100 μm, 120 μm, 140 μm, 160 μm, 180 μm, 200 μm, 250 μm, 300 μm, 350 μm, 400 μm, 450 μm, 500 μm, 550 μm, 600 μm, 650 μm, 700 μm, 750 μm, 800 μm, 850 μm, 900 μm, 950 μm, 1 millimeter (mm), or greater. In some cases, a cell may comprise a cross-section (e.g., a first cross-section) of between about 1 μm and 500 μm, such as between about 1 μm and 100 μm, between about 100 μm and 200 μm, between about 200 μm and 300 μm, between about 300 μm and 400 μm, or between about 400 μm and 500 μm. For example, a cell may comprise a cross-section (e.g., a first cross-section) of between about 1 μm and 100 μm. In some cases, the cell may have a second cross-section that is at least about 1 μm. For example, the cell may comprise a second cross-section of at least about 1 micrometer (μm), 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 100 μm, 120 μm, 140 μm, 160 μm, 180 μm, 200 μm, 250 μm, 300 μm, 350 μm, 400 μm, 450 μm, 500 μm, 550 μm, 600 μm, 650 μm, 700 μm, 750 μm, 800 μm, 850 μm, 900 μm, 950 μm, 1 millimeter (mm), or greater. In some cases, a cell may comprise a second cross-section of between about 1 μm and 500 μm, such as between about 1 μm and 100 μm, between about 100 μm and 200 μm, between about 200 μm and 300 μm, between about 300 μm and 400 μm, or between about 400 μm and 500 μm. For example, a cell may comprise a second cross-section of between about 1 μm and 100 μm.

A cross section (e.g., a first cross-section) may correspond to a diameter of a cell. In some cases, a cell may be approximately spherical. In such cases, the first cross-section may correspond to the diameter of the cell. In other cases, the cell may be approximately cylindrical. In such cases, the first cross-section may correspond to a diameter, length, or width along the approximately cylindrical cell. In some cases, the cell may comprise a surface. A cell surface may comprise one or more features. For example, a cell may comprise a dendritic receiver, flagella, roughed border, or other feature.

A characteristic or set of characteristics of a cell may be changed by one or more conditions. A condition suitable for changing a characteristic or set of characteristics of a cell may be, for example, a temperature, a pH, an ion or salt concentration, a pressure, or another condition. For example, a cell may be exposed to a chemical species that may bring about a change in one or more characteristics of the cell. In some cases, a stimulus may be used to change one or more characteristics of a cell. For example, upon application of the stimulus, one or more characteristics of a cell may be changed. The stimulus may be, for example, a thermal stimulus, a photo stimulus, a chemical stimulus, or another stimulus. In some cases, conditions sufficient to change the one or more characteristics of a cell may comprise one or more different conditions, such as a temperature and a pressure, a pH and a salt concentration, a chemical species and a temperature, or any other combination of conditions. A temperature sufficient for changing one or more characteristics of the cell may be, for example, at least about 0 degrees Celsius (C), 1° C., 2° C., 3° C., 4° C., 5° C., 10° C., or higher. For example, the temperature may be about 4° C. In other cases, a temperature sufficient for changing one or more characteristics of the cell may be, for example, at least about 25° C., 30° C., 35° C., 37° C., 40° C., 45° C., 50° C., or higher. For example, the temperature may be about 37° C. A pH sufficient for changing one or more characteristics of the cell may be, for example, between about 5 and 8, such as between about 6 and 7.

A biological sample may include a plurality of cells having different dimensions and features. In some cases, processing of the biological sample, such as cell separation and sorting (e.g., as described herein), may affect the distribution of dimensions and cellular features included in the sample by depleting cells having certain features and dimensions and/or isolating cells having certain features and dimensions.

A sample may undergo one or more processes in preparation for analysis (e.g., as described herein), including, but not limited to, filtration, selective precipitation, purification, centrifugation, permeabilization, isolation, agitation, heating, and/or other processes. For example, a sample may be filtered to remove a contaminant or other materials. In an example, a filtration process may comprise the use of microfluidics (e.g., to separate analyte carriers of different sizes, types, charges, or other features).

In an example, a sample comprising one or more cells may be processed to separate the one or more cells from other materials in the sample (e.g., using centrifugation and/or another process). In some cases, cells and/or cellular constituents of a sample may be processed to separate and/or sort groups of cells and/or cellular constituents, such as to separate and/or sort cells and/or cellular constituents of different types. Examples of cell separation include, but are not limited to, separation of white blood cells or immune cells from other blood cells and components, separation of circulating tumor cells from blood, and separation of bacteria from bodily cells and/or environmental materials. A separation process may comprise a positive selection process (e.g., targeting of a cell type of interest for retention for subsequent downstream analysis, such as by use of a monoclonal antibody that targets a surface marker of the cell type of interest), a negative selection process (e.g., removal of one or more cell types and retention of one or more other cell types of interest), and/or a depletion process (e.g., removal of a single cell type from a sample, such as removal of red blood cells from peripheral blood mononuclear cells). Separation of one or more different types of cells may comprise, for example, centrifugation, filtration, microfluidic-based sorting, flow cytometry, fluorescence-activated cell sorting (FACS), magnetic-activated cell sorting (MACS), buoyancy-activated cell sorting (BACS), or any other useful method. For example, a flow cytometry method may be used to detect cells and/or cellular constituents based on a parameter such as a size, morphology, or protein expression. Flow cytometry-based cell sorting may comprise injecting a sample into a sheath fluid that conveys the cells and/or cellular constituents of the sample into a measurement region one at a time. In the measurement region, a light source such as a laser may interrogate the cells and/or cellular constituents and scattered light and/or fluorescence may be detected and converted into digital signals. A nozzle system (e.g., a vibrating nozzle system) may be used to generate droplets (e.g., aqueous droplets) comprising individual cells and/or cellular constituents. Droplets including cells and/or cellular constituents of interest (e.g., as determined via optical detection) may be labeled with an electric charge (e.g., using an electrical charging ring), which charge may be used to separate such droplets from droplets including other cells and/or cellular constituents. For example, FACS may comprise labeling cells and/or cellular constituents with fluorescent markers (e.g., using internal and/or external biomarkers). Cells and/or cellular constituents may then be measured and identified one by one and sorted based on the emitted fluorescence of the marker or absence thereof. MACS may use micro- or nano-scale magnetic particles to bind to cells and/or cellular constituents (e.g., via an antibody interaction with cell surface markers) to facilitate magnetic isolation of cells and/or cellular constituents of interest from other components of a sample (e.g., using a column-based analysis). BACS may use microbubbles (e.g., glass microbubbles) labeled with antibodies to target cells of interest. Cells and/or cellular components coupled to microbubbles may float to a surface of a solution, thereby separating target cells and/or cellular components from other components of a sample. Cell separation techniques may be used to enrich for populations of cells of interest (e.g., prior to partitioning, as described herein). For example, a sample comprising a plurality of cells including a plurality of cells of a given type may be subjected to a positive separation process. The plurality of cells of the given type may be labeled with a fluorescent marker (e.g., based on an expressed cell surface marker or another marker) and subjected to a FACS process to separate these cells from other cells of the plurality of cells. The selected cells may then be subjected to subsequent partition-based analysis (e.g., as described herein) or other downstream analysis. The fluorescent marker may be removed prior to such analysis or may be retained. The fluorescent marker may comprise an identifying feature, such as a nucleic acid barcode sequence and/or unique molecular identifier.

In another example, a first sample comprising a first plurality of cells including a first plurality of cells of a given type (e.g., immune cells expressing a particular marker or combination of markers) and a second sample comprising a second plurality of cells including a second plurality of cells of the given type may be subjected to a positive separation process. The first and second samples may be collected from the same or different subjects, at the same or different types, from the same or different bodily locations or systems, using the same or different collection techniques. For example, the first sample may be from a first subject and the second sample may be from a second subject different than the first subject. The first plurality of cells of the first sample may be provided a first plurality of fluorescent markers configured to label the first plurality of cells of the given type. The second plurality of cells of the second sample may be provided a second plurality of fluorescent markers configured to label the second plurality of cells of the given type. The first plurality of fluorescent markers may include a first identifying feature, such as a first barcode, while the second plurality of fluorescent markers may include a second identifying feature, such as a second barcode, that is different than the first identifying feature. The first plurality of fluorescent markers and the second plurality of fluorescent markers may fluoresce at the same intensities and over the same range of wavelengths upon excitation with a same excitation source (e.g., light source, such as a laser). The first and second samples may then be combined and subjected to a FACS process to separate cells of the given type from other cells based on the first plurality of fluorescent markers labeling the first plurality of cells of the given type and the second plurality of fluorescent markers labeling the second plurality of cells of the given type. Alternatively, the first and second samples may undergo separate FACS processes and the positively selected cells of the given type from the first sample and the positively selected cells of the given type from the second sample may then be combined for subsequent analysis. The encoded identifying features of the different fluorescent markers may be used to identify cells originating from the first sample and cells originating from the second sample. For example, the first and second identifying features may be configured to interact (e.g., in partitions, as described herein) with nucleic acid barcode molecules (e.g., as described herein) to generate barcoded nucleic acid products detectable using, e.g., nucleic acid sequencing.

### Multiplexing

The present disclosure provides methods and systems for multiplexing, and otherwise increasing throughput in, analysis. For example, a single or integrated process workflow may permit the processing, identification, and/or analysis of more or multiple analytes, more or multiple types of analytes, and/or more or multiple types of analyte characterizations. For example, in the methods and systems described herein, one or more labelling agents capable of binding to or otherwise coupling to one or more cell features may be used to characterize analyte carriers and/or cell features. In some instances, cell features include cell surface features. Cell surface features may include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof. A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have a first reporter oligonucleotide coupled thereto, while a labelling agent that is specific to a different cell feature (e.g., a second cell surface feature) may have a different reporter oligonucleotide coupled thereto. For a description of example labelling agents, reporter oligonucleotides, and methods of use, see, e.g., U.S. Pat. No. 10,550,429; U.S. Pat. Pub. 20190177800; and U.S. 20190367969, each of which is herein entirely incorporated by reference for all purposes.

In a particular example, a library of potential cell feature labelling agents may be provided, where the respective cell feature labelling agents are associated with nucleic acid reporter molecules, such that a different reporter oligonucleotide sequence is associated with each labelling agent capable of binding to a specific cell feature. In some aspects, different members of the library may be characterized by the presence of a different oligonucleotide sequence label. For example, an antibody capable of binding to a first protein may have associated with it a first reporter oligonucleotide sequence, while an antibody capable of binding to a second protein may have a different reporter oligonucleotide sequence associated with it. The presence of the particular oligonucleotide sequence may be indicative of the presence of a particular antibody or cell feature which may be recognized or bound by the particular antibody.

Labelling agents capable of binding to or otherwise coupling to one or more analyte carriers may be used to characterize an analyte carrier as belonging to a particular set of analyte carriers. For example, labeling agents may be used to label a sample of cells or a group of cells. In this way, a group of cells may be labeled as different from another group of cells. In an example, a first group of cells may originate from a first sample and a second group of cells may originate from a second sample. Labelling agents may allow the first group and second group to have a different labeling agent (or reporter oligonucleotide associated with the labeling agent). This may, for example, facilitate multiplexing, where cells of the first group and cells of the second group may be labeled separately and then pooled together for downstream analysis. The downstream detection of a label may indicate analytes as belonging to a particular group.

For example, a reporter oligonucleotide may be linked to an antibody or an epitope binding fragment thereof, and labeling an analyte carrier may comprise subjecting the antibody-linked barcode molecule or the epitope binding fragment-linked barcode molecule to conditions suitable for binding the antibody to a molecule present on a surface of the analyte carrier. The binding affinity between the antibody or the epitope binding fragment thereof and the molecule present on the surface may be within a desired range to ensure that the antibody or the epitope binding fragment thereof remains bound to the molecule. For example, the binding affinity may be within a desired range to ensure that the antibody or the epitope binding fragment thereof remains bound to the molecule during various sample processing operations, such as partitioning and/or nucleic acid amplification or extension. A dissociation constant (Kd) between the antibody or an epitope binding fragment thereof and the molecule to which it binds may be less than about 100 μM. 90 μM, 80 μM, 70 μM, 60 μM, 50 μM, 40 μM, 30 μM, 20 PM, 10 μM, 9 μM, 8 μM, 7 μM, 6 μM, 5 μM, 4 μM, 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, 900 μM, 800 μM, 700 μM, 600 μM, 500 μM, 400 μM, 300 μM, 200 μM, 100 μM, 90 μM, 80 μM, 70 μM, 60 μM, 50 μM, 40 μM, 30 μM, 20 μM, 10 μM, 9 μM, 8 μM, 7 μM, 6 μM, 5 μM, 4 μM, 3 μM, 2 μM, or 1 μM For example, the dissociation constant may be less than about 10 μM.

In another example, a reporter oligonucleotide may be coupled to a cell-penetrating peptide (CPP), and labeling cells may comprise delivering the CPP coupled reporter oligonucleotide into an analyte carrier. Labeling analyte carriers may comprise delivering the CPP conjugated oligonucleotide into a cell and/or cell bead by the cell-penetrating peptide. A cell-penetrating peptide that can be used in the methods provided herein can comprise at least one non-functional cysteine residue, which may be either free or derivatized to form a disulfide link with an oligonucleotide that has been modified for such linkage. Non-limiting examples of cell-penetrating peptides that can be used in instances herein include penetratin, transportan, plsl, TAT (48-60), pVEC, MTS, and MAP. Cell-penetrating peptides useful in the methods provided herein can have the capability of inducing cell penetration for at least about 30%, 40%, 50%, 60%, 70%, 80%, 90%, 95%, 96%, 97%, 98%, 99%, or 100% of cells of a cell population. The cell-penetrating peptide may be an arginine-rich peptide transporter. The cell-penetrating peptide may be Penetratin or the Tat peptide.

In another example, a reporter oligonucleotide may be coupled to a fluorophore or dye, and labeling cells may comprise subjecting the fluorophore-linked barcode molecule to conditions suitable for binding the fluorophore to the surface of the analyte carrier. In some instances, fluorophores can interact strongly with lipid bilayers and labeling analyte carriers may comprise subjecting the fluorophore-linked barcode molecule to conditions such that the fluorophore binds to or is inserted into a membrane of the analyte carrier. In some cases, the fluorophore is a water-soluble, organic fluorophore. In some instances, the fluorophore is Alexa 532 maleimide, tetramethylrhodamine-5-maleimide (TMR maleimide), BODIPY-TMR maleimide, Sulfo-Cy3 maleimide, Alexa 546 carboxylic acid/succinimidyl ester, Atto 550 maleimide, Cy3 carboxylic acid/succinimidyl ester, Cy3B carboxylic acid/succinimidyl ester, Atto 565 biotin, Sulforhodamine B, Alexa 594 maleimide, Texas Red maleimide, Alexa 633 maleimide, Abberior STAR 635P azide, Atto 647N maleimide, Atto 647 SE, or Sulfo-Cy5 maleimide. Sec, e.g., Hughes L D, et al. PloS One. 2014 Feb. 4; 9(2) e87649, which is hereby incorporated by reference in its entirety for all purposes, for a description of organic fluorophores.

A reporter oligonucleotide may be coupled to a lipophilic molecule, and labeling analyte carriers may comprise delivering the nucleic acid barcode molecule to a membrane of the analyte carrier or a nuclear membrane by the lipophilic molecule. Lipophilic molecules can associate with and/or insert into lipid membranes such as cell membranes and nuclear membranes. In some cases, the insertion can be reversible. In some cases, the association between the lipophilic molecule and analyte carrier may be such that the analyte carrier retains the lipophilic molecule (e.g., and associated components, such as nucleic acid barcode molecules, thereof) during subsequent processing (e.g., partitioning, cell permeabilization, amplification, pooling, etc.) The reporter nucleotide may enter into the intracellular space and/or a cell nucleus.

A reporter oligonucleotide may be part of a nucleic acid molecule comprising any number of functional sequences, as described elsewhere herein, such as a target capture sequence, a random primer sequence, and the like, and coupled to another nucleic acid molecule that is, or is derived from, the analyte.

Prior to partitioning, the cells may be incubated with the library of labelling agents, that may be labelling agents to a broad panel of different cell features, e.g., receptors, proteins, etc., and which include their associated reporter oligonucleotides. Unbound labelling agents may be washed from the cells, and the cells may then be co-partitioned (e.g., into droplets or wells) along with partition-specific barcode oligonucleotides (e.g., attached to a support, such as a bead or gel bead) as described elsewhere herein. As a result, the partitions may include the cell or cells, as well as the bound labelling agents and their known, associated reporter oligonucleotides.

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide. For example, the first plurality of the labeling agent and second plurality of the labeling agent may interact with different cells, cell populations or samples, allowing a particular report oligonucleotide to indicate a particular cell population (or cell or sample) and cell feature. In this way, different samples or groups can be independently processed and subsequently combined together for pooled analysis (e.g., partition-based barcoding as described elsewhere herein). See, e.g., U.S. Pat. Pub. 20190323088, which is hereby entirely incorporated by reference for all purposes.

As described elsewhere herein, libraries of labelling agents may be associated with a particular cell feature as well as be used to identify analytes as originating from a particular analyte carrier, population, or sample. The analyte carriers may be incubated with a plurality of libraries and a given analyte carrier may comprise multiple labelling agents. For example, a cell may comprise coupled thereto a lipophilic labeling agent and an antibody. The lipophilic labeling agent may indicate that the cell is a member of a particular cell sample, whereas the antibody may indicate that the cell comprises a particular analyte. In this manner, the reporter oligonucleotides and labelling agents may allow multi-analyte, multiplexed analyses to be performed.

In some instances, these reporter oligonucleotides may comprise nucleic acid barcode sequences that permit identification of the labelling agent which the reporter oligonucleotide is coupled to. The use of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents may be achieved through any of a variety of direct or indirect, covalent or non-covalent associations or attachments. For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2): 708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry such as a Methyltetrazine-PEG5-NHS Ester reaction, a TCO-PEG4-NHS Ester reaction, or the like, may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some instances, the reporter oligonucleotides are releasable from the labelling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a first primer or primer binding sequence, a sequencing primer or primer binding sequence (such as an R1, R2, or partial R1 or R2 sequence).

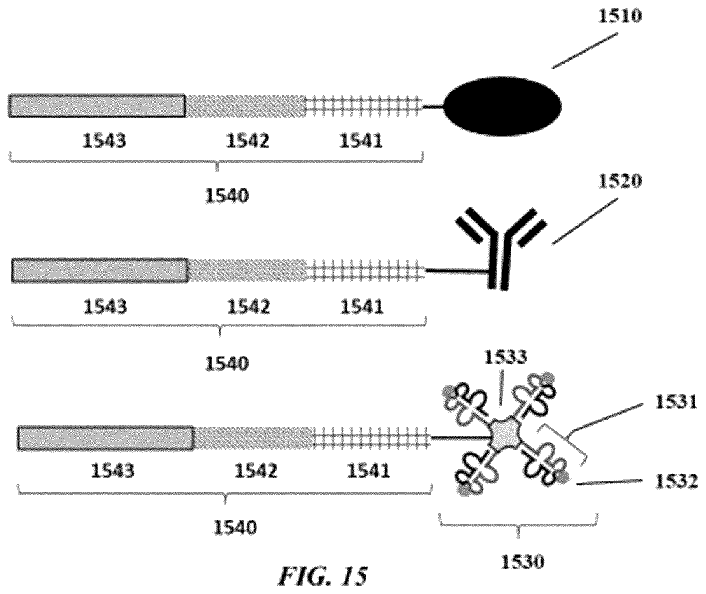

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to an oligonucleotide that is complementary to a sequence of the reporter oligonucleotide, and the oligonucleotide may be allowed to hybridize to the reporter oligonucleotide. FIG. 15 describes example labelling agents (1510, 1520, 1530) comprising reporter oligonucleotides (1540) attached thereto. Labelling agent 1510 (e.g., any of the labelling agents described herein) is attached (either directly, e.g., covalently attached, or indirectly) to reporter oligonucleotide 1540. Reporter oligonucleotide 1540 may comprise barcode sequence 1542 that identifies labelling agent 1510. Reporter oligonucleotide 1540 may also comprise one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a first primer or primer binding sequence, or a sequencing primer or primer binding sequence (such as an R1, R2, or partial R1 or R2 sequence).

Referring to FIG. 15, in some instances, reporter oligonucleotide 1540 conjugated to a labelling agent (e.g., 1510, 1520, 1530) comprises a primer sequence 1541, a barcode sequence that identifies the labelling agent (e.g., 1510, 1520, 1530), and functional sequence 1543. Functional sequence 1543 may be configured to hybridize to a complementary sequence, such as a complementary sequence present on a nucleic acid barcode molecule 1590 (not shown), such as those described elsewhere herein. In some instances, nucleic acid barcode molecule 1590 is attached to a support (e.g., a bead, such as a gel bead), such as those described elsewhere herein. For example, nucleic acid barcode molecule 1590 may be attached to the support via a releasable linkage (e.g., comprising a labile bond), such as those described elsewhere herein. In some instances, reporter oligonucleotide 1540 comprises one or more additional functional sequences, such as those described above.

In some instances, the labelling agent 1510 is a protein or polypeptide (e.g., an antigen or prospective antigen) comprising reporter oligonucleotide 1540. Reporter oligonucleotide 1540 comprises barcode sequence 1542 that identifies polypeptide 1510 and can be used to infer the presence of an analyte, e.g., a binding partner of polypeptide 1510 (e.g., a molecule or compound to which polypeptide 1510 can bind). In some instances, the labelling agent 1510 is a lipophilic moiety (e.g., cholesterol) comprising reporter oligonucleotide 1540, where the lipophilic moiety is selected such that labelling agent 1510 integrates into a membrane of a cell or nucleus. Reporter oligonucleotide 1540 comprises barcode sequence 1542 that identifies lipophilic moiety 1510 which in some instances is used to tag cells (e.g., groups of cells, cell samples, etc.) and may be used for multiplex analyses as described elsewhere herein. In some instances, the labelling agent is an antibody 1520 (or an epitope binding fragment thereof) comprising reporter oligonucleotide 1540. Reporter oligonucleotide 1540 comprises barcode sequence 1542 that identifies antibody 1520 and can be used to infer the presence of, e.g., a target of antibody 1520 (e.g., a molecule or compound to which antibody 1520 binds). In other cases, labelling agent 1530 comprises an MHC molecule 1531 comprising peptide 1532 and reporter oligonucleotide 1540 that identifies peptide 1532. In some instances, the MHC molecule is coupled to a support 1533. In some instances, support 1533 may be a polypeptide, such as streptavidin, or a polysaccharide, such as dextran. In some instances, reporter oligonucleotide 1540 may be directly or indirectly coupled to MHC labelling agent 1530 in any suitable manner. For example, reporter oligonucleotide 1540 may be coupled to MHC molecule 1531, support 1533, or peptide 1532. In some instances, labelling agent 1530 comprises a plurality of MHC molecules, (e.g. Is an MHC multimer, which may be coupled to a support (e.g., 1533)). There are many possible configurations of Class I and/or Class II MHC multimers that can be utilized with the compositions, methods, and systems disclosed herein, e.g., MHC tetramers, MHC pentamers (MHC assembled via a coiled-coil domain, e.g., Pro5® MHC Class I Pentamers, (ProImmune, Ltd.), MHC octamers, MHC dodecamers, MHC decorated dextran molecules (e.g., MHC Dextramer® (Immudex)), etc. For a description of example labelling agents, including antibody and MHC-based labelling agents, reporter oligonucleotides, and methods of use, see, e.g., U.S. Pat. No. 10,550,429 and U.S. Pat. Pub. 20190367969, each of which is herein entirely incorporated by reference for all purposes.

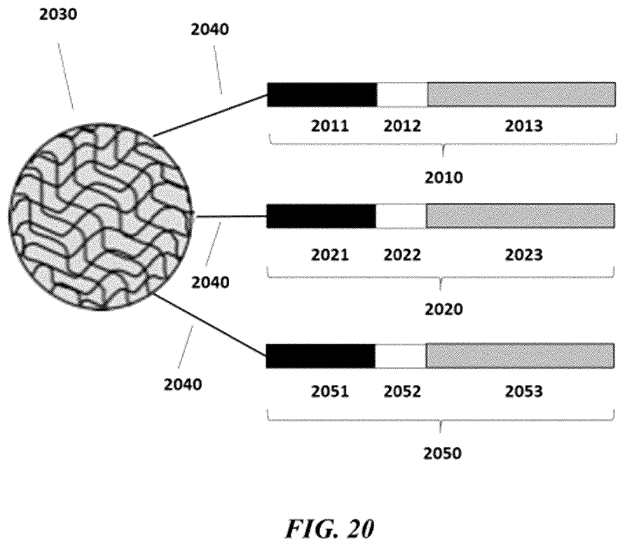

FIG. 20 illustrates another example of a barcode carrying bead. In some embodiments, analysis of multiple analytes (e.g., RNA and one or more analytes using labelling agents described herein) may comprise nucleic acid barcode molecules as generally depicted in FIG. 20. In some embodiments, nucleic acid barcode molecules 2010 and 2020 are attached to support 2030 via a releasable linkage 2040 (e.g., comprising a labile bond) as described elsewhere herein. Nucleic acid barcode molecule 2010 may comprise adapter sequence 2011, barcode sequence 2012 and adapter sequence 2013. Nucleic acid barcode molecule 2020 may comprise adapter sequence 2021, barcode sequence 2022, and adapter sequence 2023, wherein adapter sequence 2023 comprises a different sequence than adapter sequence 2013. In some instances, adapter 2011 and adapter 2021 comprise the same sequence. In some instances, adapter 2011 and adapter 2021 comprise different sequences. Although support 2030 is shown comprising nucleic acid barcode molecules 2010 and 2020, any suitable number of barcode molecules comprising common barcode sequence 2012 are contemplated herein. For example, in some embodiments, support 2030 further comprises nucleic acid barcode molecule 2050. Nucleic acid barcode molecule 2050 may comprise adapter sequence 2051, barcode sequence 2012 and adapter sequence 2053, wherein adapter sequence 2053 comprises a different sequence than adapter sequence 2013 and 2023. In some instances, nucleic acid barcode molecules (e.g., 2010, 2020, 2050) comprise one or more additional functional sequences, such as a UMI or other sequences described herein. The nucleic acid barcode molecules 2010, 2020, or 2050 may interact with analytes as described elsewhere herein, for example, as depicted in FIGS. 16A-C.

Referring to FIG. 16A, in an instance where cells are labelled with labeling agents, sequence 1623 may be complementary to an adapter sequence of a reporter oligonucleotide. Cells may be contacted with one or more reporter oligonucleotide 1620 conjugated labelling agents 1610 (e.g., polypeptide, antibody, or others described elsewhere herein). In some cases, the cells may be further processed prior to barcoding. For example, such processing operations may include one or more washing and/or cell sorting operations. In some instances, a cell that is bound to labelling agent 1610 which is conjugated to oligonucleotide 1620 and support 1630 (e.g., a bead, such as a gel bead) comprising nucleic acid barcode molecule 1690 is partitioned into a partition amongst a plurality of partitions (e.g., a droplet of a droplet emulsion or a well of a microwell array). In some instances, the partition comprises at most a single cell bound to labelling agent 1610. In some instances, reporter oligonucleotide 1620 conjugated to labelling agent 1610 (e.g., polypeptide, an antibody, pMHC molecule such as an MHC multimer, etc.) comprises a first adapter sequence 1611 (e.g., a primer sequence), a barcode sequence 1612 that identifies the labelling agent 1610 (e.g., the polypeptide, antibody, or peptide of a pMHC molecule or complex), and an adapter sequence 1613. Adapter sequence 1613 may be configured to hybridize to a complementary sequence, such as sequence 1623 present on a nucleic acid barcode molecule 1690. In some instances, oligonucleotide 1620 comprises one or more additional functional sequences, such as those described elsewhere herein.

Barcoded nucleic acids may be generated (e.g., via a nucleic acid reaction, such as nucleic acid extension or ligation) from the constructs described in FIGS. 16A-C. For example, sequence 1613 may then be hybridized to complementary sequence 1623 to generate (e.g., via a nucleic acid reaction, such as nucleic acid extension or ligation) a barcoded nucleic acid molecule comprising cell (e.g., partition specific) barcode sequence 1622 (or a reverse complement thereof) and reporter barcode sequence 1612 (or a reverse complement thereof). Barcoded nucleic acid molecules can then be optionally processed as described elsewhere herein, e.g., to amplify the molecules and/or append sequencing platform specific sequences to the fragments. See, e.g., U.S. Pat. Pub. 2018/0105808, which is hereby entirely incorporated by reference for all purposes. Barcoded nucleic acid molecules, or derivatives generated therefrom, can then be sequenced on a suitable sequencing platform.

In some instances, analysis of multiple analytes (e.g., nucleic acids and one or more analytes using labelling agents described herein) may be performed. For example, the workflow may comprise a workflow as generally depicted in any of FIGS. 16A-C, or a combination of workflows for an individual analyte, as described elsewhere herein. For example, by using a combination of the workflows as generally depicted in FIG. 16A-C, multiple analytes can be analyzed.

In some instances, analysis of an analyte (e.g. A nucleic acid, a polypeptides, a carbohydrate, a lipid, etc.) comprises a workflow as generally depicted in FIG. 16A. A nucleic acid barcode molecule 1690 may be co-partitioned with the one or more analytes. In some instances, nucleic acid barcode molecule 1690 is attached to a support 1630 (e.g., a bead, such as a gel bead), such as those described elsewhere herein. For example, nucleic acid barcode molecule 1690 may be attached to support 1630 via a releasable linkage 1640 (e.g., comprising a labile bond), such as those described elsewhere herein. Nucleic acid barcode molecule 1690 may comprise a barcode sequence 1621 and optionally comprise other additional sequences, for example, a UMI sequence 1622 (or other functional sequences described elsewhere herein). The nucleic acid barcode molecule 1690 may comprise a sequence 1623 that may be complementary to another nucleic acid sequence, such that it may hybridize to a particular sequence.

For example, sequence 1623 may comprise a poly-T sequence and may be used to hybridize to mRNA. Referring to FIG. 16C, in some cases, nucleic acid barcode molecule 1690 comprises sequence 1623 complementary to a sequence of RNA molecule 1660 from a cell. In some instances, sequence 1623 comprises a sequence specific for an RNA molecule. Sequence 1623 may comprise a known or targeted sequence or a random sequence. In some instances, a nucleic acid extension reaction may be performed, thereby generating a barcoded nucleic acid product comprising sequence 1623, the barcode sequence 1621, UMI sequence 1622, any other functional sequence, and a sequence corresponding to the RNA molecule 1660.

In another example, sequence 1623 may be complementary to an overhang sequence or an adapter sequence that has been appended to an analyte. For example, referring to FIG. 16B, panel 1601, in some instances, primer 1650 comprises a sequence complementary to a sequence of nucleic acid molecule 1660 (such as an RNA encoding for a BCR sequence) from an analyte carrier. In some instances, primer 1650 comprises one or more sequences 1651 that are not complementary to RNA molecule 1660. Sequence 1651 may be a functional sequence as described elsewhere herein, for example, an adapter sequence, a sequencing primer sequence, or a sequence the facilitates coupling to a flow cell of a sequencer. In some instances, primer 1650 comprises a poly-T sequence. In some instances, primer 1650 comprises a sequence complementary to a target sequence in an RNA molecule. In some instances, primer 1650 comprises a sequence complementary to a region of an immune molecule, such as the constant region of a TCR or BCR sequence. Primer 1650 is hybridized to nucleic acid molecule 1660 and complementary molecule 1670 is generated (see Panel 1602). For example, complementary molecule 1670 may be cDNA generated in a reverse transcription reaction. In some instances, an additional sequence may be appended to complementary molecule 1670. For example, the reverse transcriptase enzyme may be selected such that several non-templated bases 1680 (e.g., a poly-C sequence) are appended to the cDNA. In another example, a terminal transferase may also be used to append the additional sequence. Nucleic acid barcode molecule 1690 comprises a sequence 1624 complementary to the non-templated bases, and the reverse transcriptase performs a template switching reaction onto nucleic acid barcode molecule 1690 to generate a barcoded nucleic acid molecule comprising cell (e.g., partition specific) barcode sequence 1622 (or a reverse complement thereof) and a sequence of complementary molecule 1670 (or a portion thereof). In some instances, sequence 1623 comprises a sequence complementary to a region of an immune molecule, such as the constant region of a TCR or BCR sequence. Sequence 1623 is hybridized to nucleic acid molecule 1660 and a complementary molecule 1670 is generated. For example complementary molecule 1670 may be generated in a reverse transcription reaction generating a barcoded nucleic acid molecule comprising cell (e.g., partition specific) barcode sequence 1622 (or a reverse complement thereof) and a sequence of complementary molecule 1670 (or a portion thereof). Additional methods and compositions suitable for barcoding cDNA generated from mRNA transcripts including those encoding V(D)J regions of an immune cell receptor and/or barcoding methods and composition including a template switch oligonucleotide are described in International Patent Application WO2018/075693, U.S. Patent Publication No 2018/0105808, U.S. Patent Publication No. 2015/0376609, filed Jun. 26, 2015, and U.S. Patent Publication No. 2019/0367969, each of which applications is herein entirely incorporated by reference for all purposes.

### Reagents

In accordance with certain aspects, biological particles may be partitioned along with lysis reagents in order to release the contents of the biological particles within the partition. In such cases, the lysis agents can be contacted with the biological particle suspension concurrently with, or immediately prior to, the introduction of the biological particles into the partitioning junction/droplet generation zone (e.g., junction 210), such as through an additional channel or channels upstream of the channel junction. In accordance with other aspects, additionally or alternatively, biological particles may be partitioned along with other reagents, as will be described further below.

The methods and systems of the present disclosure may comprise microfluidic devices and methods of use thereof, which may be used for co-partitioning analyte carriers or analyte carriers with reagents. Such systems and methods are described in U.S. Patent Publication No. US/20190367997, which is herein incorporated by reference in its entirety for all purposes.

Beneficially, when lysis reagents and biological particles are co-partitioned, the lysis reagents can facilitate the release of the contents of the biological particles within the partition. The contents released in a partition may remain discrete from the contents of other partitions.

As will be appreciated, the channel segments of the microfluidic devices described elsewhere herein may be coupled to any of a variety of different fluid sources or receiving components, including reservoirs, tubing, manifolds, or fluidic components of other systems. As will be appreciated, the microfluidic channel structures may have various geometries and/or configurations. For example, a microfluidic channel structure can have more than two channel junctions. For example, a microfluidic channel structure can have 2, 3, 4, 5 channel segments or more each carrying the same or different types of beads, reagents, and/or biological particles that meet at a channel junction. Fluid flow in each channel segment may be controlled to control the partitioning of the different elements into droplets. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, MO), as well as other commercially available lysis enzymes. Other lysis agents may additionally or alternatively be co-partitioned with the biological particles to cause the release of the biological particle's contents into the partitions. For example, in some cases, surfactant-based lysis solutions may be used to lyse cells. In some cases, lysis solutions may include non-ionic surfactants such as, for example, TritonX-100 and Tween 20. In some cases, lysis solutions may include ionic surfactants such as, for example, sarcosyl and sodium dodecyl sulfate (SDS). Electroporation, thermal, acoustic or mechanical cellular disruption may also be used in certain cases, e.g., non-emulsion based partitioning such as encapsulation of biological particles that may be in addition to or in place of droplet partitioning, where any pore size of the encapsulate is sufficiently small to retain nucleic acid fragments of a given size, following cellular disruption.

Alternatively or in addition to the lysis agents co-partitioned with the analyte carriers described above, other reagents can also be co-partitioned with the analyte carriers, including, for example, Dnase and Rnase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated analyte carriers (e.g., a cell or a nucleus in a polymer matrix), the analyte carriers may be exposed to an appropriate stimulus to release the analyte carriers or their contents from a co-partitioned microcapsule. For example, in some cases, a chemical stimulus may be co-partitioned along with an encapsulated analyte carrier to allow for the degradation of the microcapsule and release of the cell or its contents into the larger partition. In some cases, this stimulus may be the same as the stimulus described elsewhere herein for release of nucleic acid molecules (e.g., oligonucleotides) from their respective microcapsule (e.g., bead). In alternative examples, this may be a different and non-overlapping stimulus, in order to allow an encapsulated analyte carrier to be released into a partition at a different time from the release of nucleic acid molecules into the same partition. For a description of methods, compositions, and systems for encapsulating cells (also referred to as a “cell bead”), see, e.g., U.S. Pat. No. 10,428,326 and U.S. Pat. Pub. 20190100632, which are each incorporated by reference in their entirety.

Additional reagents may also be co-partitioned with the biological particles, such as endonucleases to fragment a biological particle's DNA, DNA polymerase enzymes and dNTPs used to amplify the biological particle's nucleic acid fragments and to attach the barcode molecular tags to the amplified fragments. Other enzymes may be co-partitioned, including without limitation, polymerase, transposase, ligase, proteinase K, DNAse, etc. Additional reagents may also include reverse transcriptase enzymes, including enzymes with terminal transferase activity, primers and oligonucleotides, and switch oligonucleotides (also referred to herein as “switch oligos” or “template switching oligonucleotides”) which can be used for template switching. In some cases, template switching can be used to increase the length of a cDNA. In some cases, template switching can be used to append a predefined nucleic acid sequence to the cDNA. In an example of template switching, cDNA can be generated from reverse transcription of a template, e.g., cellular mRNA, where a reverse transcriptase with terminal transferase activity can add additional nucleotides, e.g., polyC, to the cDNA in a template independent manner. Switch oligos can include sequences complementary to the additional nucleotides, e.g., polyG. The additional nucleotides (e.g., polyC) on the cDNA can hybridize to the additional nucleotides (e.g., polyG) on the switch oligo, whereby the switch oligo can be used by the reverse transcriptase as template to further extend the cDNA. Template switching oligonucleotides may comprise a hybridization region and a template region. The hybridization region can comprise any sequence capable of hybridizing to the target. In some cases, the hybridization region comprises a series of G bases to complement the overhanging C bases at the 3′ end of a cDNA molecule. The series of G bases may comprise 1 G base, 2 G bases, 3 G bases, 4 G bases, 5 G bases or more than 5 G bases. The template sequence can comprise any sequence to be incorporated into the cDNA. In some cases, the template region comprises at least 1 (e.g., at least 2, 3, 4, 5 or more) tag sequences and/or functional sequences. Switch oligos may comprise deoxyribonucleic acids; ribonucleic acids; modified nucleic acids including 2-Aminopurine, 2,6-Diaminopurine (2-Amino-dA), inverted dT, 5-Methyl dC, 2′-deoxyInosine, Super T (5-hydroxybutynl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′ Fluoro bases (e.g., Fluoro C, Fluoro U, Fluoro A, and Fluoro G), or any combination.

In some cases, the length of a switch oligo may be at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides or longer.

In some cases, the length of a switch oligo may be at most about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides.

Once the contents of the cells are released into their respective partitions, the macromolecular components (e.g., macromolecular constituents of biological particles, such as RNA, DNA, or proteins) contained therein may be further processed within the partitions. In accordance with the methods and systems described herein, the macromolecular component contents of individual biological particles can be provided with unique identifiers such that, upon characterization of those macromolecular components they may be attributed as having been derived from the same biological particle or particles. The ability to attribute characteristics to individual biological particles or groups of biological particles is provided by the assignment of unique identifiers specifically to an individual biological particle or groups of biological particles. Unique identifiers, e.g., in the form of nucleic acid barcodes can be assigned or associated with individual biological particles or populations of biological particles, in order to tag or label the biological particle's macromolecular components (and as a result, its characteristics) with the unique identifiers. These unique identifiers can then be used to attribute the biological particle's components and characteristics to an individual biological particle or group of biological particles.

In some aspects, this is performed by co-partitioning the individual biological particle or groups of biological particles with the unique identifiers, such as described above (with reference to FIG. 2). In some aspects, the unique identifiers are provided in the form of nucleic acid molecules (e.g., oligonucleotides) that comprise nucleic acid barcode sequences that may be attached to or otherwise associated with the nucleic acid contents of individual biological particle, or to other components of the biological particle, and particularly to fragments of those nucleic acids. The nucleic acid molecules are partitioned such that as between nucleic acid molecules in a given partition, the nucleic acid barcode sequences contained therein are the same, but as between different partitions, the nucleic acid molecule can, and do have differing barcode sequences, or at least represent a large number of different barcode sequences across all of the partitions in a given analysis. In some aspects, only one nucleic acid barcode sequence can be associated with a given partition, although in some cases, two or more different barcode sequences may be present.

The nucleic acid barcode sequences can include from about 6 to about 20 or more nucleotides within the sequence of the nucleic acid molecules (e.g., oligonucleotides). The nucleic acid barcode sequences can include from about 6 to about 20, 30, 40, 50, 60, 70, 80, 90, 100 or more nucleotides. In some cases, the length of a barcode sequence may be about 6, 7, 8, 9, 10, 11, 12, 13, 14, 15, 16, 17, 18, 19, 20 nucleotides or longer. In some cases, the length of a barcode sequence may be at least about 6, 7, 8, 9, 10, 11, 12, 13, 14, 15, 16, 17, 18, 19, 20 nucleotides or longer. In some cases, the length of a barcode sequence may be at most about 6, 7, 8, 9, 10, 11, 12, 13, 14, 15, 16, 17, 18, 19, 20 nucleotides or shorter. These nucleotides may be completely contiguous, e.g., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be about 4, 5, 6, 7, 8, 9, 10, 11, 12, 13, 14, 15, 16 nucleotides or longer. In some cases, the barcode subsequence may be at least about 4, 5, 6, 7, 8, 9, 10, 11, 12, 13, 14, 15, 16 nucleotides or longer. In some cases, the barcode subsequence may be at most about 4, 5, 6, 7, 8, 9, 10, 11, 12, 13, 14, 15, 16 nucleotides or shorter.

The co-partitioned nucleic acid molecules can also comprise other functional sequences useful in the processing of the nucleic acids from the co-partitioned biological particles. These sequences include, e.g., targeted or random/universal amplification primer sequences for amplifying nucleic acids (e.g., mRNA, the genomic DNA) from the individual biological particles within the partitions while attaching the associated barcode sequences, sequencing primers or primer recognition sites, hybridization or probing sequences, e.g., for identification of presence of the sequences or for pulling down barcoded nucleic acids, or any of a number of other potential functional sequences. Other mechanisms of co-partitioning oligonucleotides may also be employed, including, e.g., coalescence of two or more droplets, where one droplet contains oligonucleotides, or microdispensing of oligonucleotides (e.g., attached to a bead) into partitions, e.g., droplets within microfluidic systems.

In an example, microcapsules, such as beads, are provided that each include large numbers of the above described barcoded nucleic acid molecules (e.g., barcoded oligonucleotides) releasably attached to the beads, where all of the nucleic acid molecules attached to a particular bead will include the same nucleic acid barcode sequence, but where a large number of diverse barcode sequences are represented across the population of beads used. In some cases, hydrogel beads, e.g., comprising polyacrylamide polymer matrices, are used as a solid support and delivery vehicle for the nucleic acid molecules into the partitions, as they are capable of carrying large numbers of nucleic acid molecules, and may be configured to release those nucleic acid molecules upon exposure to a particular stimulus, as described elsewhere herein. In some cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. Additionally, each bead can be provided with large numbers of nucleic acid (e.g., oligonucleotide) molecules attached. In particular, the number of molecules of nucleic acid molecules including the barcode sequence on an individual bead can be at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules, or more. Nucleic acid molecules of a given bead can include identical (or common) barcode sequences, different barcode sequences, or a combination of both. Nucleic acid molecules of a given bead can include multiple sets of nucleic acid molecules. Nucleic acid molecules of a given set can include identical barcode sequences. The identical barcode sequences can be different from barcode sequences of nucleic acid molecules of another set.

Moreover, when the population of beads is partitioned, the resulting population of partitions can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules.

In some cases, multiple different barcodes can be incorporated within a given partition, either attached to a single or multiple beads within the partition. For example, in some cases, a mixed, but known set of barcode sequences may provide greater assurance of identification in the subsequent processing, e.g., by providing a stronger address or attribution of the barcodes to a given partition, as a duplicate or independent confirmation of the output from a given partition.

The nucleic acid molecules (e.g., oligonucleotides) are releasable from the beads upon the application of a particular stimulus to the beads. In some cases, the stimulus may be a photo-stimulus, e.g., through cleavage of a photo-labile linkage that releases the nucleic acid molecules. In other cases, a thermal stimulus may be used, where elevation of the temperature of the beads environment will result in cleavage of a linkage or other release of the nucleic acid molecules from the beads. In still other cases, a chemical stimulus can be used that cleaves a linkage of the nucleic acid molecules to the beads, or otherwise results in release of the nucleic acid molecules from the beads. In one case, such compositions include the polyacrylamide matrices described above for encapsulation of biological particles, and may be degraded for release of the attached nucleic acid molecules through exposure to a reducing agent, such as DTT.

In some aspects, provided are systems and methods for controlled partitioning. Droplet size may be controlled by adjusting certain geometric features in channel architecture (e.g., microfluidics channel architecture). For example, an expansion angle, width, and/or length of a channel may be adjusted to control droplet size.

FIG. 2 shows an example of a microfluidic channel structure for the controlled partitioning of beads into discrete droplets. A channel structure 200 can include a channel segment 202 communicating at a channel junction 206 (or intersection) with a reservoir 204. The reservoir 204 can be a chamber. Any reference to “reservoir,” as used herein, can also refer to a “chamber.” In operation, an aqueous fluid 208 that includes suspended beads 212 may be transported along the channel segment 202 into the junction 206 to meet a second fluid 210 that is immiscible with the aqueous fluid 208 in the reservoir 204 to create droplets 216, 218 of the aqueous fluid 208 flowing into the reservoir 204. At the junction 206 where the aqueous fluid 208 and the second fluid 210 meet, droplets can form based on factors such as the hydrodynamic forces at the junction 206, flow rates of the two fluids 208, 210, fluid properties, and certain geometric parameters (e.g., w, ho, a, etc.) of the channel structure 200. A plurality of droplets can be collected in the reservoir 204 by continuously injecting the aqueous fluid 208 from the channel segment 202 through the junction 206.

A discrete droplet generated may include a bead (e.g., as in occupied droplets 216). Alternatively, a discrete droplet generated may include more than one bead. Alternatively, a discrete droplet generated may not include any beads (e.g., as in unoccupied droplet 218). In some instances, a discrete droplet generated may contain one or more analyte carriers, as described elsewhere herein. In some instances, a discrete droplet generated may comprise one or more reagents, as described elsewhere herein.

In some instances, the aqueous fluid 208 can have a substantially uniform concentration or frequency of beads 212. The beads 212 can be introduced into the channel segment 202 from a separate channel (not shown in FIG. 2). The frequency of beads 212 in the channel segment 202 may be controlled by controlling the frequency in which the beads 212 are introduced into the channel segment 202 and/or the relative flow rates of the fluids in the channel segment 202 and the separate channel. In some instances, the beads can be introduced into the channel segment 202 from a plurality of different channels, and the frequency controlled accordingly.

In some instances, the aqueous fluid 208 in the channel segment 202 can comprise biological particles. In some instances, the aqueous fluid 208 can have a substantially uniform concentration or frequency of biological particles. As with the beads, the biological particles can be introduced into the channel segment 202 from a separate channel. The frequency or concentration of the biological particles in the aqueous fluid 208 in the channel segment 202 may be controlled by controlling the frequency in which the biological particles are introduced into the channel segment 202 and/or the relative flow rates of the fluids in the channel segment 202 and the separate channel. In some instances, the biological particles can be introduced into the channel segment 202 from a plurality of different channels, and the frequency controlled accordingly. In some instances, a first separate channel can introduce beads and a second separate channel can introduce biological particles into the channel segment 202. The first separate channel introducing the beads may be upstream or downstream of the second separate channel introducing the biological particles.

The second fluid 210 can comprise an oil, such as a fluorinated oil, that includes a fluorosurfactant for stabilizing the resulting droplets, for example, inhibiting subsequent coalescence of the resulting droplets.

In some instances, the second fluid 210 may not be subjected to and/or directed to any flow in or out of the reservoir 204. For example, the second fluid 210 may be substantially stationary in the reservoir 204. In some instances, the second fluid 210 may be subjected to flow within the reservoir 204, but not in or out of the reservoir 204, such as via application of pressure to the reservoir 204 and/or as affected by the incoming flow of the aqueous fluid 208 at the junction 206. Alternatively, the second fluid 210 may be subjected and/or directed to flow in or out of the reservoir 204. For example, the reservoir 204 can be a channel directing the second fluid 210 from upstream to downstream, transporting the generated droplets.

The channel structure 200 at or near the junction 206 may have certain geometric features that at least partly determine the sizes of the droplets formed by the channel structure 200. The channel segment 202 can have a height, ho and width, w, at or near the junction 206. By way of example, the channel segment 202 can comprise a rectangular cross-section that leads to a reservoir 204 having a wider cross-section (such as in width or diameter). Alternatively, the cross-section of the channel segment 202 can be other shapes, such as a circular shape, trapezoidal shape, polygonal shape, or any other shapes. The top and bottom walls of the reservoir 204 at or near the junction 206 can be inclined at an expansion angle, a. The expansion angle, a, allows the tongue (portion of the aqueous fluid 208 leaving channel segment 202 at junction 206 and entering the reservoir 204 before droplet formation) to increase in depth and facilitate decrease in curvature of the intermediately formed droplet. Droplet size may decrease with increasing expansion angle. The resulting droplet radius, Ra, may be predicted by the following equation for the aforementioned geometric parameters of ho, w, and a:

$${R}_{d} \approx 0.44 ( 1 + 2.2 \sqrt{tan α} \frac{w}{{h}_{0}} ) \frac{{h}_{0}}{\sqrt{tan α}}$$

By way of example, for a channel structure with w=21 μm, h=21 μm, and α=3°, the predicted droplet size is 121 μm. In another example, for a channel structure with w=25 μm, h=25 μm, and α=5°, the predicted droplet size is 123 μm. In another example, for a channel structure with w=28 μm, h=28 μm, and α=7°, the predicted droplet size is 124 μm.

In some instances, the expansion angle, a, may be between a range of from about 0.5° to about 4°, from about 0.1° to about 10°, or from about 0° to about 90°. For example, the expansion angle can be at least about 0.01°, 0.1°, 0.2°, 0.3°, 0.4°, 0.5°, 0.6°, 0.7°, 0.8°, 0.9°, 1°, 2°, 3°, 4°, 5°, 6°, 7°, 8°, 9°, 10°, 15°, 20°, 25°, 30°, 35°, 40°, 45°, 50°, 55°, 60°, 65°, 70°, 75°, 80°, 85°, or higher. In some instances, the expansion angle can be at most about 89°, 88°, 87°, 86°, 85°, 84°, 83°, 82°, 81°, 80°, 75°, 70°, 65°, 60°, 55°, 50°, 45°, 40°, 35°, 30°, 25°, 20°, 15°, 10°, 9°, 8°, 7°, 6°, 5°, 4°, 3°, 2°, 1°, 0.1°, 0.01°, or less. In some instances, the width, w, can be between a range of from about 100 micrometers (μm) to about 500 μm. In some instances, the width, w, can be between a range of from about 10 μm to about 200 μm. Alternatively, the width can be less than about 10 μm. Alternatively, the width can be greater than about 500 μm. In some instances, the flow rate of the aqueous fluid 208 entering the junction 206 can be between about 0.04 microliters (μL)/minute (min) and about 40 μL/min. In some instances, the flow rate of the aqueous fluid 208 entering the junction 206 can be between about 0.01 microliters (μL)/minute (min) and about 100 μL/min. Alternatively, the flow rate of the aqueous fluid 208 entering the junction 206 can be less than about 0.01 μL/min. Alternatively, the flow rate of the aqueous fluid 208 entering the junction 206 can be greater than about 40 μL/min, such as 45 μL/min, 50 μL/min, 55 μL/min, 60 μL/min, 65 μL/min, 70 μL/min, 75 μL/min, 80 μL/min, 85 L/min, 90 μL/min, 95 μL/min, 100 μL/min, 110 μL/min, 120 μL/min, 130 μL/min, 140 μL/min, 150 μL/min, or greater. At lower flow rates, such as flow rates of about less than or equal to 10 microliters/minute, the droplet radius may not be dependent on the flow rate of the aqueous fluid 208 entering the junction 206.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

The throughput of droplet generation can be increased by increasing the points of generation, such as increasing the number of junctions (e.g., junction 206) between aqueous fluid 208 channel segments (e.g., channel segment 202) and the reservoir 204. Alternatively or in addition, the throughput of droplet generation can be increased by increasing the flow rate of the aqueous fluid 208 in the channel segment 202.

The methods and systems described herein may be used to greatly increase the efficiency of single cell applications and/or other applications receiving droplet-based input. For example, following the sorting of occupied cells and/or appropriately-sized cells, subsequent operations that can be performed can include generation of amplification products, purification (e.g., via solid phase reversible immobilization (SPRI)), further processing (e.g., shearing, ligation of functional sequences, and subsequent amplification (e.g., via PCR)). These operations may occur in bulk (e.g., outside the partition). In the case where a partition is a droplet in an emulsion, the emulsion can be broken and the contents of the droplet pooled for additional operations. Additional reagents that may be co-partitioned along with the barcode bearing bead may include oligonucleotides to block ribosomal RNA (rRNA) and nucleases to digest genomic DNA from cells. Alternatively, rRNA removal agents may be applied during additional processing operations. The configuration of the constructs generated by such a method can help minimize (or avoid) sequencing of the poly-T sequence during sequencing and/or sequence the 5′ end of a polynucleotide sequence. The amplification products, for example, first amplification products and/or second amplification products, may be subject to sequencing for sequence analysis. In some cases, amplification may be performed using the Partial Hairpin Amplification for Sequencing (PHASE) method.

A variety of applications include the evaluation of the presence and quantification of different biological particle or organism types within a population of biological particles, including, for example, microbiome analysis and characterization, environmental testing, food safety testing, epidemiological analysis, e.g., in tracing contamination or the like.

### Computer Systems

The present disclosure provides computer systems that are programmed to implement methods of the disclosure. FIG. 7 shows a computer system 7 that is programmed or otherwise configured to implement one or more of the methods described herein. For example, computer system 701 may be programmed or otherwise configured to control a microfluidics system (e.g., fluid flow), (ii) sort occupied droplets from unoccupied droplets, (iii) polymerize droplets, (iv) perform sequencing applications, and/or (v) generate and maintain sequencing libraries. The computer system 701 can regulate various aspects of the present disclosure, such as, for example, regulating fluid flow rate in one or more channels in a microfluidic structure, regulating polymerization application units, etc. The computer system 701 can be an electronic device of a user or a computer system that is remotely located with respect to the electronic device. The electronic device can be a mobile electronic device.

The computer system 701 includes a central processing unit (CPU, also “processor” and “computer processor” herein) 705, which can be a single core or multi core processor, or a plurality of processors for parallel processing. The computer system 701 also includes memory or memory location 710 (e.g., random-access memory, read-only memory, flash memory), electronic storage unit 715 (e.g., hard disk), communication interface 720 (e.g., network adapter) for communicating with one or more other systems, and peripheral devices 725, such as cache, other memory, data storage and/or electronic display adapters. The memory 710, storage unit 715, interface 720 and peripheral devices 725 are in communication with the CPU 705 through a communication bus (solid lines), such as a motherboard. The storage unit 715 can be a data storage unit (or data repository) for storing data. The computer system 701 can be operatively coupled to a computer network (“network”) 730 with the aid of the communication interface 720. The network 730 can be the Internet, an internet and/or extranet, or an intranet and/or extranet that is in communication with the Internet. The network 730 in some cases is a telecommunication and/or data network. The network 730 can include one or more computer servers, which can enable distributed computing, such as cloud computing. The network 730, in some cases with the aid of the computer system 701, can implement a peer-to-peer network, which may enable devices coupled to the computer system 701 to behave as a client or a server.

The CPU 705 can execute a sequence of machine-readable instructions, which can be embodied in a program or software. The instructions may be stored in a memory location, such as the memory 710. The instructions can be directed to the CPU 705, which can subsequently program or otherwise configure the CPU 705 to implement methods of the present disclosure. Examples of operations performed by the CPU 705 can include fetch, decode, execute, and writeback.

The CPU 705 can be part of a circuit, such as an integrated circuit. One or more other components of the system 701 can be included in the circuit. In some cases, the circuit is an application specific integrated circuit (ASIC).

The storage unit 715 can store files, such as drivers, libraries and saved programs. The storage unit 715 can store user data, e.g., user preferences and user programs. The computer system 701 in some cases can include one or more additional data storage units that are external to the computer system 701, such as located on a remote server that is in communication with the computer system 701 through an intranet or the Internet.

The computer system 701 can communicate with one or more remote computer systems through the network 730. For instance, the computer system 701 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 701 via the network 730.

Methods as described herein can be implemented by way of machine (e.g., computer processor) executable code stored on an electronic storage location of the computer system 701, such as, for example, on the memory 710 or electronic storage unit 715. The machine executable or machine readable code can be provided in the form of software. During use, the code can be executed by the processor 705. In some cases, the code can be retrieved from the storage unit 715 and stored on the memory 710 for ready access by the processor 705. In some situations, the electronic storage unit 715 can be precluded, and machine-executable instructions are stored on memory 710.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 701, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 701 can include or be in communication with an electronic display 735 that comprises a user interface (UI) 740 for providing, for example, results of sequencing analysis, etc. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 705. The algorithm can, for example, perform a nucleic acid sequencing assay and/or analysis, etc.

Devices, systems, compositions and methods of the present disclosure may be used for various applications, such as, for example, processing a single analyte (e.g., RNA, DNA, or protein) or multiple analytes (e.g., DNA and RNA, DNA and protein, RNA and protein, or RNA, DNA and protein) from a single cell. For example, a biological particle (e.g., a cell or cell bead) is partitioned in a partition (e.g., droplet), and multiple analytes from the biological particle are processed for subsequent processing. The multiple analytes may be from the single cell. This may enable, for example, simultaneous proteomic, transcriptomic and genomic analysis of the cell.

### EXAMPLES

### Example 1: Identifying Sample Misassignment

Provided herein are methods to detect and identify sequencing sample misassignment.

Multi-species single cell ATAC experiments were prepared, barcoded, sequenced, and assessed. Briefly, human B-lymphocyte cell line (GM12878) and mouse T-lymphocyte cell line (EL4) were lysed, and nuclei were permeabilized. Human and mouse nuclei were mixed in a 1:1 ratio prior to tagmentation. Partitions (e.g., droplets) were generated each with a single nucleus and a single gel bead dispersed across thousands of droplets. Tagmented DNA was barcoded in the droplets containing gel beads through linear amplification and samples were indexed via PCR. Multiple sequencing libraries were pooled and sequenced in parallel (e.g., on an Illumina NovaSeq flowcell) and analyzed.

The sequencing reads were analyzed by using a barnyard plot which shows all the barcodes and their number of associated fragments from each species. For each barcode, the numbers of sequencing reads/fragments per barcode from mouse or human origin were plotted. If the sequencing reads do not contain substantial sample misassignment, each barcode associated with a cell contain a high number of either human or mouse, but not both, sequence reads/fragments. During fragment amplification on the sequencer, the chemistry on the flowcell may allow a small fraction of molecules to “hop” to a different molecule with a different index. This results in fragments from one sample (e.g. human PBMC sample) to be mapped back to a different sample (e.g. a human:mouse barnyard mix sample). When sample misassignment occurs, a significant portion of barcodes, especially those associated with cells, contain a lower number of reads/fragments. In some cases, a significant portion of cell-associated barcodes can also contain sequence reads/fragments from both species.

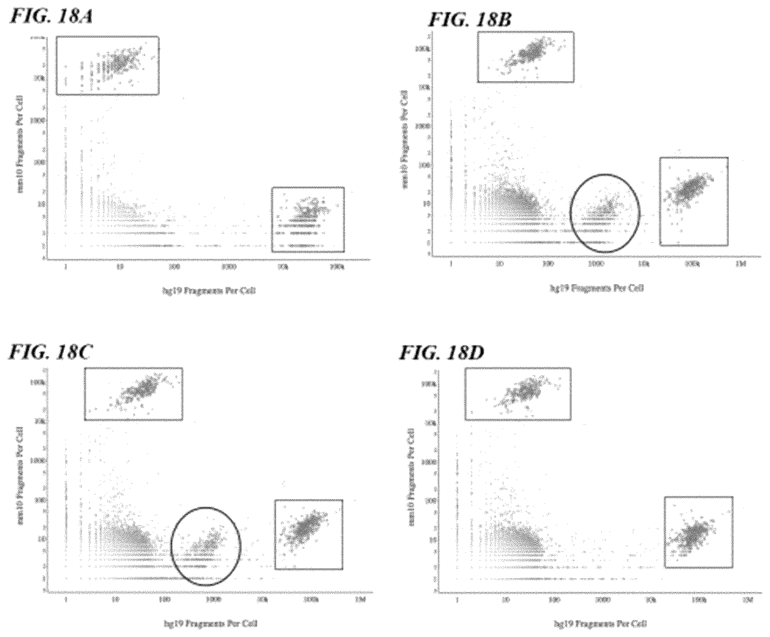

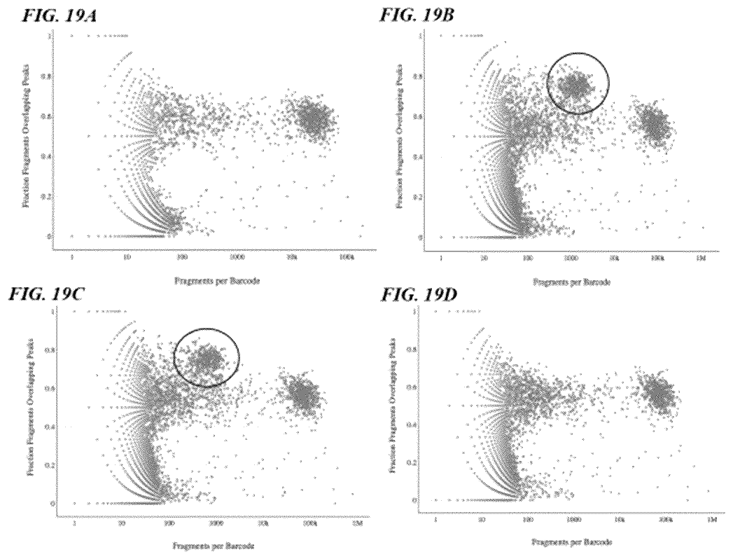

FIG. 18A-D illustrates barnyard plots used to assess sample assignment from the mouse-human multi-species experiments. The four barnyard plots show the number of barcoded mouse sequence reads and human sequence reads on the y- and x-axis, respectively. The barcodes associated with a cell are boxed while the remaining barcodes are those not associated with a cell. In FIG. 18A (45k paired RPC), misassignment of indexes was not detected; two populations of cell-associated barcodes contained a high number of sequence reads, about 10k per barcode, from either the mouse or human sequence alone. In FIG. 18B, a multi-species experiment sequenced 25 times deeper than a normal depth (630k paired RPC) showed that at least some of the samples were not correctly assigned. A significant portion of barcodes, the ones not associated with any cells and highlighted by a circle, contained a low number of sequence reads (˜1,000 reads per barcode) that arose due to index hopping of a human PBMC fragment to an index of a barnyard sample. In FIG. 18C, the sample from FIG. 18B was computationally downsampled (300k paired RPC) to the same depth as in FIG. 18D. However, index hopping was still observed as shown in FIG. 18C, showing that at this depth, index hopping may occur if another sample was sequenced on the same flowcell. In FIG. 18D, the multi-species experiment was sequenced ˜10 times deeper than a normal depth (280k paired RPC), as in the experiment in FIG. 18C. However, each sequencing reaction was run in an independent lane. As shown in FIG. 18D, the samples were correctly assigned, suggesting that the missassignment was due to index hopping between samples on the same flowcell and not solely due to increased read depth. Not only that two populations of cell-associated barcodes contained a high number of sequence reads, about 10k per barcode, from either mouse or human sequence alone, but a minimal number of barcodes contained a low number of sequence reads.

Single-cell targeting plots were also used to assess sample assignment. FIG. 19A-D shows targeting plots analyzing the sequencing reads assessed in FIG. 18A-D. The four single-cell targeting plots show the number of on-target sequence reads/fragments, or sequence reads that overlap peaks, on the y-axis and the number of sequence reads per barcode on the x-axis. Similar to FIG. 18A-D, index hopping was observed in some samples as described above. For example, index hopping/sample misassignment was observed in samples shown in FIG. 19B and FIG. 19C (630k paired RPC and 300k paired RPC, respectively) indicated by the circled population. In comparison, index hopping was not observed in samples shown in FIG. 19A (45k paired RPC) and FIG. 19D (280k paired RPC, split lanes).

### Example 2: Example Workflow for Dual Indexing

Provided herein are example methods for preparing samples, generating barcoded tagmented DNA fragments, and sequencing the generated barcoded molecules.

Samples containing cells are lysed and the nuclei are permeabilized. For tagmentation, the nucleic acid molecules (e.g., DNA molecule) are tagmented using transposase-nucleic acid complexes comprising a transposase and a transposon end sequence and a sequencing primer/tag sequence (e.g., R1 or R2). Partitions (e.g., droplets) are then generated each with a single nucleus and a single gel bead dispersed across thousands of droplets. Tagmented DNA is barcoded in the droplets containing gel beads through linear amplification and samples are indexed via PCR (e.g., as shown in FIG. 12). For example, the tagmented nucleic acid (e.g., DNA fragment) undergoes barcoding with beads within a partition such that tagmented fragments from a single nucleus are labeled with the same barcode. The nucleic acid barcode molecule attached to the gel bead contains: a spacer sequence (e.g., first primer binding sequence)-a barcode sequence-a tag binding sequence (e.g., sequencing primer R1 or R2) or portion thereof or complement thereof. In some cases, the tag binding sequence of the nucleic acid barcode molecule hybridizes to the tag sequence of the tagmented nucleic acid (e.g., DNA fragment) and the amplification reaction is performed.

The barcoded fragments are released from the partition and processed for dual indexing with a pair of distinct first and second sample identification sequences (e.g., i5 and i7 indexes) using SI-PCR primers (e.g., as shown in FIG. 12). The first SI-PCR comprises: 5′-a first flow cell adaptor sequence or complementary sequence thereof (e.g., P5)-a first sample identification sequence or complementary sequence thereof (e.g., i5)-a first primer sequence (a spacer or complementary sequence thereof)-3′ and the second SI primer comprises: 5′-a second flow cell adaptor sequence (e.g., P7)-a second sample identification sequence (e.g., i7)-a second sequencing primer/tag binding sequence (e.g., R2′)-3′, e.g., as shown in FIG. 17. The generated nucleic acid molecule that is barcoded and dual indexed may comprise: a first flow cell adaptor sequence (e.g., a P5 sequence)-a first sample identification sequence (e.g., an i5 index sequence)-a first primer sequence (e.g., a spacer sequence)-a barcode sequence-a first sequencing primer/tag sequence (e.g., R1 or R2 sequence)-a tagmented fragment-a second sequencing primer/tag sequence (e.g., R1 or R2 sequence)-a second sample identification sequence (e.g., an i7 index sequence)-a second flow cell adaptor sequence (e.g., a P7 sequence), or a complement of any of the sequences thereof, e.g., as shown in FIG. 14.

Multiple sequencing libraries are pooled and sequenced in parallel and analyzed. For sequencing, four reads are generated: a first read comprising: a first sample identification sequence or complementary sequence thereof (e.g., i5)-a first primer or primer binding sequence (a spacer or complementary sequence thereof)-a barcode sequence or complement thereof; a second read comprising a second sample identification sequence or complementary sequence thereof (e.g., i7); a third read comprising the tagmented DNA fragment (e.g., ATAC fragment) or portion thereof; and a fourth read comprising the tagmented DNA fragment (e.g., ATAC fragment) or portion thereof. The tagmented DNA fragment (e.g., ATAC fragment) reads can be generated using sequencing primers complementary to the flanking sequencing primer/tag sequence or complements or portions thereof (e.g., R1 and/or R2) to sequence both ends of the fragment. Sequences with unexpected index pairs can be identified, filtered out or removed from the analysis.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method for detecting nucleic acid index hopping, comprising:
(a) appending index sequences to a plurality of nucleic acid molecules, wherein: (i) a first nucleic acid molecule of the plurality of nucleic acid molecules comprises a first index sequence and a second index sequence; and (ii) the first index sequence and the second index sequence are different index sequences;
(b) obtaining sequence reads from the first nucleic acid molecule, wherein sequencing reads obtained from the first nucleic acid molecule comprise an index sequence different from the first index sequence and the second index sequence; and
(c) determining that the first nucleic acid molecule, prior to or during (b), was subject to index hopping from the sequencing reads obtained from the first nucleic acid molecule.

2. The method of claim 1, wherein, in (a), a second nucleic acid molecule of the plurality of nucleic acid molecules comprises a third index sequence and a fourth index sequence that are different from each other and each different from the first index sequence and the second index sequence.

3. The method of claim 2, further comprising obtaining sequence reads from the second nucleic acid molecule, wherein sequence reads obtained from the second nucleic acid molecule comprise one of the first index sequence and the second index sequence.

4. The method of claim 3, further comprising determining that the second nucleic acid molecule, prior to or during (b), was subject to index hopping from the sequencing reads obtained from the second nucleic acid molecule.

5. The method of claim 1, wherein, in (a), a second nucleic acid molecule of the plurality of nucleic acid molecules comprises the first index sequence and the second index sequence.

6. The method of claim 5, further comprising: (i) obtaining sequencing reads from the second nucleic acid molecule; (ii) further processing the sequencing reads obtained from the second nucleic acid molecule; and (iii) withholding the sequencing reads from the first nucleic acid molecule from further processing.

7. The method of claim 5, wherein the first nucleic acid molecule or the second nucleic acid molecule comprises a barcode sequence, a unique molecular identifier, or both.

8. The method of claim 1, further comprising generating the first nucleic acid molecule by contacting a molecule comprising a tagged deoxyribonucleic acid (DNA) fragment sequence from a cell or cell nucleus of a sample of a plurality of samples, or complement thereof, and a first tag sequence separate from the index sequences, with a nucleic acid barcode molecule comprising: (i) a first primer binding sequence, (ii) a barcode sequence, and (iii) a first tag binding sequence complementary to the first tag sequence, to generate a first barcoded molecule.

9. The method of claim 8, further comprising coupling the first index sequence and the second index sequence to the first barcoded molecule, or derivative thereof, to generate the first nucleic acid molecule.

10. The method of claim 9, wherein the coupling comprises a nucleic acid extension reaction.

11. The method of claim 8, wherein the nucleic acid barcode molecule is coupled to a support.

12. The method of claim 11, wherein the nucleic acid barcode molecule comprises, from 5′ to 3′: (i) the first primer binding sequence, (ii) the barcode sequence, and (iii) the first tag binding sequence, and wherein a 5′ end of the nucleic acid barcode molecule is coupled to the support.

13. The method of claim 11, wherein the support is a bead.

14. The method of claim 11, wherein the support is coupled to a plurality of nucleic acid barcode molecules comprising the nucleic acid barcode molecule.

15. The method of claim 11, wherein the nucleic acid barcode molecule is releasably coupled to the support via a labile moiety.

16. The method of claim 8, wherein the molecule comprising the tagged DNA fragment is flanked at one end with the first tag sequence and flanked at its other end with a second tag sequence.

17. The method of claim 16, wherein appending the index sequences comprises providing a first primer comprising (i) the first index sequence and (ii) a priming sequence complementary to the first primer binding sequence.

18. The method of claim 17, wherein appending the index sequences comprises providing a second primer comprising (i) a second tag binding sequence complementary to the second tag sequence, (ii) the second index sequence, and (iii) a second adapter sequence.

19. The method of claim 1, further comprising partitioning the plurality of nucleic acid molecules into a plurality of partitions.

20. The method of claim 1, further comprising deep sequencing the first nucleic acid molecule.
